(19) United States
(12) Patent Application Publication
(10) Pub. No.: US 2018/0362590 A1
(43) Pub. Date: Dec. 20, 2018

(54) POLYPEPTIDES WITH TYPE V CRISPR ACTIVITY AND USES THEREOF

(71) Applicant: Synthetic Genomics, Inc., La Jolla, CA (US)

(72) Inventors: Russell David Monds, San Diego, CA (US); Simone Moraes Mantovani, San Diego, CA (US); Russell S. Komor, San Diego, CA (US); Matthew Carey LaFave, Escondido, CA (US); Krishna Kannan, San Diego, CA (US); Joseph W. LaMattina, San Diego, CA (US); Joseph S. Lucas, San Diego, CA (US); Eric R. Moellering, San Diego, CA (US)

(21) Appl. No.: 15/953,286

(22) Filed: Apr. 13, 2018

Related U.S. Application Data
(60) Provisional application No. 62/657,489, filed on Apr. 13, 2018.
(60) Provisional application No. 62/586,852, filed on Nov. 15, 2017.
(60) Provisional application No. 62/485,796, filed on Apr. 14, 2017.

(51) Int. Cl. C07K 14/00; C12N 15/113; C12P 19/34; C12N 15/11; C12N 15/62; C12N 9/22

(52) U.S. Cl. CPC C07K 14/00; C12N 15/113; C12P 19/34; C12N 15/111; C07K 2319/10; C12N 9/22; C12N 2310/20; C07K 2319/03; C12N 15/62

(57) ABSTRACT

Disclosed herein are novel polypeptides having nuclease activity. The Mmc3 polypeptides function as Class 2 Type V effectors, and catalyze double stranded breaks in nucleic acid strands. The polypeptides are useful, for example, for gene editing systems such as CRISPR, to make site specific alterations of target nucleic acid sequences.

### CROSS-REFERENCE TO RELATED APPLICATIONS

This application claims benefit of priority under 35 U.S.C. § 119(e) of U.S. Ser. No. 62/485,796, filed Apr. 14, 2017, to U.S. Ser. No. 62/586,852, filed Nov. 15, 2017, and to U.S. Ser. No. 62/657,489, filed Apr. 13, 2018, the entire contents of which are incorporated herein by reference in their entireties.

### INCORPORATION BY REFERENCE OF SEQUENCE LISTING

The material in the accompanying sequence listing is hereby incorporated by reference into the application. The accompanying sequence listing text file, name SGI2090_2_Sequence_Listing, was created on Apr. 12, 2018, and in 642 kb. The file can be accessed using Microsoft Word on a computer that uses Window OS.

### FIELD OF THE INVENTION

The present invention relates generally to polypeptides which effect breaks at defined locations within DNA and more specifically the use of such polypeptides for gene editing.

### BACKGROUND OF THE INVENTION

Certain prokaryotes (some bacteria and most archaea) display primitive adaptive immunity against bacteriophage infections, and can eliminate the invading genetic material. The CRISPR/Cas system is an example of such a prokaryotic immune system. Clustered regularly interspaced short palindromic repeats (CRISPR) are segments of prokaryotic DNA containing short, repetitive base sequences (for example, up to 100 identical repeats of 25-40 base pairs). Each CRISPR repeat sequence is followed by short segments of interspersed exogenous “spacer” DNA from previous “infections”, i.e., exposure to viruses, phage, or plasmids. CRISPR clusters are transcribed as multi-unit precursors that are subsequently cleaved into smaller units, and processed to form guide CRISPR RNAs (guide RNA) that consist of one spacer flanked by sequence derived from a CRISPR repeat. CRISPR loci also contain one or more genes encoding Cas proteins. The guide RNA harboring the spacer sequence directs Cas proteins to exogenous invading DNA and allows the enzyme to cleave it, thereby conferring a type of resistance against the invader. DNA is recognized for cleavage not only by its homology to a spacer sequence of the CRISPR cluster, but also by its proximity to a protospacer adjacent motif (PAM), a sequence that is typically 2-6 nucleotides in length.

The CRISPR/Cas system has been adapted to manipulate DNA in situ, permitting gene editing within cells. The CRISPR/Cas9 system is an example of the original application, and provides a system that delivers a Cas9 nuclease complexed with a synthetic guide RNA (gRNA) into a target cell. The cell's genome is cut at a specified location. This permits further modification of the target cell genome. However, the CRISPR system is limited by the PAM sequence requirements of the Cas9 and Cpf1 nucleases (reviewed in Hu et al. (2014) Cell, 157: 1262-1278 and Zetsche et al. (2015) Cell 163: 759-771). CRISPR systems have also demonstrated differential ability to target different sites in the genome. Part of this is due to PAM restrictions, however, some of these differences are seemingly due to less defined characteristics of the different systems. There remains a need in the art for unique nucleases that can be used in engineered CRISPR systems that can be used to edit nucleic acids in a variety of contexts, that can expand the potential of these systems for directed gene editing.

### SUMMARY OF THE INVENTION

Described herein are gene editing (CRISPR) systems, that are useful for modifying target nucleic acid sequences. Accordingly, provided herein is an engineered or non-naturally-occurring CRISPR-Cas system that includes an engineered guide RNA comprising a guide sequence, where the guide sequence is capable of hybridizing with a target sequence of a target nucleic acid molecule, and an Mmc3 effector or a nucleic acid molecule encoding an Mmc3 effector, where the engineered guide RNA and the Mmc3 effector protein do not naturally occur together. The guide RNA can comprise at least a portion of a CRISPR repeat of an Mmc3 Type V CRISPR Cas system. The target sequence, the guide RNA, and the effector form a complex which causes cleavage of the target molecule distal to a protospacer adjacent motif (PAM), where the target sequence of the target molecule can be 3′ of the PAM.

Also provided herein is an engineered or non-naturally-occurring CRISPR-Cas system having a polynucleotide sequence that encodes an engineered guide RNA and an Mmc3 effector or a nucleic acid molecule encoding an Mmc3 effector, where the engineered guide RNA and the Mmc3 effector protein do not naturally occur together. The polynucleotide sequence that encodes an engineered guide RNA and the polynucleotide sequence that encodes an Mmc3 effector can be operably linked to regulatory elements. A regulatory element operably linked to a nucleic acid sequence encoding an Mmc3 effector and/or a regulatory element operably linked to a nucleic acid sequence encoding a guide RNA can be a promoter, such as a promoter that is active in a host cell of interest. A regulatory element operably linked to either or both of an effector gene or a guide RNA gene can be inducible. The expression cassettes for the guide RNA and the Mmc3 effector can be on the same or different nucleic acid molecules. The guide sequence of the guide RNA is capable of hybridizing with a target sequence of a target nucleic acid molecule and can hybridize with a target sequence 3′ of a protospacer adjacent motif (PAM). The target sequence, the guide RNA, and the effector form a complex which causes cleavage of the target molecule distal to the PAM.

In various embodiments of engineered or non-naturally occurring CRISPR-Cas systems provided herein the Mmc3 effector polypeptide can comprise:

an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or

a variant of an Mmc3 effector comprising an amino acid sequence having at least 60%, 65%, 70%, 75%, 80%, 85%, 90%, or 95% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or

a naturally-occurring Mmc3 effector comprising an amino acid sequence having at least 50%, 55%, 60%, 65%, 70%, 75%, 80%, 85%, 90%, or 95% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26.

An engineered or non-naturally occurring CRISPR system that includes an Mmc3 effector polypeptide or a gene encoding an Mmc3 effector polypeptide as provided herein can be used to modify a target nucleic acid molecule in vitro or in vivo and can be used for in vivo editing of nucleic acid molecules in prokaryotic or eukaryotic cells. The target nucleic acid molecule can be a DNA molecule, and can be an episomal DNA molecule within a cell of interest, or can be genomic (e.g., chromosomal) DNA. The target DNA can also be an isolated nucleic acid molecule, for example, where the system is used for in vitro target modification.

Mmc3 effector polypeptides comprise a family of Class 2, Type V traguide RNA-independent RNA-guided nucleases, or effector polypeptides, as disclosed in detail herein. The Mmc3 family forms a distinct group of RNA-guided endonucleases that include an RuvC domain characterized by three catalytic motifs with characteristic spacing. The Mmc3 effectors lack a nuc domain and include a zinc finger domain characterized by two cysteine pairs that occur between the second and third RuvC motifs. An Mmc3 effector used in the systems and methods provided herein can be any Mmc3 effector, including any disclosed herein, orthologs thereof, and variants thereof. In various embodiments, the effector is derived from a bacterial species, such as but not limited to a species of the order Bacteriodales, or any of the genera Bacteroides, Porphyromonas, Sulfuricurvum, Smithella, Candidatus, or Omnitrophica. In some embodiments the effector includes a nuclear localization signal and/or the gene encoding the effector is codon optimized for expression or function in a host cell of interest, which can be, for example, a eukaryotic cell. An Mmc3 effector as provided herein can include, for example, a nuclear localization sequence (NLS) and/or a purification tag or detection tag or can include a labeling moiety directly or indirectly conjugated or bound to the protein.

The guide RNA, or crRNA, in various embodiments does not include tracr sequences and can include at least a portion of one or more repeat sequences of a naturally-occurring Mmc3 CRISPR system. The CRISPR repeat sequences of the guide RNA can be 5′ of the guide sequence, and can be positioned both 5′ and 3′ of the guide (target) sequence. A guide RNA can be produced within the target cell, for example, by transcription within the cell of a CRISPR array (CRarray) or portion thereof or of a construct that includes a guide sequence (sometimes referred to as the spacer sequence or target sequence) juxtaposed with one or more sequences derived from a CRISPR repeat, or can be synthesized in vitro, for example, by in vitro transcription of a DNA construct or by chemical synthesis. A guide RNA used in the systems disclosed herein can optionally include modifications, such as but not limited to phosphorothioates or 2′-OMe groups. A guide RNA can optionally include one or more deoxynucleotides.

The guide RNA forms a complex with an Mmc3 effector protein and causes cleavage of one or more target nucleic acid molecules having a sequence homologous to the guide sequence (also called the targeting sequence or spacer sequence) of the guide RNA. In various examples, the percentage of nucleic acid molecule cleavage performed by an Mmc3 system as provided herein can be from about 4% to 100%. Systems and methods provided herein can include two or more guide RNAs or nucleotide sequences encoding guide RNAs that have different guide sequences. The guide RNAs in some examples can target the different sites on the same target nucleic acid molecule which can optionally be different sites within the same gene. Alternatively, the two or more guide RNAs can target different genes or different target nucleic acid molecules.

In some embodiments of the nucleic acid editing systems provided herein, a guide RNA complexed with an Mmc3 effector protein is provided. The complexed guide RNA and Mmc3 effector can be used for in vitro DNA modification, or can be delivered to a cell, for example, by electroportation, peptide-mediated protein delivery, liposome delivery, biolistics, or other methods, for in vivo modification of or binding to target DNA.

In some embodiments of the nucleic acid editing systems provided herein, the CRISPR-Cas system further includes an Mmc3 ORF3 polypeptide or a polynucleotide sequence encoding an Mmc3 ORF3 polypeptide. The ORF3 polypeptide can be any ORF3 polypeptide of an Mmc3 system or a variant thereof having at least 60%, at least 65%, at least 70%, at least 75%, at least 80%, at least 85%, at least 90%, at least 95%, at least 98%, or at least 99% to a naturally-occurring Mmc3 ORF3 polypeptide. Exemplary Mmc3 ORF3 polypeptide include those comprising amino acid sequence of SEQ ID NO:50-58. In some embodiments a CRISPR-Cas system includes a polynucleotide sequence encoding a guide RNA, a nucleic acid molecule encoding an effector polypeptide, such as an Mmc3 or Cpf1 effector polypeptide, and a polynucleotide sequence encoding an ORF3 polypeptide. The guide RNA construct and the nucleic acid molecules encoding the effector and ORF3 polypeptides can be operably linked to regulatory elements. One of more of the regulatory elements can be an inducible promoter.

Further included herein is a method of modifying one or more target nucleic acid molecules in vivo, where the method comprises delivering to a cell that includes at least one target molecule comprising at least one target sequence a) one or more guide RNAs, or one or more nucleotide sequences encoding one or more guide RNAs, and b) an Mmc3 effector polypeptide, or a gene encoding an Mmc3 effector polypeptide, where the guide RNA is engineered to target a nucleic acid molecule in the cell. In various examples, the guide RNA and Mmc3 effector do not naturally occur together and/or do not naturally occur in the cell to which they are delivered. The guide RNA has homology to a sequence in the target nucleic acid molecule, and the target nucleic acid molecule can be modified by the Mmc3 effector complexed with the guide RNA. The rate of target nucleic acid modification can be at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 99%. The target nucleic acid molecule can be a DNA molecule. The modification can be cleavage, or can be mutation, for example, by nucleotide changes, delection of nucleotides, or insertion of one or more nucleotide that may occur during repair processes following cleavage by the Mmc3 effector. In some embodiments, the method can further include delivering a donor or repair template sequence to the cell. The donor or repair sequence can optionally include sequences that mediate homologous recombination into the targeted locus, e.g., sequences having a homology to sequences at or proximal to the target site (protospacer) of the nucleic acid molecule. In some embodiments, the donor or repair fragment can include a selectable marker.

The methods of modifying one or more target nucleic acid molecules in vivo can further include delivering to the target cell an Mmc3 ORF3 polypeptide or a polynucleotide sequence encoding an Mmc3 ORF3 polypeptide. Also included are methods of modifying one or more target nucleic acid molecules in vivo, where the method comprises delivering to a cell that includes at least one target molecule comprising at least one target sequence a) one or more guide RNAs, or one or more nucleotide sequences encoding one or more guide RNAs, b) an Mmc3 effector polypeptide, or a gene encoding an Mmc3 effector polypeptide, and c) an Mmc3 ORF3 polypeptide, where the guide RNA is engineered to target a nucleic acid molecule in the cell. In various examples, the guide RNA and Mmc3 effector do not naturally occur together and/or do not naturally occur in the cell to which they are delivered. The guide RNA has homology to a sequence in the target nucleic acid molecule, and the target nucleic acid molecule can be modified by the Mmc3 effector complexed with the guide RNA. The rate of target nucleic acid modification can be greater than the rate of target nucleic acid modification performed by the same CRISPR-cas system that lacks an Mmc3 ORF3 polypeptide. The effector polypeptide can by an Mmc3 or Cpf1 effector polypeptide. The target nucleic acid molecule can be a DNA molecule. The modification can be cleavage, or can be mutation, for example, by nucleotide changes, delection of nucleotides, or insertion of one or more nucleotide that may occur during repair processes following cleavage by the effector. In some embodiments, the method can further include delivering a donor or repair template sequence to the cell. The donor or repair sequence can optionally include sequences that mediate homologous recombination into the targeted locus, e.g., sequences having a homology to sequences at or proximal to the target site (protospacer) of the nucleic acid molecule. In some embodiments, the donor or repair fragment can include a selectable marker.

The methods provided herein can use any Mmc3 effector polypeptide, including but not limited to any disclosed herein, e.g., any of the Mmc3 effectors listed in Table 2 or Table 3, or variants thereof having at least 60%, 65%, 70%, 75%, 80%, 85%, 90%, or 95% identity thereto.

The host cell can be a prokaryotic cell or a eukaryotic cell. For example, the method can be a method of modifying one or more target nucleic acid molecules in a eukaryotic cell, such as an animal or plant cell, or a fungal, algal, or labyrinthulomycete cell. The nucleotide sequence encoding the Mmc3 effector protein can be codon optimized for expression in a target cell, such as a eukaryotic cell, and/or can include one or more nuclear localization signal(s) (NLS(s)). In some embodiments of the methods, a sequence encoding a guide RNA and a sequence encoding an Mmc3 effector protein are provided on the same vector. The guide RNA-encoding sequence and Mmc3-encoding squence can be operably linked to regulatory elements, such as promoters. In other embodiments, the guide RNA and Mmc3 effector polypeptide are provided to the cell as a complex. In further embodiments, a host cell of interest that is transformed with a gene encoding an Mmc3 effector protein is subsequently transformed with a guide RNA targeting a host target nucleic acid molecule. In some examples, a host cell of interest expresses a transgene encoding an Mmc3 effector protein prior to being transformed with a guide RNA targeting the host target nucleic acid molecule. The guide RNA introduced into the host cell can optionally be chemically modified, such as with phosphorothioate or one or more 2′-OMe groups and/or can include one or more deoxynucleotides.

In some embodiments, the method is a method of modifying one or more target nucleic acid molecules in a eukaryotic cell, where the method comprises delivering to a eukaryotic cell that includes at least one target molecule comprising at least one target sequence a) one or more guide RNAs, or one or more nucleotide sequences encoding one or more guide RNAs, and b) an Mmc3 effector polypeptide, or a gene encoding an Mmc3 effector polypeptide, where the guide RNA is engineered to target a nucleic acid molecule in the cell, where the target nucleic acid molecule sequence is modified by the Mmc3 effector and the target DNA sequence modification rate is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 99%. The Mmc3 effector can comprise, for example:

an amino acid sequence selected from the group consisting of SEQ ID NOs:1-26; or

a variant of an Mmc3 effector comprising an amino acid sequence having at least 60%, 65%, 70%, 75%, 80%, 85%, 90%, or 95% identity to any of SEQ ID NOs:1-25; or

a naturally-occurring Mmc3 effector having at least 50% identity to any of SEQ ID NOs:1-25.

For example, the method of modifying a nucleic acid molecule in a eukaryotic cell, such as a fungal, algal, plant, or animal cell, can include delivering to a eukaryotic cell that includes at least one target molecule comprising at least one target sequence a) one or more guide RNAs, or one or more nucleotide sequences encoding one or more guide RNAs, and b) an Mmc3 effector polypeptide, or a gene encoding an Mmc3 effector polypeptide, where the Mmc3 effector polypeptide comprises an amino acid sequence having at least 50%, 55%, 60%, 65%, 70%, 75%, 80%, 85%, 90%, or 95% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26. The guide RNA is engineered to target a nucleic acid molecule in the cell, and the target nucleic acid molecule sequence is modified by the Mmc3 effector where the target DNA sequence modification rate is at least 5%, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 99%. In some embodiments the Mmc3 effector polypeptide has at least 50%, 55%, 60%, 65%, 70%, 75%, 80%, 85%, 90%, or 95% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:16, SEQ ID NO:17, SEQ ID NO:20, or SEQ ID NO:21. In some embodiments the Mmc3 effector polypeptide has at least 50%, 55%, 60%, 65%, 70%, 75%, 80%, 85%, 90%, or 95% identity to SEQ ID NO:1, SEQ ID NO:2, SEQ ID NO:3, SEQ ID NO:5, SEQ ID NO:6, or SEQ ID NO:7.

Also provided herein is a cell or organism engineered to express an Mmc3 effector polypeptide, where the Mmc3 effector polypeptide is not native to the engineered cell or organism. In various embodiments the Mmc3 effector polypeptide can comprise:

an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or

a variant of an Mmc3 effector comprising an amino acid sequence having at least 60%, 65%, 70%, 75%, 80%, 85%, 90%, or 95% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26; or

a naturally-occurring Mmc3 effector having at least 50% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26.

The cell or organism can be a prokaryotic or eukaryotic cell or organism that does not naturally include a gene encoding an Mmc3 effector, and can be, for example, a fungal, labyrinthulomycete, algal, plant, animal, avian, reptile, amphibian, fish, cephalopod, crustacean, insect, arachnid, marsupial, or mammalian cell or organism. The gene encoding an Mmc3 effector that in non-native with respect to the host organism can be operably linked to a regulatory element, such as a promoter. The promoter can be native to the host organism or can be a promoter of another species. A construct for expressing an Mmc3 effector in a heterologous host, such as a eukaryotic organism, can optionally further include a terminator. The gene encoding the Mmc3 effector can optionally be codon optimized for the host species, can optionally include one or more introns, and can optionally include one or more peptide tag sequences, one or more nuclear localiztion sequences (NLSs) and/or one or more linkers or engineered cleavage sites (e.g., a 2a sequence). In various embodiments a cell or organism can include any of the engineered Mmc3 CRISPR systems disclosed above, where the nucleic acid sequence encoding the effector is present in the cell prior to introduction of a guide RNA. In other embodiments, the cell that in engineered to include a gene for expressing an Mmc3 effector polypeptide can further include a polynucleotide encoding a guide RNA (e.g., a guide RNA) that is operably linked to a regulatory element. In some embodiments the Mmc3 effector polypeptide has at least 50%, 55%, 60%, 65%, 70%, 75%, 80%, 85%, 90%, or 95% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:16, SEQ ID NO:17, SEQ ID NO:20, or SEQ ID NO:21. In some embodiments the Mmc3 effector polypeptide has at least 50%, 55%, 60%, 65%, 70%, 75%, 80%, 85%, 90%, or 95% identity to SEQ ID NO:1, SEQ ID NO:2, SEQ ID NO:3, SEQ ID NO:5, SEQ ID NO:6, or SEQ ID NO:7.

Also provided herein are Mmc3 effector polypeptides and genes encoding such Mmc3 effector polypeptides. In one aspect, Mmc3 effector polypeptides comprising amino acid sequences selected from the group consisting of SEQ ID NOs: 1, 2, 3, 4, 5, 6, 7, 8, 9, 17, 18, 19, 20, 21, 22, 23, 24, 25, and 26 or variants having at least least 50%, at least 55%, at least 60%, at least 65%, at least 70%, at least 75%, at least 80%, at least 85%, at least 90%, at least 95%, at least 98%, or at least 99% thereto, are provided, where the effector polypeptides are outside of the prokaryotic species they are native to. For example, the Mmc3 effector polypeptides may be partially or substantially purified away from other cellular components and may be, as nonlimiting examples, outside the context of a cell, in solution (liquid or frozen) or in particulate (solid) form (for example, in a precipitate, in a crystalline form, and/or as a lyophilate), or may be in a cell that the Mmc3 effector is not naturally found in, which may be a prokalryotic or eukaryotic cell. The polypeptides can include one or more non-Mmc3 amino acid sequences, such as but not limited to, an NLS or a purification or detection tag. In some embodiments, the polypeptides can have at least one mutation the results in reduced nuclease activity, as disclosed herein. The polypeptides can by part of fusion proteins, such as, for example, with a fluorescent protein, a DNA modifying enzyme, or a transcriptional activation domain

Also provided are nucleic acid molecules encoding Mmc3 effector polypeptides comprising amino acid sequences selected from the group consisting of SEQ ID NOs: 1, 2, 3, 4, 5, 6, 7, 8, 9, 17, 18, 19, 20, 21, 22, 23, 24, 25, and 26, or variants having at least 50%, at least 55%, at least 60%, at least 65%, at least 70%, at least 75%, at least 80%, at least 85%, at least 90%, at least 95%, at least 98%, or at least 99% identity thereto. The Mmc3 genes can be codon-optimized for a species in which expression of the Mmc3 effector is desired, and can optionally include one or more introns that can optionally be derived from the species in which the Mmc3 gene is to be expressed. An Mmc3 gene as provided herein can encode an Mmc3 polypeptide that includes an NLS and/or a purification or detection tag. An Mmc3 gene as provided herein can encode a fusion protein that includes the Mmc3 polypeptide translationally linked to another polypeptide such as, for example, a fluorescent protein. A nucleic acid molecule that includes a nucleic acid sequence encoding an Mmc3 effector polypeptide can be an expression cassette that includes a promoter operably linked to the nucleic acid sequence encoding the Mmc3 effector polypeptide. A nucleic acid molecule encoding an Mmc3 effector polypeptide can be a vector that includes one or both of an origin of replication and a selectable marker.

Further provided is a nuclease-deficient mutant of an Mmc3 effector polypeptide, such as any disclosed herein, including an Mmc3 polypeptide having at least at least 50%, at least 55%, at least 60%, at least 65%, at least 70%, at least 75%, at least 80%, at least 85%, at least 90%, at least 95%, at least 98%, or at least 99% identity to any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26. The nuclease-deficient mutant polypeptide can be mutated in at least one amino acid of the RuvCI motif or at least one amino acid of the RuvCII motif, for example, may have a mutation of the amino acid corresponding to D841 or E1061 of BdMmc3 (SEQ ID NO:1). In some embodiments the mutation is a mutation of aspartate or glutamate to alanine.

In another aspect provided herein is a CRISPR system that comprises: at least one effector polypeptide or a nucleic acid construct for expressing an effector polypeptide, at least one guide RNA or a nucleic acid construct for expressing a guide RNA, and at least one Mmc3 ORF3 polypeptide or a nucleic acid construct for expressing an Mmc3 ORF3 polypeptide. The CRISPR system effector polypeptide can be, for example, an Mmc3 effector or a variant thereof, such as any disclosed herein, or a Cpf1 effector or a variant thereof, including Cpf1 effectors known in the art and disclosed herein. An ORF3 polypeptide can be any Mmc3 ORF3 polypeptide (e.g., any of SEQ ID NOs:50-58, or a variant having at least 50%, at least 55%, at least 60%, at least 65%, at least 70%, at least 75%, at least 80%, at least 85%, at least 90%, at least 95%, at least 98%, or at least 99% identity thereto. Provided herein is a CRISPR system comprising a Cpf1 effector having at least 95% identity to SEQ ID NO:200 (Smp2Cpf1). The CRISPR system includes a Cpf1 effector having at least 95% identity to SEQ ID NO:200 or a polynucleotide sequence encoding a Cpf1 effector having at least 95% identity to SEQ ID NO:200 and a guide RNA, where the Cpf1 effector and guide RNA do not naturally occur together, and optionally further includes an Mmc3 ORF3 polypeptide, or a polynucleotide sequence encoding an Mmc3 ORF3 polypeptide.

A CRISPR system as disclosed herein can include an Mmc3 ORF3 polypeptide that can, for example, introduced into cells by electroporation, biolistics, peptide protein transporters, liposomes, or other protein delivery vehicles. An Mmc3 ORF3 polypeptide can be delivered to a target cell along with an effector polypeptide, or, for example, an ORF3 polypeptide can be introduced into a target cell that includes an expression construct for producing an effector polypeptide, and optionally, a guide RNA. Alternatively, an Mmc3 ORF3 polypeptide can be delivered to a target cell along with a guide RNA, or can be delivered to a cell that includes a guide RNA expression construct or will independently be transfected with a guide RNA. A CRISPR system can alternatively include a polynucleotide sequence encoding an Mmc3 ORF3 polypeptide. A gene encoding an Mmc3 ORF3 polypeptide can be codon-optimized for expression in the target cell, and can optionally include a sequence encoding an NLS and/or a peptide tag.

Further provided is a cell engineered to express an Mmc3 ORF3 polypeptide. As disclosed herein, a host cell engineered to express an ORF3 cell is a cell that does not naturally include an ORF3 gene, and may be, for example, a eukaryotic cell. The ORF3 gene can be codon-optimized and can encode one or more NLSs in the ORF3 coding sequence, for example at the N or C terminus of the ORF3 polypeptide.

Further provided is a method for genome modification, comprising delivering to a cell that includes at least one target molecule comprising at least one target sequence a) one or more guide RNAs, or one or more nucleotide sequences encoding one or more guide RNAs, b) an effector polypeptide, or a gene encoding an effector polypeptide; and an Mmc3 ORF3 polypeptide, or a gene encoding an Mmc3 ORF3 polypeptide, where the guide RNA is engineered to target a nucleic acid molecule in the cell, and the guide RNA, effector polypeptide, and ORF3 polypeptide do not naturally occur together, where the target nucleic acid molecule is modified by the effector polypeptide. The effector polypeptide can be, for example, a Cpf1 effector or Mmc3 effector. The modification can be cleavage, or can be mutation, for example, by nucleotide changes, delection of nucleotides, or insertion of one or more nucleotide that may occur during repair processes following cleavage by the Mmc3 effector. In some embodiments, the method can further include delivering a donor or repair template sequence to the cell. The donor or repair sequence can optionally include sequences that mediate homologous recombination into the targeted locus, e.g., sequences having a homology to sequences at or proximal to the target site (protospacer) of the nucleic acid molecule. In some embodiments, the donor or repair fragment can include a selectable marker. In various embodiments, an Mmc3 ORF3 polypeptide can increase the efficiency of nucleic acid modification by an effector polypeptide.

The features of the invention are now described in illustrative embodiments in which certain principles of the invention are set forth. These particular embodiments are exemplary, and not intended to limit the scope of the invention.

### BRIEF DESCRIPTION OF THE DRAWINGS

FIG. 1 illustrates eight exemplary architectures of gene editing systems, showing arrangement of the related genes with respect to each other. A minimal system includes an effector polypeptide (Mmc3) and a CR (CRISPR) array. Only NoMmc3 and SfMmc3 systems show conservation of known cas1, cas2 and cas4 genes. ORF3 encodes a predicted protein of unknown function that is conserved across several Mmc3 systems.

FIG. 2A-2C shows an alignment of ORF3 protein sequences from Mmc3 systems. 1. No2Mmc3 ORF3, SEQ ID NO:55. 2. NoMmc3 ORF3, SEQ ID NO:53. 3. No3Mmc3 ORF3, SEQ ID NO:58. 4. Sfm ORF3, SEQ ID NO:50. 5. Sv2 ORF3, SEQ ID NO:56. 6. Sv3 ORF3, SEQ ID NO:57. 7. Sv ORF3, SEQ ID NO:51.

FIGS. 3A-3B, FIG. 3A shows an alignment of Mmc3 CRISPR repeat sequences, based on predictions from CRISPRfinder and CRISPRdetect software. The 3′ approsimately half of the sequence is highly conserved amongst Mmc3 systems. CRISPR repeat sequences shown are, top to bottom, Consensus: SEQ ID NO:27; SvMmc3: SEQ ID NO:30; SmpMmc3: SEQ ID NO:39; Smp2Mmc3: SEQ ID NO:40; ShMmc3: SEQ ID NO:32; SfpMmc3: SEQ ID NO:36; SfMmc3: SEQ ID NO:29; ObpMmc3: SEQ ID NO:42; NoMmc3: SEQ ID NO:33; NapMmc3: SEQ ID NO:31; CrpMmc3: SEQ ID NO:41; BdMmc3: SEQ ID NO:28. FIG. 3B depicts RNA secondary structure predictions for the conserved 3′ region of two Mmc3 family members SvMmc3 (SEQ ID NO:137) and BdMmc3 (SEQ ID NO:47).

FIG. 4 depicts the location of the RuvC I (light grey bars), RuvC II (dark grey bars), and RuvC III (black bars) catalytic sites (black bars) of the Ruv C domain in various CRISPR class 2 effector polypeptides. Mmc3 polypeptides have a unique RuvC sub-domain distribution in relation to other class 2 polypeptides, Cpf1, C2c1, C2c3, CasX, CasY (all Type V) and Cas9 (Type II). Scaling for each sub-type was derived using average lengths taken from the representative sequences shown in FIG. 5.

FIG. 5 shows an amino acid sequence alignment of RuvC catalytic motifs in Mmc3 effectors, with have unique spacing and sequence relative to other class 2 CRISPR systems. The numbers of residues between sequence blocks are listed along with the total number of residues in each protein. Conserved residues with amino acids with small side chains (G, S, T, C, A, V) are highlighted in white, with hydrophobic side chains (A, V, I, L, M, F, Y, W) are highlighted in light grey, with polar side chains (N, Q, H) are highlighted in grey, with negatively charged side chains (D, E) are highlighted in darker grey, and with positively charged side chains (R, K) are highlighted in darkest grey. Sequences shown are, top to bottom, BdMmc3: SEQ ID NO:1; SfpMmc3: SEQ ID NO:15; PcMmc3: SEQ ID NO:7; SfMmc3: SEQ ID NO:2; SvMmc3: SEQ ID NO:3; Sv2Mmc3: SEQ ID NO:17; ObpMmc3: SEQ ID NO:14; Smp2Mmc3: SEQ ID NO:12; NapMmc3: SEQ ID NO:4; SfpMmc3: SEQ ID NO:15; NoMmc3: SEQ ID NO:6; No2Mmc3: SEQ ID NO:16; ShMmc3: SEQ ID NO:5; SmpMmc3: SEQ ID NO:11; Smp3Mmc3: SEQ ID NO:10; CrpMmc3: SEQ ID NO:13.

FIG. 6 shows an amino acid sequence alignment of the Mmc3 zinc finger domain, where the conserved cysteines are marked above the alignment. Sequences shown are, top to bottom: BdMmc3: SEQ ID NO:1; SfpMmc3: SEQ ID NO:15; PcMmc3: SEQ ID NO:7; SfMmc3: SEQ ID NO:2; SvMmc3: SEQ ID NO:3; Sv2Mmc3: SEQ ID NO:17; ObpMmc3: SEQ ID NO:14; Smp2Mmc3: SEQ ID NO:12; NapMmc3: SEQ ID NO:4; SfpMmc3: SEQ ID NO:15; NoMmc3: SEQ ID NO:6; No2Mmc3: SEQ ID NO:16; ShMmc3: SEQ ID NO:5; SmpMmc3: SEQ ID NO:11; Smp3Mmc3: SEQ ID NO:10; CrpMmc3: SEQ ID NO:13.

FIG. 7 depicts the evolutionary relationship of Mmc3 effectors to other known Type V Effector proteins. Shown is a maximum-likelihood phylogenetic tree based on an amino acid alignment of all known Type V CRIPSR effector proteins using MUSCLE. Cpf1 sequences were taken from Zetsche et al. (2015) Cell 163: 759-771. C2c1 and C2c3 sequences were taken from Shmakov et al. 2015 Molecular Cell, 60(3), 385-397 and CasX and CasY sequences were taken from Burstein et al. 2017 Nature, 1-20. Bootstrap values were derived from 100 pseudoreplicates and show high support for Mmc3 as an evolutionary distinct Type V CRISPR system.

FIG. 8 illustrates a radial representation of the evolutionary tree, showing the relationship of Mmc3 with other Type V Effector families Labels for a subset of strains and high-support is recovered for Mmc3 representing a distinct monophyletic clade within Type V CRISPR systems.

FIG. 9 shows schematics of the depletion assay for quantifying targeted DNA cleavage activity of CRISPR/Cas and determining PAM preferences using a 6N PAM library.

FIG. 10 gives an overview of the workflow for quantifying targeted DNA cleavage activity of CRISPR/Cas and determining PAM preferences using a 6N PAM library.

FIG. 11 illustrates the vectors and components of plasmid depletion assays used to discover Mmc3 PAM preferences and demonstrate targeted DNA cleavage activity. Top: Low copy vector for expression of Effectors under control of the Ptet promoter. Middle: Low copy vector expressing a minimal synthetic CRISPR array composed on a spacer sequence (Spacer 1) flanked by system specific CRISPR repeat sequences. Bottom: Target plasmid encoding a protospacer matching Spacer 1 sequence flanked by a 5′ N6 PAM library, or specific PAM sequence.

FIGS. 12A-12D FIG. 12A) shows PAM enrichment scores represented as SeqLogos for the Mmc3 family member SvMmc3. A 5′ TTN sequence is indicated as the top predicted PAM and is consistent across both biological and technical replicates. B) shows PAM enrichment scores represented as SeqLogos for the Mmc3 family member SfMmc3. A 5′ TTN sequence is indicated the top predicted PAM and is consistent across both biological and technical replicates. C) shows PAM enrichment scores represented as SeqLogos for the Mmc3 family member NoMmc3. A 5′ CTN sequence is indicated as the top predicted PAM and is consistent across both biological and technical replicates. D) shows PAM enrichment scores represented as SeqLogos for BdMmc3. A 5′ CTN or 5′ TTN sequence is indicated as the top predicted PAM depending on biological replicate. Results are consistent between technical replicates.

FIG. 13 provides a schematic illustration of an assay to quantify targeted DNA cleavage activity of CRISPR/Cas systems using target plasmids with specific PAM sequences.

FIG. 14 illustrates PAM dependence of DNA interference activities for the following Mmc3 systems: BdMmc3, NoMmc3, SfMmc3 and SvMmc3. Mmc3 systems were assessed for DNA-interference activity by comparing transformation frequencies of target plasmids encoding the following 5′-PAM sequences flanking the targeted protospacer (Sp1): 1) 5′-TTTT 2) 5′-ATTC 3) 5′-ACTC 4) 5′-TATC 5) 5′-TCTC 6) 5′-GTTC 7) 5′ TTTC 8) 5′ GGGG. In addition, a non-targeted protospacer (Sp2) control was performed. Relative reduction in transformation frequency compared to non-target control indicates activity of system for RNA-guided DNA interference. From this analysis, BdMmc3 and NoMmc3 and SfMmc3 activity profile is consistent with a 5′-HTN PAM, whereas SfMmc3 activity profile us consistent with a 5′-TTV PAM.

FIG. 15 shows target specific DNA interference activities of the Mmc3 systems relative to AsCpf1: BdMmc3, NoMmc3, SfMmc3, SvMmc3 and AsCpf1. The designation “Correct Target” indicates plasmids which encode a protospacer that matches the guide RNA spacer sequence (Sp1), whereas the “Incorrect Target” plasmid encode a protospacer that is mismatched with the guide RNA spacer sequence (Sp2). The relative reduction in transformation frequency between “Correct” and “Incorrect” target experiments indicates activity of system for RNA-guided DNA interference. Both target plasmids encode the 5′ TTTC PAM sequence shown to support activity of Mmc3 systems and AsCpf1. From this analysis, all Mmc3 systems show 3-4 log reduction on transformation frequency for the correct target relative to the incorrect target. BdMmc3 and SfMmc3 are more active for DNA cutting in the E. coli bioassay relative to AsCpf1.

FIGS. 16A and 16B shows target specific DNA interference activities of the Mmc3 systems: NapMmc3, ShMmc3, PcMmc3, Smp3Mmc3, Smp3Mmc2, SfpMmc3, and ObpMmc3, CrpMmc3, SmpMmc3, and AsCpf1. The designation “Correct Target” indicates plasmids which encode a protospacer that matches the guide RNA spacer sequence (Sp1), whereas the “Incorrect Target” plasmid encode a protospacer that is mismatched with the guide RNA spacer sequence (Sp2). The relative reduction in transformation frequency between “Correct” and “Incorrect” target experiments indicates activity of system for RNA-guided DNA interference. Both target plasmids encode the 5′ TTTC PAM sequence shown to support activity of Mmc3 and AsCpf1 systems.

FIGS. 17A-17D depicts the results of RNAseq in determining the sequence of the processed guide RNA of four Mmc3 systems. Small RNA from cells expressing both an Mmc3 effector and a minimal CRIPSR array was purified and sequenced. Trimmed reads are shown mapped to the CRISPR array and report on guide RNA processing by Mmc3 systems.

FIGS. 17E-17G shows diagrams of constructs encoding various configurations of guide RNAs used to validate predictions for guide RNA processing by Mmc3. Activity associated with various guide RNA constructs was assessed with plasmid interference assays and are reported in bar graph form.

FIGS. 18A and 18B depicts a construct for expressing multiple processed guide RNAs from a single CRISPR array in the presence of an Mmc3 effector. The ability to cleave multiple targets utilizing these guide RNAs was validated using plasmid interference assays with different target plasmids design to hybridize with the different guide RNAs. Results are presented in bar graph form.

FIG. 19 is a diagram of the E. coli rpoB locus with the guide (spacer) sequences tested for ability to support targeted genomic cleavage by CRISPR effectors. Associated PAM sequences are also indicated.

FIGS. 20A-20D provides the results of chromosomal targeting assays using the BdMmc3 and NoMmc3 effectors as well as known Type V (AsCpf1) and Type II (SpCas9) effectors using guide RNAs having the spacer sequences diagramed in FIG. 19. Reductions in transformation frequency indicates lethality associated with cleavage of the E. coli chromosome.

FIGS. 21A and 21B provides a diagram of the repair plasmid that includes a repair fragment encoding mutations for rifR and ablation of the target site as well as a sequence encoding the guide RNA for targeting the rpoB locus in E. coli. Target site ablation and system specificity was validated by showing that a guide RNA encoding the mutations (rpoB-Sp2-EDIT) in the repair template did not support activity in the plasmid interference assay. Also tested were the original rpoB-Sp2 guide RNA, rpoB-sp2 guide RNA construct with REPAIR fragment and a non-targeting guide RNA control.

FIG. 22 provides the results of testing Mmc3 for CRISPR-assisted editing of the rpoB locus for using a repair fragment encoding mutations for RifR. Frequencies of RifR due to spontaneous mutation, recombination and CRISPR-assisted editing are shown for BdMmc3 and Cas9 relative to neg. controls without CRISPR effectors. BdMmc3 increases the effective frequency of RifR clones 3-4 orders of magnitude over recombination frequencies in the absence of Mmc3.

FIG. 23 shows alleles of representative RifR clones derived from the experiment in FIG. 22. Clones derived from populations harboring Mmc3 and the repair plasmid have the expected mutation spectrum, whereas clones from neg. controls show signatures of spontaneous mutation, or Wt sequence.

FIG. 24 shows the results of plasmid interference assays where particular amino acids of the RuvC motifs of the BdMmc3 effector were mutated to alanine. An AsCpf1 ruvC mutant is also shown as a positive control.

FIG. 25 is a schematic diagram of the beta-galactosidase assay for measuring repression of transcriptional activity by nuclease deficient Mmc3 effectors mutated in the catalytic domain

FIGS. 26A-26D are graphs showing the reduction in LacZ (beta-galactosidase) activity, measured as absorbance at 420 nm, of E. coli expressing nuclease-deficient mutants dAsCpf1 (A and C) and dBdMmc3 (B and D) that were co-expressed with guide RNAs targeting LacI and LacZ genes in E. coli.

FIG. 27 shows the results of plasmid interference assays where cysteine residues of the zinc finger domain of Mmc3 effectors SfMmc3 and NoMmc3 were mutated to alanine.

FIG. 28 is a schematic diagram of the assay used to detect Mmc3-mediated nucleic acid modification in yeast, where the guide RNA was delivered into cells expressing the Mmc3 effector.

FIG. 29 provides bar graphs of the assay used to targeted nucleic acid modification in yeast cells expressing the BdMmc3 effector and the NoMmc3 effector as well as control Type II (AsCpf1) and Type V (SpCas9) effectors.

FIG. 30 provides bar graphs of the assay used to targeted nucleic acid modification in yeast cells expressing the BdMmc3 effector, the NoMmc3 effector, the Smp2Mmc3 effector, the ShMmc3 effector, the SfMmc3 effector, and the SfpMmc3 effector, as well as control Type II (AsCpf1) and Type V (SpCas9) effectors.

FIGS. 31A-31D depicts the assay used to detect Mmc3-mediated nucleic acid modification in yeast cells. A) is a schematic diagram of a components for testing chromosomal editing with Mmc3 effectors in yeast using exogenously supplied guide RNA. Yeast expressing the Mmc3 effector is transformed with two in vitro transcribed guide RNAs, a repair template and a transformation selection plasmid. B) is a schematic diagram showing Mmc3-dependent cleavage at chromosomal sites T1 and T3 is repaired by homologous recombination to generate an ˜200 bp deletion that is subsequently detected by PCR. C) Provides gels showing the results of chromosomal editing in S. cerevisiae with the BdMmc3 effector and exogenous guide RNA. Colony PCR spanning the predicted cut sites gives product about ˜200 smaller when repaired by homologous recombination with repair template. Top gels, control: Cells transformed with only dsDNA repair fragment and no guide RNA do not show evidence for introduction of the deletion by homologous recombination. Bottom gels, guide RNA dependent editing: Cells transformed with both guide RNA and repair template show 3/96 clones with the predicted ˜200 bp deletion demonstrating BdMmc3-dependent editing of the yeast chromosome. D) Sequencing confirmation of BdMmc3-dependent editing. PCR products from 31C (H11, A05, B03 and - Ve control) were sequenced and aligned to the S. cerevisiae genome. All three edited clones show the correct deletion predicted by the homologous recombination with the repair template, whereas the negative control showed the wild type sequence.

FIGS. 32A-32B, FIG. 32A) is a schematic diagram of the protocol for testing chromosomal editing with Mmc3 in yeast using in vivo expressed guide RNA to generate a deletion at chromosomal cut site T3. B) provides photographs of gels showing chromosomal editing in S. cerevisiae with BdMmc3 and in vivo expressed guide RNA. Colony PCR spanning the predicted cut sites gives product approximately 200 bp smaller when repaired by homologous recombination with the repair template. Upper gel: cells transformed with BdMmc3 guide RNA and repair template show 18 of 22 clones with the predicted deletion. Lower gel: cells transformed with non-cognate Cas9 guide RNA and repair template show only bands of wild type size.

FIGS. 33A-33B, FIG. 33A) is a schematic diagram of the protocol for testing chromosomal editing with Mmc3 in yeast using in vivo expressed guide RNA to generate an insertion at chromosomal cut site T3. B) is a gel for analyzing chromosomal editing in S. cerevisiae with BdMmc3 and in vivo expressed guide RNA. Colony PCR spanning the predicted cut sites gives product about 700 bp larger when repaired by homologous recombination with repair template encoding insertion. (Top, experiment) Cells transformed with BdMmc3 guide RNA and repair template show 4/19 clones with predicted insertion. (Bottom, control) Cells transformed with non-cognate Cas9 sgRNA and repair template show only wild type sequence.

FIG. 34 is a schematic of a protocol to test chromosomal editing with Mmc3 in mammalian cells. Cells expressing a cell surface marker, such as CD46, are transfected with a plasmid that expressed Mmc3 as a 2a-GFP fusion and a cognate guide RNA array targeting the cell surface marker gene. Targeted disruption of the cell surface marker gene, results in loss of the marker from the cell surface as a function of growth. FACS is used to identify edited cells that both express Mmc3 (GFP+) and have lost the cell surface marker (CD46 −).

FIG. 35 is a diagram showing guide RNA processing by Mmc3 in mammalian cells demonstrated by RNAseq. Small RNA was purified from cells expressing an Mmc3 effector and guide RNA array. Sequencing and alignment to the guide RNA template showed evidence of guide RNA processing for, SfMmc3, NoMmc3 and BdMmc3. The arrows indicate the 5′ processing site that results in a 18-19 nt CR repeat instead of the full length 36 bp repeat encoded in the guide RNA array. Mmc3s were able to process the 3′ end of the guide RNA array to yield a spacer sequence typically ranging from 20 to 28 nt. BdMmc3 demosntrates more restricted processing of the 3′ end of the guide RNA consistent with results from E. coli RNAseq analysis of BdMmc3 guide RNA.

FIG. 36A-36B, FIG. 36A) provides a diagram of the construct used to express a guide RNA in the alga Nannochloropsis. Ribozyme sequences in the construct catalyze cleavage into a “processed” guide seqeunce. B) provides the construct for expressing the BdMmc3 effector in Nannochloropsis along with the BSD selectable marker, a guide RNA, and GFP.

FIG. 37 is a photograph of a gel showing cutting of a plasmid that includes a target sequence in a mammalian cell lysate by the AsCpf1 and Smp2Cpf1 effectors in the presence and absence of the Mmc3 ORF3 polypeptide.

FIG. 38 is a photograph of a gel showing a time course of digestion by Smp2Cpf1 effector produced in a cell lysate with ORF3 polypeptide added to the assay (left half of photograph) and without ORF3 polypeptide added to the assay (right half of photograph).

FIG. 39 is a photograph of a gel separating products of 30 minute assays of AsCpf1 and Smp2Cpf1, with and without added Mmc3 ORF3 polypeptide. Analysis of band intensities provides that the presence of the ORF3 polypeptide resulted in 1.6 fold the control (no ORF3 polypeptide present) amount of cutting when AsCpf1 was used as the effector, and 9-fold the control level of cutting when Smp2Cpf1 was the effector.

### DETAILED DESCRIPTION OF THE INVENTION

Polypeptides having nucleic acid cleavage activity in prokaryotic and eukaryotic cells are disclosed herein. These polypeptides are useful in engineered CRISPR-Cas systems where they can generate programmable double stranded breaks (DSBs) at defined locations in a target nucleic acid sequence, either in vivo or in vitro. Upon generation of DSBs at specific sites within the genome in a living cell, cellular DNA repair mechanisms can act on the cleaved genomic sequences which provides for in situ gene editing. The nucleases described herein are components of a new family of Class 2 Type V CRISPR systems referred to as Mmc3.

CRISPR/Cas systems are generated using Mmc3 family nucleases and are expressed in a living cell, where the expressed components (namely the guide RNA and any associated Cas proteins, including the effector nuclease) are non-naturally occurring entities introduced into the cell as DNA for a certain specific gene altering function to be achieved. Typically for engineered use, Cas genes other than the effector are not required for activity. The elements of this system are designed as custom, ex-vivo, and highly specific elements, where a spacer sequence (or guide sequence) of choice is encoded as a guide RNA, where the guide RNA has a desirable secondary structure and can effectively bind to the target DNA, the spacer or guide sequence having complementarity with a corresponding sequence in the genome or episomal DNA of the organism where the CRISPR/Cas system is introduced, and can also bind to the Cas nuclease to direct the designated DNA cleavage. The guide RNA may be processed and may further contain at least a partial repeat sequence. The Mmc3 nuclease effectively and efficiently cleaves the genome of the host cell in which it is expressed at the predetermined region as guided and specified by the guide RNA. Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All ranges provided within the application are inclusive of the values of the upper and lower ends of the range unless specifically indicated otherwise.

All publications, patents and other references mentioned herein are incorporated by reference in their entireties for all purposes as if each individual publication or patent application were specifically and individually indicated to be incorporated by reference.

The term “and/or” as used in a phrase such as “A and/or B” herein is intended to include “A and B”, “A or B”, “A”, and “B”.

“About” means either within 10% of the stated value, or within 5% of the stated value, or in some cases within 2.5% of the stated value, or, “about” can mean rounded to the nearest significant digit.

The term “guide RNA” refers to a polynucleotide sequence having a guide or spacer sequence (which may also be referred to as a targeting sequence), that has sufficient complementarity with a target nucleic acid sequence, to hybridize with the target nucleic acid sequence and direct sequence-specific binding of a nucleic acid-targeting complex to the target nucleic acid sequence. A guide sequence of a guide RNA is typically 18-25 nucleotides in length. A guide RNA also includes a sequence or sequences that interact with the effector protein that are derived from repeat sequences of CRISPR arrays (referred to as “CRISPR repeat sequences” or “repeat seqences”), but as used herein a guide RNA does not include tracr sequences—i.e., does not include sequences derived from tracr RNAs that are components of some CRISPR systems.

The invention provides a method for altering gene expression in a living cell, a eukaryotic cell, a prokaryotic cell, a cell in culture, or a cell within an organism, where the organism is a prokaryotic or eukaryotic organism, and can be, for example, an animal, plant, alga, labyrinthulomycete, or fungus. The Mmc3 nuclease system expands the potential of current CRISPR-based gene editing platforms, providing several advantages. For example, the Mmc3 family proteins are smaller in size compared to many other Type V effectors, which simplifies transfection, stability and expression. Additionally, some Mmc3 effectors have a more relaxed PAM requirement than the systems currently in use. For example, NoMmc3, BdMmc3 and SfMmc3 can accept an A, C or T in the third position (relative to the junction with the protospacer), whereas AsCpf1 and LbCpf1 are reported to require a T (Kim et al. 2017). NoMmc3, BdMmc3 and SfMmc3 can accept a T at the first position, whereas AsCpf1 and LbCpf1 cannot (Kim et al 2017). Additionally, some Mmc3 effectors only require a 3 bp PAM, instead of a 4 bp PAM like AsCpf1 and LbCpf1. In general, a more relaxed PAM sequence can be useful as it may allow more sites to be targeted per genome. Mmc3 CRISPR-Cas Systems

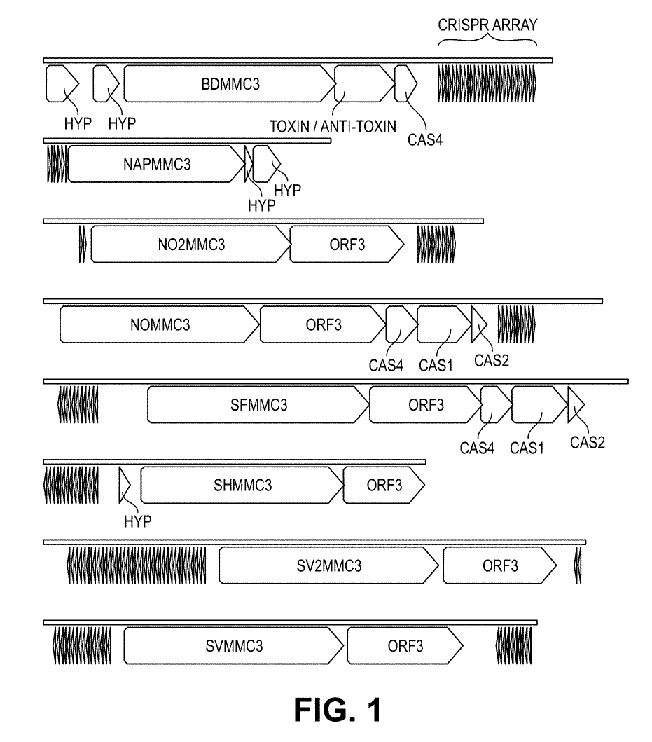

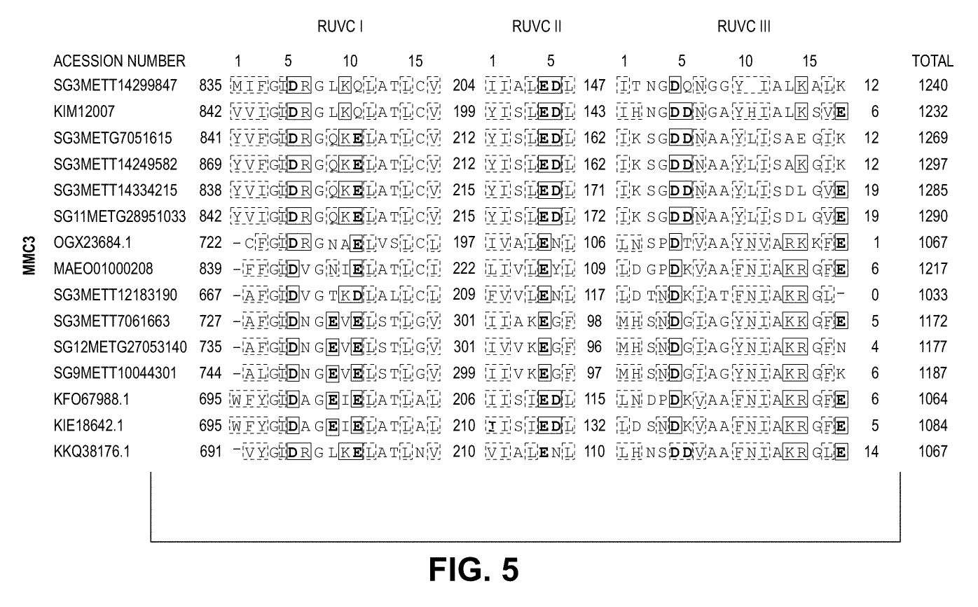

FIG. 1 shows the basic arrangement of genes and sequences for exemplary Mmc3 CRISPR loci. As shown, these loci can exhibit a variety of different architectures. The minimal structure includes an effector gene (Mmc3) and a CRISPR (CR) array. Several of the Mmc3 CRISPR systems (e.g., NoMmc3 and SfMmc3) encode Cas4, Cas1, and Cas2 genes; however the majority do not. The presence of Cas 4, Cas 1 and Cas 2 as accessory proteins is conserved among Type V CRISPR systems (see, Table 1). ORF 3 is a gene found only in Mmc3 systems to date; it is however not universally present in Mmc3 CRISPR systems. Also evident from Table 1 is the large overall amino acid sequence divergence in the effector polypeptides among the various Type V members. Upon comparison, the Cpf1 family (as represented by the members whose sequences are aligned in FIG. 5A) shows 18-43% sequence identity to a well characterized Cpf1 family member, FnCpf1. This range is higher (32-40% identity) if only active Cpf1 effectors are considered (See Zetsche et al. 2015). On the other hand Mmc3 members exhibit only about 8% to 11% identity with FnCpf1, which is comparable to the degree of identity between FnCpf1 and C2C3, CasX and CasY subtypes (Table 1).

TABLE 1
Comparison of Mmc3 to known Type V CRISPR systems

| | | | Cas | Aux. | | Effector | ID to |
|---|---|---|---|---|---|---|---|
| Sub-Type | tracr | RuvC | genes | Genes^{#} | PAM | size (aa) | FnCpf1 |
| Cpf1 | No | Yes | Cas4, 1, 2 | None | 5′ TTN/ | 1206-1373* | 32-40%** |
| | | | | | TTTN | | |
| C2c1 | Yes | Yes | Cas4-1, 2 | None | 5′ TTN/ | 1100-1500 | 6-8% |
| | | | | | ATTN | | |
| C2c3 | ? | Yes | Cas1* | None | ? | 1200-1300 | 8-10% |
| CasX | Yes | Yes | Cas4, 1, 2 | None | 5′ TTCN | 980 | 11-12% |
| CasY | No | Yes | Cas1* | None | 5′ TA | ~1200 | 7-10% |
| Mmc3 | No | Yes | Cas4, 1, 2* | Putative | 5′ HTN/ | 1033-1297 | 8-11% |
| | | | | | TTV | | |

*Not all systems have the canonical Cas gene architecture
**Based on systems demonstrated as functional by Zetsche et al 2015.
^{#}Defined as genes and their products that modify the DSB activity of the effector

Some CRISPR-Cas systems are dependent on an auxiliary RNA called traguide RNA, which is needed to facilitate the formation of Cas complexes with the guide RNA and bringing about the DNA cleavage, whereas other systems are traguide RNA independent. Cas9, C2c1, and CasX require an auxiliary traguide RNA for correct processing and loading of guide RNA into the effector protein. Mmc3 systems were assessed for the presence of traguide RNA by analyzing intergenic regions for sequence with partial complementarity to CRISPR repeat sequences. Suboptimal alignments failed to uncover strong evidence for an anti-repeat sequence typical of traguide RNA from other systems (i.e. Cas9, C2c1). In addition, assays of engineered Mmc3 systems (Examples 2, 3, 5, 6, 7, 8) demonstrate the lack of requirement for a traguide RNA. The Mmc3 family of nucleases are thus traguide RNA independent.

Among the various effector proteins used in CRISPR systems, Class 1 Cas systems comprise multiple subunit effector molecules. Class 2 effector proteins on the other hand, comprise single protein effector complex. Numerous Class 2 CRISPR systems have been described, including Cas9, Cpf1, C2c1, CasX and CasY. These Class 2 systems are defined minimally by i) a single large effector protein responsible for cleaving the target DNA, ii) a CRISPR array encoding targeting information. The Mmc3 family of nucleases are single protein effectors and would be considered a Class 2 system. The uniqueness of the effector protein has been a dominant criterion used by the field to classify and distinguish different CRISPR systems (See Zetsche et al. 2015, Shmakov et al 2015, Shmakov et al 2017 and Burstein et al 2017). According to the present classification, Class 1 CRISPR effectors are exemplified by Type I, III and IV systems. Class 2 CRISPR Effectors are of three distinct types: Type II and Type V systems target DNA, and Type VI systems target RNA. Cas9 would be exemplary of a Type II system, while Cpf1, C2c1, C2c3, Cas X and Cas Y are exemplary of Type V system members. The Mmc3 family of nucleases are Type V members.

Type V CRISPR effectors are typified by a C-terminal RuvC domain derived from transposon ORF-B, and the absence of an HNH domain seen in Type II (Cas9) enzymes. Cpf1 was the first Type V enzyme described and was unique relative to Cas9, given its use of a single guide RNA, that is, its function is independent of traguide RNA, its ability to process its own guide RNA, its generation of a staggered cut in the target DNA, and its requirement for a ‘T-rich’ PAM sequence. Additional Type V systems (C2c1, C2c3, CasX, CasY) are generally consistent with this description of Cpf1, although these systems are not as well characterized at the functional level. Other characteristics of Type V systems have been less generalizable. For instance, PAM sequences differ amongst Type V systems both within and between sub-types but in general are 5′ PAMs that are AT-rich, which is distinct from Type II (Cas9) PAMs which are 3′ G-rich sequences. CRISPR array repeat sequences can also differ markedly within and between subtypes.

The Mmc3 family of nucleases are generally characterized by the presence of a noncontiguous RuvC catalytic domain, the absence of a HNH domain, the absence of a nuc domain, the lack of a requirement for tracr RNA, an affinity for T-rich PAM sequences, and the presence of a zinc finger domain characterized by two cysteine pairs located between RuvC motifs II and III. Of particular note is the unique sequences and spacing of the RuvC I, II and III sequences of the Mmc3 family Table 4 shows Mmc3 effectors have unique consensus sequences across all three RuvC sub-domains in comparison to other known effector proteins. Consensus sequence of class 2 CRISPR Effectors, consisting of RuvC catalytic motifs and surrounding residues, were derived from the representative sequences of each sub-type listed in FIG. 5A and FIG. 5B.

In addition to having a very low overall sequence identify to other known Type V effector proteins, the Mmc3 family effector proteins are characteristically smaller in size compared to many other effectors. Cas9 proteins are typically larger than Type V Effectors with median length of ˜1350 aa (based on reference sequences compared in FIG. 5).

### Mmc3 Effector Proteins

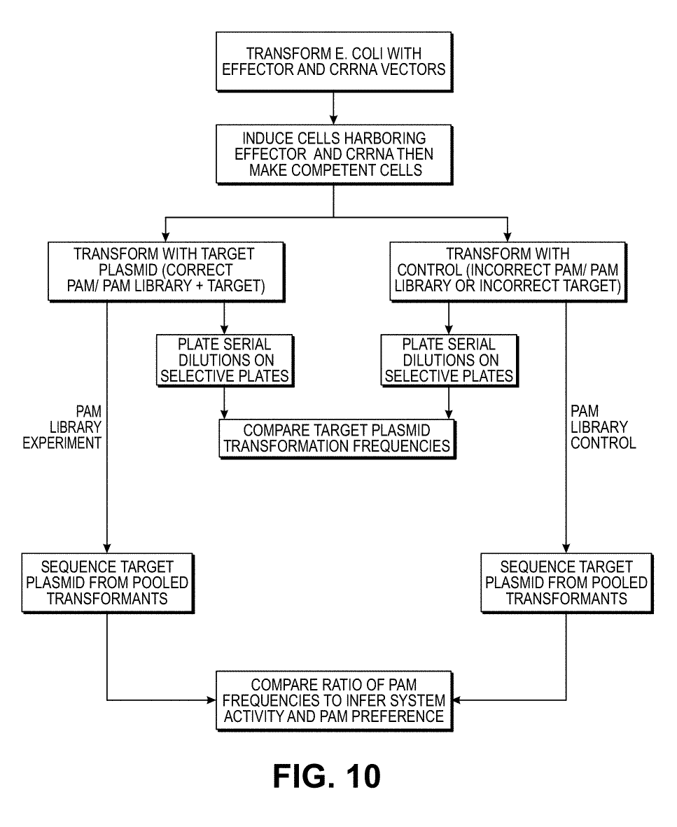

Mmc3 CRISPR systems include Mmc3 effector proteins or genes encoding Mmc3 effector proteins, where “Mmc3 effector protein” “Mmc3 effector” “Mmc3 RNA-guided nuclease” “Mmc3 nuclease” or in some cases “Mmc3 polypeptide” are all used herein to refer to the RNA-guided endonuclease cas protein of a naturally-occurring Class 2, Type V Mmc3 CRISPR system and variants thereof. As described in detail in Example 1, the Mmc3 family of RNA-guided nucleases is demonstrated by bioinformatic analysis to form a distinct family within the Class 2 Type V CRISPR effectors. For example, the all-by-all blast analysis described in Example 1 and depicted diagramatically in FIG. 10 shows that members of the Mmc3 family cluster together on the basis of sequence homology. Mmc3 effectors are tracr-independent: when complexed with a guide RNA that includes a guide sequence and sequences derived from CRISPR array repeat sequences an Mmc3 effector cleaves double-stranded DNA in the absence of a traguide RNA or a tracr sequence.

Mmc3 effectors include a noncontiguous RuvC domain that comprises three RuvC motifs and do not include an HNH domain (present in Cas9, a Type II effector) or a nuc domain (characteristic of Cpf1, a Type V effector), but do include a zinc finger domain having four cysteine residues (see Example 1).

The spacing of the three noncontiguous RuvC motifs of the RuvC domain of Mmc3 effectors is distinctive, where the RuvC I motif and RuvC II motif are separated by more that 125 amino acids and the RuvCII motif and RuvCIII motif are separated by fewer than 225 amino acids. The spacing between RuvC I and RuvC II of Mmc3 effectors ranges from 125 amino acids to about 350 amino acids, and the spacing between RuvC II and RuvC III of Mmc3 effectors ranges from about 25 amino acids to 225 amino acids. For example, the spacing between RuvC I and RuvC II can range from 150 amino acids to about 325 amino acids or from 175 amino acids to about 300 amino acids, and the spacing between RuvC II and RuvC III can range from about 50 amino acids to 200 amino acids or from about 75 amino acids to 175 amino acids. This spacing of RuvC motifs is different from that of Cpf1 effectors, for example, that have a shorter spacing between motifs I and II and a longer spacing between motifs II and III, and C2c1, which has a longer spacing between motifs I and II (see Table 5).

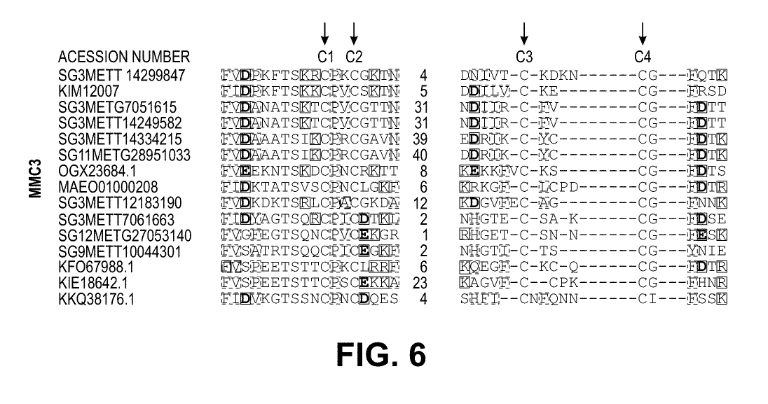

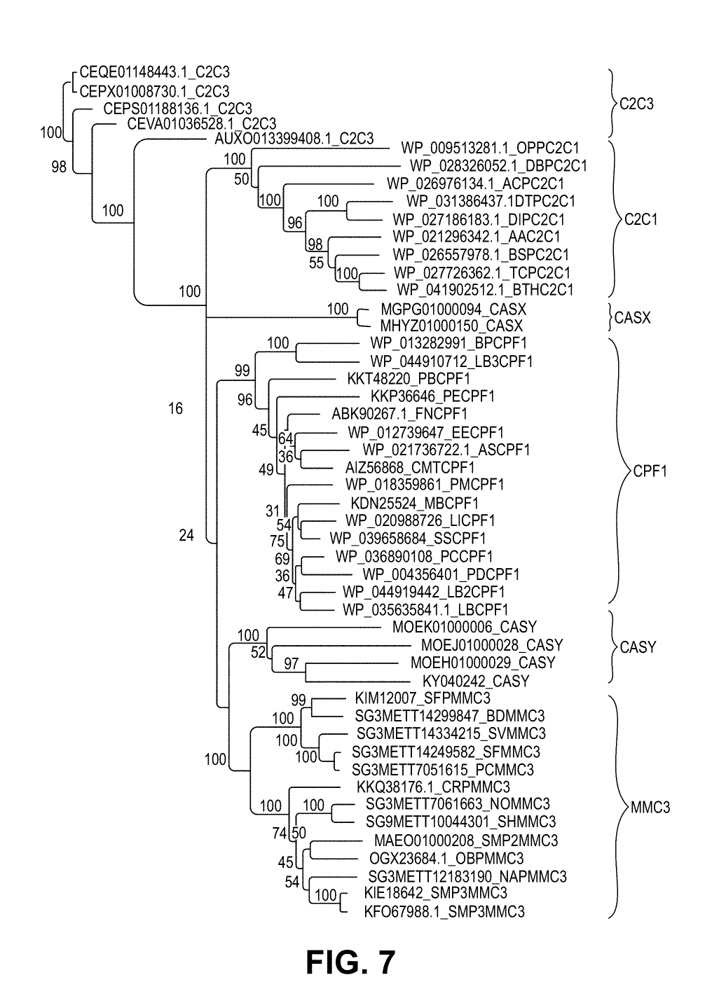

Mmc3 effectors also have a zinc finger domain that is positioned between the RuvCII and RuvCIII motifs of Mmc3 effector proteins. This domain is characterized by two pairs of cysteine residues, where the cysteine residues of the first pair (referred to herein as the first and second cysteine residues) are separated by two intervening amino acids and the cysteine residues of the second pair (referred to herein as the third and fourth cysteine residues) are separated by between two and five intervening amino acids (FIGS. 7A and 7B). There are several conserved residues in the vicinity of the cysteine pairs, for example, there is a phenylalaline residue at position “−10” with respect to the first cysteine; a valine or isoleucine at position “−9” with respect to the first cysteine; and the amino acid immediately following (C-terminal to) the first cysteine is proline (P) and/or the two amino acids at positions “−4” and “−3” are threonine and serine, respectively. As disclosed in Example 7, mutation of the cysteine residues of the first cysteine pair to alanine abolishes Mmc3 effector activity.

In various embodiments of engineered or non-naturally occurring CRISPR-Cas systems provided herein the Mmc3 effector polypeptide can comprise:

an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or a variant of an Mmc3 effector comprising an amino acid sequence having at least 60%, 65%, 70%, 75%, 80%, 85%, 90%, or 95% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or

a naturally-occurring Mmc3 effector comprising an amino acid sequence having at least 50%, 55%, 60%, 65%, 70%, 75%, 80%, 85%, 90%, or 95% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26.

For example, an engineered or non-naturally occurring CRISPR-Cas system provided herein the Mmc3 effector polypeptide can comprise:

an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:12, SEQ ID NO:16, SEQ ID NO:17, SEQ ID NO:20, SEQ ID NO:23, SEQ ID NO:24, and SEQ ID NO:25; or

a variant of an Mmc3 effector comprising an amino acid sequence having at least 60%, 65%, 70%, 75%, 80%, 85%, 90%, or 95% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:16, SEQ ID NO:17, SEQ ID NO:20, SEQ ID NO:23, SEQ ID NO:24, or SEQ ID NO:25; or

a naturally-occurring Mmc3 effector having at least 50%, 55%, 60%, 65%, 70%, 75%, 80%, 85%, 90%, or 95% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:16, SEQ ID NO:17, SEQ ID NO:20, SEQ ID NO:23, SEQ ID NO:24, or SEQ ID NO:25.

In further examples, an engineered or non-naturally occurring CRISPR-Cas systems provided herein the Mmc3 effector polypeptide can comprise:

an amino acid sequence selected from the group consisting of SEQ ID NO:1, SEQ ID NO:2, SEQ ID NO:3, SEQ ID NO:5, SEQ ID NO:6, and SEQ ID NO:7; or

a variant of an Mmc3 effector comprising an amino acid sequence having at least 60%, 65%, 70%, 75%, 80%, 85%, 90%, or 95% identity to SEQ ID NO:1, SEQ ID NO:2, SEQ ID NO:3, SEQ ID NO:5, SEQ ID NO:6, or SEQ ID NO:7; or

a naturally-occurring Mmc3 effector having at least 50%, 60%, 65%, 70%, 75%, 80%, 85%, 90%, or 95% identity to SEQ ID NO:1, SEQ ID NO:2, SEQ ID NO:3, SEQ ID NO:5, SEQ ID NO:6, or SEQ ID NO:7.

### Guide RNAs

The term “guide RNA” refers to an RNA that includes a guide sequence that has homology with a target sequence (sometimes referred to as a “protospacer”, where the guide sequence can be referrred to as the “spacer”) and additional sequence that allows for interaction of the guide RNA with the effector protein, which may be referred to as the “handle” of the guide, and is derived from CRISPR repeats, so may also be referred to as the repeat sequence of the guide. As used herein, the term guide RNA encompasses guide RNAs (plural). In the CRISPR systems provided herein, a guide RNA may not include tracr sequences. Some CRISPR systems require tracrRNA sequences, but the Mmc3 and Cpf1 systems disclosed herein are tracr-independen. (A guide RNA that does include tracr systems may be referred to as a “chimeric guide RNA” or a “single guide RNA” or “sgRNA”.) The degree of complementarity between a guide sequence and a target sequence, when optimally aligned using a suitable alignment algorithm, may vary and is commonly at least 50%, 60%, 75%, 80%, 85%, 90%, 95%, 97.5%, 99%, or is about 100% identical to the target sequence. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a target cell having the corresponding target nucleic acid sequence, such as by transfection with vectors encoding the components of the nucleic acid-targeting complex, followed by an assessment of preferential targeting (e.g., cleavage) within the target nucleic acid sequence. Similarly, cleavage of a target nucleic acid sequence may be evaluated in vitro by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide RNA may be selected to target any target nucleic acid sequence. The target sequence may be DNA or RNA such as messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (IncRNA), and small cytoplasmatic RNA (scRNA).

A guide RNA can include a repeat sequence that can be between about 15 and 50 nt in length, more commonly between about 16 and about 40 nt in length, and may be between about 17 nt and about 38 nt in length for some exemplary Mmc3 systems. The repeat sequence in the Mmc3 CRISPR systems disclosed herein is followed by (is 5′ to) the guide, or spacer, sequence that may be between about 17 and 35 nt in length, more typically between about 17 and about 30 nt in length, or between about 18 and about 25 nt in length. A guide construct can be derived from or designed to replicate the organization of a CRISPR array (CRarray), where a spacer is flanked by repeat sequences. In some examples, a CRarray construct can be engineered to have two or more spacers (guide sequences) that are typically of between about 17 and about 25 nt in length that are separated by CRISPR repeat sequences from about 15 to about 50 nucleotides in length. Alternatively a guide RNA construct can encode a “processed guide”, where the construct includes a repeat sequence of that may be from about 15 to about 30 nucleotides in length, for example, from about 17 to about 28 nucleotides in length, followed by a guide sequence that may be between about 15 and about 35 nt in length, more typically between about 17 and about 25 nt in length. CRISPR systems as provided herein can in some embodiments include constructs for expressing a guide RNA in a host cell that include multiplex guide constructs, where a CRarray includes two or more different spacer sequences. In some alternative embodiments, CRISPR systems as provided herein can include multiple guide RNAs that can target different sites that can be introduced into a target cell.

### ORF3 Polypeptides

As disclosed herein, ORF3 polypeptides are encoded by an open reading frame associated with several Mmc3 CRISPR loci (see, for example, FIG. 1). Without limiting the invention to any particular mechanism, ORF3 polypeptides may enhance the activity of a CRISPR effector, such as but not limited to an Mmc3 effector or a Cpf1 effector. For example, inclusion of an Mmc3 ORF3 polypeptide or a nucleotide sequence encoding an Mmc3 ORF3 polypeptide in a CRISPR-Cas system, such as but not limited to an Mmc3 CRISPR-Cas system or a Cpf1 CRISPR-Cas system, can result in an enhanced rate of DNA modification by the Mmc3 or Cpf1 CRISPR-Cas system. A Cpf1 effector used with an Mmc3 ORF3 polypeptide can be any Cpf1 effector or variant thereof, such as any descibed in US 2016/0208243 and US 2017/0233756, both of which are incorporated herein by reference. Exemplary Cpf1 effectors that may be used for modifying a target nucleic acid molecule in a system that includes an Mmc3 ORF3 polypeptide are the AsCpf1 effector (SEQ ID NO:81) and the Smp2Cpf1 effector (SEQ ID NO:200).

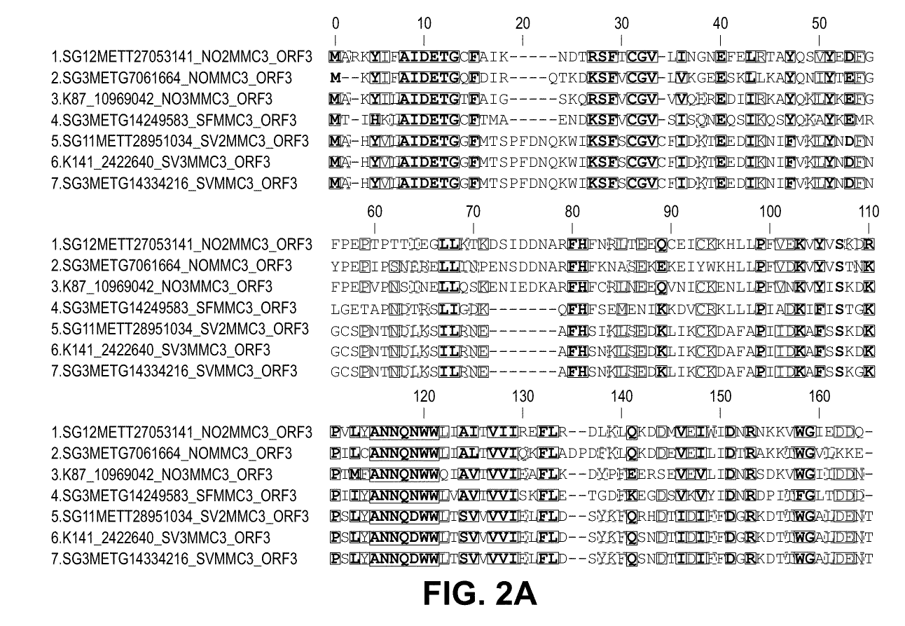

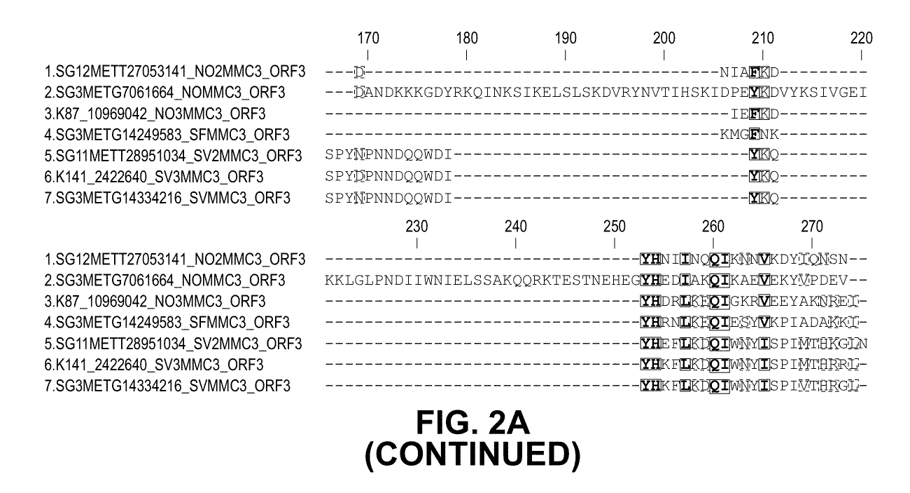

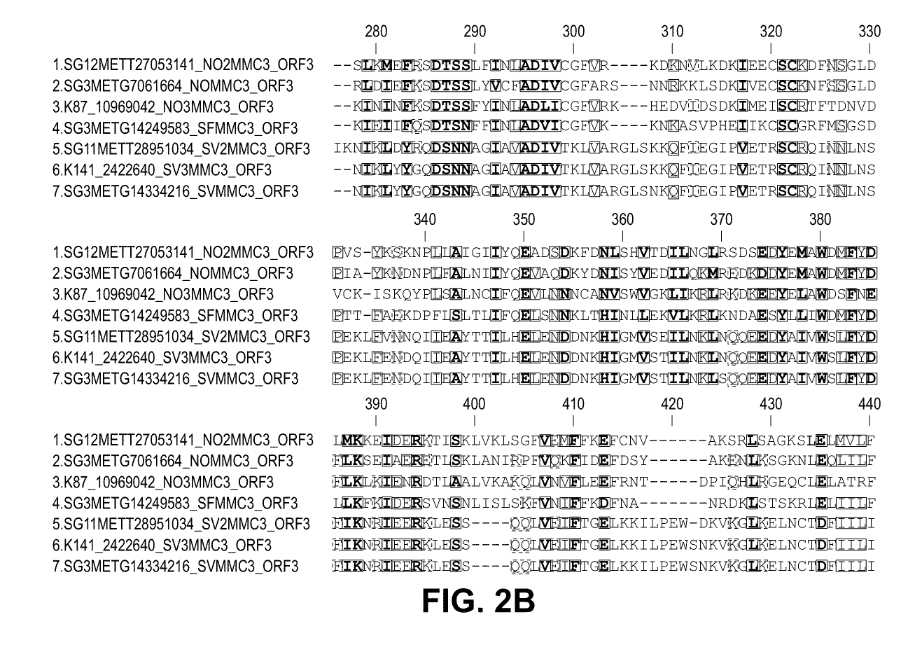

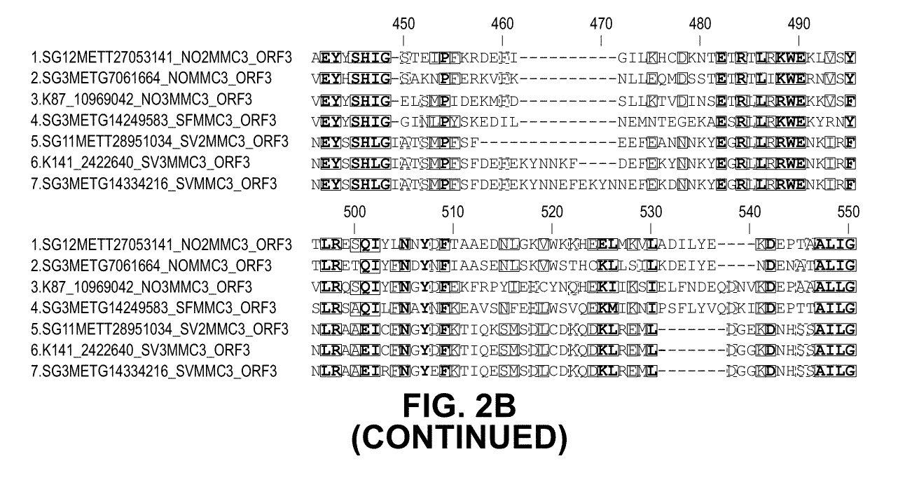

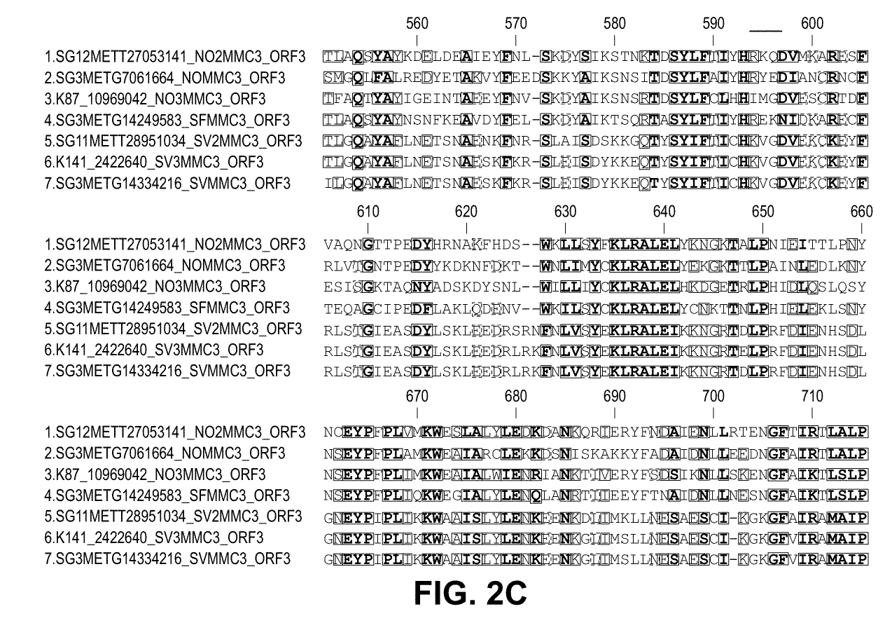

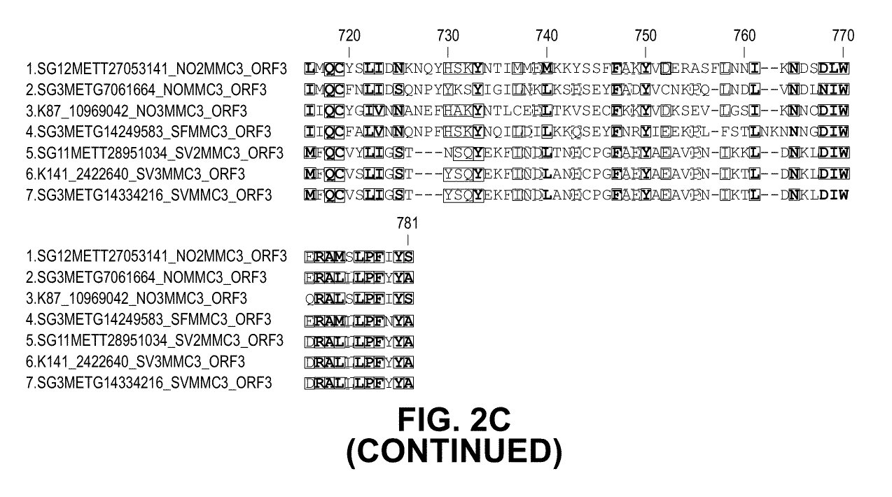

An Mmc3 ORF3 polypeptide used in any of the compositions and methods disclosed herein, including an ORF3 polypeptide encoded by an ORF3 gene used in any of the methods and compositions disclosed herein, can be any Mmc3 ORF3 polypeptide, e.g., any ORF3 polypeptide encoded by an ORF3 gene associated with an Mmc3 locus (see, for example, FIG. 1 providing the organization of several Mmc3 CRISPR loci, including the No2Mmc3, NoMmc3, SfMmc3. ShMmc3, Sv2Mmc3, and SvMmc3 loci, where ORF3 is proximal to (and downstream of) the Mmc3 effector gene). An Mmc3 ORF3 gene can be further be identified by relatedness of the encoded polypeptide to the ORF3 polypeptide sequences disclosed herein. For example, an ORF3 polypeptide encoded by an ORF3 gene of an Mmc3 locus can have at least 30%, at least 35%, at least 40%, at least 45%, at least 50%, at least 55%, at least 60%, at least 65%, at least 70%, at least 75%, at least 80%, at least 85%, at least 90%, or at least 95% amino acid sequence identity to an ORF3 polypeptide sequence such as any disclosed herein (e.g., an Mmc3 ORF3 polypeptide that comprises an amino acid sequence of SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58). In additional examples, an ORF3 polypeptide can include an amino acid sequence having at least 60%, at least 65%, at least 70%, at least 75%, at least 80%, at least 85%, at least 90%, at least 95%, at least 96%, at least 97%, at least 98%, at least 99% identity to a naturally-occurring ORF3 polypeptide, such as but not limited to any disclosed herein, for example, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58. FIG. 2A-C provides an alignment of full-length ORF3 polypeptide sequences of the No2, No, No3, Sf, Sv2, Sv3, and Sv mMc3 CRISPR loci. One of skill in the art could readily see areas where the identity of amino acids is highly conserved, less conserved, or not conserved and therefore be guided in generating or testing any variants. In some embodiments, an ORF3 polypeptide used in the compositions and/or methods provided herein can include an ORF3 polypeptide comprising an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% identity to any of SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58.

An ORF3 polypeptide, or a polynucleotide sequence encoding an ORF3 polypeptide, can be included in any CRISPR Cas system, including any of those disclosed herein. In various embodiments for in vivo editing, a cell may be engineered to express an Mmc3 ORF3 polypeptide, for example, may include a polynucleotide sequence encoding an Mmc3 polypeptide operably linked to a regulatory element, such as a promoter.

As demonstrated herein, an Mmc3 ORF3 polypeptide (or gene encoding an Mmc3 ORF3 polypeptide) used in the systems and methods provided herein can be an ORF3 polypeptide (or a gene encoding an ORF3 polypeptide) derived from a different CRISPR locus or can be derived from a different species from the CRISPR locus or species the effector protein or effector protein gene used in the systems or methods is derived from. For example, a CRISPR-Cas system as provided herein can include a gene encoding an Mmc3 effector and a gene encoding an Mmc3 ORF3 polypeptide, wherein the Mmc3 effector and Mmc3 ORF3 polypeptide are derived from different CRISPR loci of the same or a related species, or the Mmc3 effector and Mmc3 ORF3 polypeptide can be derived from different species, that may be, for example, derived from species of different genera. Further, an ORF3 gene or polypeptide can be used with a non-Mmc3 CRISPR effector, such as, for example, a Cpf1 effector. In some embodiments, an engineered, non-natural CRISPR-Cas system includes: an engineered or non-naturally-occurring CRISPR-Cas system having a polynucleotide sequence that encodes an engineered guide RNA comprising a guide sequence operably linked to a regulatory element, and an Mmc3 effector or a nucleic acid molecule encoding an Mmc3 effector operably linked to a regulatory element, and further includes an Mmc3 ORF3 polypeptide or or a nucleic acid molecule encoding an Mmc3 ORF3 polypeptide operably linked to a regulatory element.

### Engineered Mmc3 CRISPR-Cas Systems

An engineered or non-naturally-occurring CRISPR gene editing system as provided herein includes a guide RNA or a nucleotide sequence encoding a guide RNA, and an Mmc3 effector or a polynucleotide including a nucleotide sequence encoding an Mmc3 effector. The Mmc3 effector can by any Mmc3 effector, including but not limited to Mmc3 effectors that include the amino acid sequences of any of SEQ ID NOs:1-24, orthologs thereof, or variants thereof having at least 60% identity thereto. In various embodiments, the Mmc3 effector comprises an amino acid sequence selected from the group consisting of: BdMmc3 (SEQ ID NO:1); SfMmc3 (SEQ ID NO:2); SvMmc3 (SEQ ID NO:3); NapMmc3 (SEQ ID NO:4); ShMmc3 (SEQ ID NO:5); NoMmc3 (SEQ ID NO:6); PcMmc3 (SEQ ID NO:7); Sf2Mmc3 (SEQ ID NO:8); Sf3Mmc3 (SEQ ID NO:9); No2Mmc3 (SEQ ID NO:16); Sv2Mmc3 (SEQ ID NO:17); Rz2Mmc3 (SEQ ID NO:20); Rz3Mmc3 (SEQ ID NO:21); RzMmc3 (SEQ ID NO:22); Sf4Mmc3 (SEQ ID NO:23); Sv3Mmc3 (SEQ ID NO:24); Sf8Mmc3 (SEQ ID NO:25); and No3Mmc3 (SEQ ID NO:26); or comprises a variant of any thereof having at least 60% identity to any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:16, SEQ ID NO:17, SEQ ID NO:20, SEQ ID NO:21, SEQ ID NO:22, SEQ ID NO:23, SEQ ID NO:24, SEQ ID NO:25, or SEQ ID NO:26; or comprises a natually-occurring Mmc3 polypeptide having at least 50%, at least 55%, at least 60%, at least 65%, at least 70%, at least 75%, at least 80%, at least 85%, at least 90%, or at least 95% to any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:16, SEQ ID NO:17, SEQ ID NO:20, SEQ ID NO:21, SEQ ID NO:22, SEQ ID NO:23, SEQ ID NO:24, SEQ ID NO:25, or SEQ ID NO:26. Mmc3 effectors of the systems disclosed herein can have, for example, at least 60%, at least 65%, at least 70%, at least 75%, at least 80%, at least 85%, at least 90%, at least 95%, at least 96%, at least 97%, at least 98%, or at least 99% to SEQ ID NO:1, SEQ ID NO:2, SEQ ID NO:3, SEQ ID NO:5, SEQ ID NO:6, or SEQ ID NO:7. In further examples an Mmc3 effector of a CRISPR system as disclosed herein can have at least 60%, at least 65%, at least 70%, at least 75%, at least 80%, at least 85%, at least 90%, at least 95%, at least 96%, at least 97%, at least 98%, or at least 99% to SEQ ID NO:1, SEQ ID NO:2, SEQ ID NO:3, or SEQ ID NO:6. One of skill in the art can be guided by knowledge of conservative amino acid substitution, sequence alignments, crystal structures of effector proteins, and functional assays, such as but not limited to the plasmid interference assays detailed in the examples herein to assess the suitability of variants.

The Mmc3 effector can include a nuclear localization sequence (NLS) at the N-terminus, the C-terminus or both. In some embodiments, a vector encodes an Mmc3 effector comprising one or more nuclear localization sequences (NLSs), such as about or more than about 1, 2, 3, 4, 5, 6, 7, 8, 9, or 10 NLSs. In some embodiments, the RNA-modifying effector protein comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. The effector sequence and the NLS may in some embodiments be fused with a linker between 1 to about 20 amino acids in length.

Non-limiting examples of NLSs include an NLS sequence derived from: the NLS of the SV40 virus large T-antigen; the NLS from nucleoplasmin (e.g., the nucleoplasmin bipartite NLS); the c-myc NLS; the hRNPA1 M9 NLS; the IBB domain from importin-alpha; the NLS sequences of the myoma T protein, the p53 protein; the c-ab1 IV protein, or influenza virus NS1; the NLS of the Hepatitis virus delta antigen, the Mx1 protein; the poly(ADP-ribose) polymerase; and the steroid hormone receptors (human) glucocorticoid. In general, the one or more NLSs are of sufficient strength to drive accumulation of the RNA-modifying Mmc2 effector protein in a detectable amount in the nucleus of a eukaryotic cell.

The nucleotide sequence encoding the Mmc3 effector can optionally be codon optimized for a host of interest. (For reference, see Kim C. et al., Gene 1997, Vol 199, pages 293-301; Mauro V. et al., Trends Mol. Med., 2014, Vol. 20, pages 604-613.) Additional possible modifications include sequence modifications for improved function, such as but not limited to changing effector glycosylation sites.

In various embodiments, a polynucleotide that encodes an Mmc3 polypeptide includes a regulatory element, such as a promoter, operably linked to the sequence that encodes the Mmc3 polypeptide. Exemplary variations of the foregoing include a regulatory element selected from the group consisting of: CMV, RSV, SV40, EF1a, human beta actin, chicken beta actin, CAG, Ubc, TRE, UAS, Polyhedrin, CaMKIIa, GAL1, TEF, Ac5, GDS, ADH1, CaMV35S, Ubi, U6 and H1. The regulatory element is suitable for driving expression of an encoded polypeptide in a prokaryotic cell or a eukaryotic cell. Various embodiments of the latter contemplate a regulatory element suitable for driving expression of an encoded polypeptide in an animal cell, such as but not limited to a mammalian cell, or in a photosynthetic organism, such as a plant and algal cell.

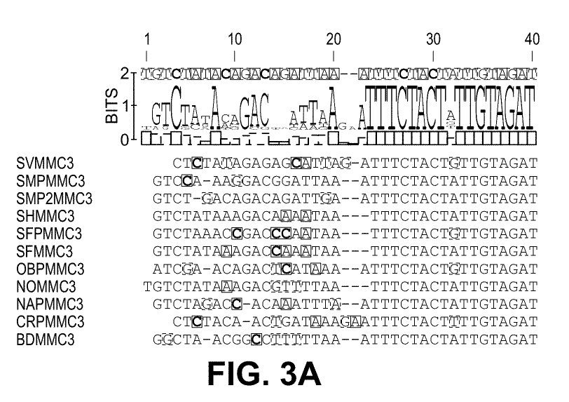

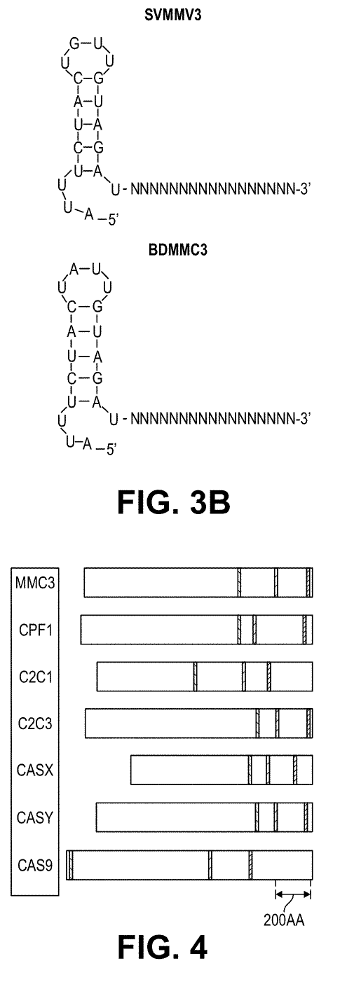

In certain embodiments, the engineered CRISPR system includes a guide RNA expression cassette. One or more engineered guide RNAs can be expressed and processed from an Mmc3 array or a portion thereof that includes a guide (targeting) sequence, typically an engineered or designed guide sequence, as a spacer sequence and is introduced into a target cell. In some embodiments, the expression vector includes a nucleotide sequence encoding a processed guide RNA sequence, such as a processed guide RNA sequence based on RNAseq analysis of a processed guide RNA structure, e.g., of E. coli engineered to include at least a portion of an Mmc3 array and effector. For example, one or more guide RNAs can be expressed from a construct that encodes a guide RNA that includes a guide (targeting) sequence fused to at least a portion of a repeat sequence of an Mmc3 array or a sequence derived from a repeat sequence of an Mmc3 array (see FIG. 3A). The guide sequence having homology to the target sequence can be, for example between seventeen and twenty-seven nucleotides in length, for example, between eighteen and twenty-five nucleotides in length or between about eighteen and about twenty-three nucleotides in length. The CRISPR repeat sequence included in the guide RNA guide or construct can be between about 16 and about 30 nucleotides in length, such as between about seventeen and about twenty-five nucleotides in length, or between about eighteen and about tweny-three nucleotides in length and can be fused to the 5′ end or 3′ end of the guide sequence. In various examples the sequence derived from an Mmc3 repeat sequence is fused to the 5′ end of the spacer, or targeting sequence. The guide RNA (guide RNA) encoding sequence can be operably linked to a promoter operable in the target cell such as a U6 promoter.

An exemplary engineered Class 2 CRISPR system includes e.g, a guide RNA or nucleic acid molecule for expressing a guide RNA, where the guide RNA is designed to target a nucleic acid molecule of interest, and an Mmc3 effector polypeptide or a nucleic acid molecule encoding an Mmc3 effector polypeptide, where the guide RNA or nucleic acid encoding the guide RNA and Mmc3 effector or nucleic acid molecule encoding the Mmc3 effector may be introduced into a target cell or tissue simultaneously or sequentially. The guide RNA (or guide RNA or CRISPR array) and effector module may be introduced into the cell as polynucleotides, e.g., DNA or RNA or both, although administration of polypeptide forms of the effector are also useful, as well as combinations of polypeptides and polynucleotides (such as a nucleic acid guide sequence and a nuclease enzyme, which may be complexed prior to delivery).

Mmc3 CRISPR systems can also function in vitro. For example, an Mmc3 effector gene can be cloned into an expression vector for a specific host system such as E. coli. A range of E. coli hosts and vectors designed for protein expression are known to the art (e.g. pET systems, pMAL systems, pBAD systems). An epitope tag may be included as a translational fusion to the N or C-terminus, or both. Examples of tags include His, Strep, and Maltose Binding Protein (MBP), as nonlimiting examples. Purification of the Mmc3 effector can be performed using methods known in the art, for example, using the manufacturer's instructions if a commercially available expression vector is used, and may depend on the specific expression and epitope combination utilized. To perform an in vitro nuclease assay, purified Mmc3 effector and an in vitro transcribed guide RNA (guide RNA) compatible with the Mmc3 effector can be combined in a suitable buffer. The guide RNA is designed to include a spacer sequence that hybridizes to the desired target sequence. After combining the Mmc3 and guide RNA, target DNA which can be, for example, a PCR product, plasmid DNA, or genomic DNA or a fragment of any thereof, is added to the reaction mixture. After a period of incubation at the optimal temperature for the enzyme, the target DNA is recovered and analyzed for cleavage at the targeted site.

In one aspect, the engineered Mmc3 CRISPR system is for modifying a nucleic acid molecule in a plant cell. The methods include introducing Mmc3 CRISPR system as described herein to target one or more plant genes to confer desired traits on essentially any plant. A wide variety of plants and plant cell systems may be engineered to include a non-naturally occurring Mmc3 CRISPR system using the nucleic acid constructs and various transformation methods known in the art (See Guerineau F., Methods Mol Biol. (1995) 49:1-32). In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot potato, sugar beets, yam), leafy vegetable crops (e.g., lettuce, spinach); flowering plants (e.g., petunia, rose, chrysanthemum), conifers and pine trees (e.g., pine fir, spruce), plants used in phytoremediation (e.g., heavy metal accumulating plants); oil crops (e.g., sunflower, rape seed) and plants used for experimental purposes (e.g., Arabidopsis). Thus, the methods and Mmc3 CRISPR systems can be used over a broad range of plants, such as for example with dicotyledonous plants belonging to the orders Magniolales, Miciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods and Mmc3 CRISPR systems can also be used with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g those belonging to the orders Pinales, Ginkgoales, Cycadales, Araucariales, Cupressales and Gnetales.

An Mmc3 system can also be used to modify genomes of microorganisms, including fungii, Labyrinthulomycetes, and algae. In some embodiments, the microorganism used for genome modification is a photosynthetic microorganism. In some embodiments, the photosynthetic microorganism is a eukaryotic microalga. In some embodiments, the eukaryotic microalga is a species of Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Picochlorum, Platymonas, Pleurochrysis, Pleurococcus, Prototheca, Pseudochlorella, Pseudoneochloris, Pyramimonas, Pyrobotrys, Scenedesmus, Schizochlamydella, Skeletonema, Spyrogyra, Stichococcus, Tetrachorella, Tetraselmis, Thalassiosira, Viridiella, or Volvox.

### Vectors

For in vivo editing, polynucleotides encoding the guide RNA and/or the effector are commonly incorporated into vectors for introduction into target cells. “Vector” as used herein refers to a recombinant DNA or RNA plasmid or virus that comprises a heterologous polynucleotide capable of being delivered to a target cell, either in vitro, in vivo or ex-vivo. The heterologous polynucleotide can comprise a sequence of interest and can be operably linked to another nucleic acid sequence such as promoter or enhancer and may control the transcription of the nucleic acid sequence of interest. As used herein, a vector need not be capable of replication in the ultimate target cell or subject. The term vector may include expression vector and cloning vector.

Vectors include, but are not limited to, nucleic acid molecules that are single-stranded, double-stranded, or partially double-stranded; nucleic acid molecules that comprise one or more free ends, no free ends (e.g., circular, relaxed or supercoiled); nucleic acid molecules that comprise DNA, RNA, or both; and other varieties of polynucleotides known in the art. An exemplary vector is a plasmid, into which additional DNA segments can be inserted, such as by standard molecular cloning techniques. Another type of useful vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a target cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a target cell upon introduction into the cell, and thereby are replicated along with the host genome. Other genomes appropriate to target include e.g., chloroplast, mitochondrial, plastid, bacteriophage and viral genomes. Certain vectors are capable of directing the expression of genes to which they are operatively-linked. Such vectors are commonly referred to as expression vectors. It will be appreciated by those skilled in the art that the selection and design of the vector will depend on such factors as the choice of target cell to be transformed, the level of expression desired, whether constitutive or conditional expression is desired, whether stable or transient expression is desired, and other such factors that shall be apparent to a skilled artisan. Accordingly, the invention includes one or more of the Mmc3 family effector nucleases in a vector. Many vectors are suitable for cloning and expressing an Mmc3 family effector. A currently preferred vector will express an Mmc3 family polypeptide in a eukaryotic cell. Most preferred would be a vector suitable for expression of the nuclease in a mammalian, and even a human cell. Such vectors commonly include one or more regulatory elements, which can be constitutive or inducible, and would drive expression of an Mmc3 family polypeptide. Vectors designed for tissue specific expression are widely used, and within the scope of this invention.

Vectors can be designed for expression of CRISPR components (e.g. nucleic acid transcripts, proteins, or enzymes) in prokaryotic or eukaryotic cells. For example, CRISPR transcripts can be expressed in prokaryotic cells, for example bacterial cells such as Escherichia coli, or eukaryotic cells, such as yeast cells, insect cells (using baculovirus expression vectors), or mammalian cells. Typically, such an expression system will have a vector with a first regulatory element operably linked to a CRISPR RNA nucleotide sequence, and will express a guide RNA; and a second regulatory element operably linked to a polynucleotide sequence encoding a Mmc3 effector. Alternatively, one vector may encode the guide RNA, and another vector may encode an Mmc3 protein. A single vector encoding bicistronic elements encoding the guide RNA and the Mmc3 polypeptide is currently preferred. The vector system may include the full expression cassettes for a CRISPR/Mmc3 systems, that when expressed in the cell (prokaryotic or eukaryotic), provides for a single guide sequence that can hybridize to a target sequence that is 3′ to a Protospacer Adjacent Motif (PAM), and the guide RNA can form a complex with the Mmc3 effector polypeptide.

Promoter, enhancers and associated 5′- and 3′-regulatory elements useful for vectors are exemplified in Goeddel, GENE EXPRESSION TECHNOLOGY: METHODS IN ENZYMOLOGY 185, Academic Press, San Diego, Calif. (1990). The recombinant expression vector can be transcribed and translated in vitro, for example using T7 promoter regulatory sequences and T7 polymerase. Vectors may be introduced and propagated in a prokaryote to amplify copies of a vector to be introduced into a eukaryotic cell or as an intermediate vector in the production of a vector to be introduced into a eukaryotic cell. For example, bacterial expression systems involve a vector such a pBluescript, a pET, a pBAD, comprising a promoter and associated regulatory elements, transformation-competent bacteria, and transformation media, transformation methods and tools such as heat shock method or electroporator, and bacterial culture and growth media. (For reference, see Molecular Cloning: A Laboratory Manual, Vol. 1, Chapter 3, J. F. Sambrook and D. W. Russell, ed., Cold Spring Harbor Laboratory Press).

For eukaryotic recombinant expression vector systems, the vector choices span Adenoviral vectors, Adeno-associated virus (AAV) vectors, retroviruses vectors, Lentiviruses, MMLV, Piggybac viral vectors, and several other bacterial vectors. Vectors are readily available, where most vectors may be adapted towards an inducible expression system such as Tetracycline on-off vector systems, or towards constitutive expression systems, where the system may be designed for strong (high copy number) expression, such as using Cytomegalovirus (CMV) promoter, or mild to moderate copy number expression such as U6 or Ptet promoter, are well-known in the art. For reference, please see: An Introduction to Genetic Analysis. 7th edition. Griffiths A J F, Miller J H, Suzuki D T, et al. New York: W. H. Freeman; 2000; or, Molecular Cell Biology. 4th edition. Lodish H, Berk A, Zipursky S L, et al., New York: W. H. Freeman; 2000. In vivo and tissue-specific timed expressions may be achieved by using Cre-Lox systems (Reviewed in Duyne G., Annu Rev BioPhys Biomol Struct., 2001, 30: 87-104). For test purposes, the vectors may comprise a tag, such as green fluorescence protein, Renella luciferase system, a small molecule tag such as HA-tag or FLAG-tag, to assist in detection of expression. Alternatively, expression of the DNA in the recombinant vector expression cassette can be readily determined by routine methods such as quantitative reverse transcriptase PCR, sequencing or protein expression analyses.

Transfection of expression vectors in various cell types is available to one of skill in the art as a routine laboratory methodology. Additionally, predesigned and custom vector systems and protocols are available from various manufacturers. Optimized protocols for molecular cloning, transfection and expression procedures will be apparent to a skilled artisan in view of the teachings herein. Accordingly, the invention provides a cell having a transfected Mmc3 family nuclease, and/or an expression cassette having an Mmc3 family nuclease, that may further include additional CRISPR system elements. Currently preferred are mammalian and human cells having such vector systems.

Recombinant expression vectors comprise polynucleotides for expressing a Mmc3 polypeptide. The polynucleotide encoding an Mmc3 enzyme is operably linked to regulatory elements. Regulatory elements include 5′ and 3′ regulatory elements. The 5′ regulatory elements include a promoter, and optionally, an enhancer, and generally, all elements upstream to the gene which help in the control of the expression of the gene that is to be transcribed. In cases where expression of another protein is needed to control the promoter, such as in a Tet-on-off system, where expression of tetracycline is necessary for the promoter to become active or to become dormant as the system is designed, a separate vector may express the trans-element, such as tetracycline. The 5′-regulatory elements are generally constructed upstream of the first amino acid, methionine, encoded by the trinucleotide: AUG. In some cases the promoter may be directly upstream of the recombinant gene, in other cases it may be spaced by a few nucleotide bases. Predesigned vectors offer optimized expression systems where the recombinant gene and the vector are both digested with same restriction enzymes, or with enzymes that cleave at the same sites, (isoschizomers), the generate the cloning ends and ligate the complementary sticky nucleotide ends (generated by the restriction digest) of the vector and the insert (in this case the Mmc3 encoding gene), and ligate, thereby generating the expression vector comprising an Mmc3 expression cassette. The 3′-regulatory elements are usually stretches of polynucleotides which help in the processing of the RNA and the overall stability of the transcript. In general, 3′ untranslated regions (3′-UTRs) may be part of the insert where the 3′ segment of the gene of interest is present in the portion of polynucleotide to be clones into the vector, or a 3′-UTR element may be present universally as a part of the vector, in which case it is a heterologous 3′-UTR but serving the same essential functions.

As described above, the vector may contain a regulatory element. A “regulatory element” as used herein includes promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. Such regulatory element would be operably configured to express e.g., an Mmc3 effector polypeptide, or nucleic acid component(s). For example and without limitation, expression of an Mmc3 effector polypeptide in a human cells is accomplished using CMV expression vectors. In such example, a U6 promoter is fused to a guide RNA sequence. Other common regulatory elements useful in vectors having the adaptor module, CRISPR array or effector module include: pol I, II or pol III promoters such as U6 and H1, RSV LTR promoter and/or enhancer), CaMV promoter/enhancer, CMV promoter/enhancer, SV40 promoter, β-actin promoter, DHFR promoter, PGK promoter, and the EF1α promoter, R-U5′ segment in LTR of HTLV-I, SV40 enhancer, human beta actin, chicken beta actin, CAG, Ubc, TRE, UAS, Polyhedrin, CaMKIIa, GAL1, TEF, Ac5, GDS, ADH1, CaMV35S, Ubi, and others generally known to those of skill in the art.

A vector is introduced into a target cell or tissue. The method will depend on the particular vector used and the target cell. As described above, the adaptor module, guide RNA or guide RNA and the effector module may be introduced via nucleic acid, either DNA or RNA; either by plasmid or viral vectors. The guide RNA may be processed or unprocessed. The various components of the system may further be delivered alone or in combination, or the effector may be provided as a protein rather than a nucleic acid. For example, nucleic acid components comprising the guide RNA or guide RNA may be preassembled with an effector protein then introduced into the target cell. Alternatively, the various components of the system may further be delivered sequentially, for example an effector may be introduced into the target cell, either as a protein or as a nucleic acid sequence encoding the effector protein, that is, introduced in a fashion that is stable in host cell, e.g., permanently (under selective pressure), or extra-chromosomal element, or within the host genome, where it can be regulated/induced, or transiently, and the nucleic acid comprising the guide RNA may be introduced separately. By way of further illustration, one or more of the various components may be introduced to the cell, and expression of these may be induced in a selective manner One of skill in the art could determine alternative variations to achieve the ultimate combination of the system elements to form a functional complex.

Vectors for delivery of polynucleotides are commonly formulated into a delivery system, for example the vectors are delivered via particles, vesicles, or viral vectors. For example, exosomes or liposomes are common delivery vehicles for cellular transfection, and viral vectors such as adenovirus, lentivirus or adeno-associated virus (AAV) are capable of active infection of target cells. Electroporation provides another common transfection method. Sequencing can confirm successful transfection of target cells. Target cells that are useful for the present invention include plant cells, prokaryotic cells, and eukaryotic cells. Prokaryotic cells are exemplified by bacteria e.g., cyanobacteria and archaea; eukaryotic cells are exemplified by e.g., animal cells, plant cells, algae; unicellular or multicellular organisms and fungal cells. Currently preferred are animal cells such as mammalian cells and more particularly human cells or tissues, for example but not limited to somatic cells and stem cells or stem cell lines. Sequencing can confirm the presence of the transgene. Nuclease assays can confirm enzyme expression from the transgene and confirm function.

The guide RNA or guide RNA can be generated from the CRISPR array, which is composed of direct repeats flanking unique spacer sequences. After processing, individual spacer-repeat guide RNA sequences are complexed with an effector Cas protein such as one of the Mmc3 family nucleases of the present invention. Hybridization of the spacer with the complimentary protospacer target sequence directs the Mmc3 nuclease to cleave the target nucleic acid at a predetermined and specific location. As an additional layer of specificity, cleavage typically requires a Protospacer Adjacent Motif (PAM) either 5′ or 3′ of the protospacer sequence. In the Mmc3 systems disclosed herein, the PAM is 5′ of the target sequence.

In various embodiments, the Mmc3 effectors referred to herein encompass a homologue or an orthologue of an Mmc3 protein as disclosed herein. The terms “ortholog” and “homolog” are well known in the art. By means of further guidance, a homolog of a gene is related to the reference gene by descent from a common ancestral gene and the homologs typically are structurally similar, e.g., sequence homology; and an ortholog of a gene refers to a homologous gene derived from a common ancestral gene in which the genes have approximately similar function across species Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. doi: 10.1002/pro.2225.). See also Shmakov et al. (2015) for application in the field of CRISPR-Cas loci. In particular embodiments, the homolog or ortholog of Mmc3 as referred to herein has a sequence homology or identity of at least 60%, more preferably at least 65%, even more preferably at least 70%, such as for instance at least 75% with an Mmc3 effector such as any disclosed herein. In further embodiments, the homolog or ortholog of an Mmc3 effector as disclosed herein has a sequence identity of at least 80%, at least 85%, at least 90%, or at least 95% with an Mmc3 effector as disclosed herein. Orthologs of Mmc3 may be found in organisms which include but is not limited to species of the genera Smithella, Candidatus, Sulfuricurvum Omnitrophica, and Porphyromonas, as nonlimiting examples.

Further considered for use in the systems and methods provided herein are Mmc3 effector variants, where a variant has one or more mutations and has a sequence identity of at least 60%, at least 65%, at least 70%, or at least 75% at least 80%, at least 85%, at least 90%, or at least 95% with an Mmc3 effector such as any disclosed herein.

### Methods of DNA Modification

CRISPR systems are useful for modifying gene sequences. Provided herein are methods of modifying a target nucleic acid molecule in vivo, where the method includes delivering to a cell comprising one or more nucleic acid molecules comprising one or more target nucleic acid sequences a non-naturally occurring or engineered composition that includes:
a) one or more polynucleotide sequences comprising one or more guide RNAs, or one or more polynucleotide sequences encoding one or more guide RNAs, wherein the one or more guide RNAs is designed to form a complex with the Mmc3 effector and designed to hybridize with a target nucleic acid sequences, and
b) an Mmc3 effector protein, or a nucleotide sequence encoding an Mmc3 effector protein; where the one or more guide RNAs form one or more complexes with the Mmc3 effector protein, resulting in cleavage of the one or targeted nucleic acid molecules thereby modifying the one or more target nucleic acid molecules. The percentage of target nucleic acid molecule modification using the method can be at least 5%. Where the modification is target nucleic acid cleavage, in vivo modification can be assessed, for example, by plamid interference assays as demonstrated herein. Where the cell into which the system components are delivered has active DNA repair mechanisms, DNA modification can include mutation of the DNA sequence at the target site, for example, by insertion or deletion of nucleotides. Mutations can be assessed by assays that include, for example, surveyor assays, DNA sequencing, PCR, gel electrophoresis, and/or phenotypic assays. In some examples, the system delivered to the target cells further includes a donor or repair fragment that allows a phenotypic assay for target site modification as illustrated for example in Example 6 herein.

In some embodiments, the percentage of modified target nucleic acid molecules can be assessed as the percentage of cleaved nucleic acid molecules, where the percentage of cleaved nucleic acid molecules using the methods is at least 5%. In various embodiments, the percentage of target nucleic acid molecule modification is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Target nucleic acid molecule cleavage can be assessed, for example, using assays such as plasmid depletion or interference assays, where the percentage calculated takes into account the background occurring in cells that include CRISPR-Cas systems that are identical except that they include an incorrect guide (spacer) sequence or PAM.

Accordingly, in various aspects the invention provides a gene editing method wherein, the method provides for delivering an engineered or non-naturally occurring Mmc3 CRISPR system to a cell containing a target gene including a target sequence, thereby cleaving the target sequence and thereby editing the target nucleic acid molecule or gene. In various embodiments of such methods, the effector and the guide RNA are delivered to the cell as polynucleotides. In the above methods, various additional embodiments provide for delivery of the vector to the cell via electroporation, transfection, conjugation, particle bombardment, lipofection, nucleofection, calcium phosphate precipitation, liposomes, peptide-mediated transformation, particles, or vesicles. In some embodiments, the vector is viral, and delivery of the polynucleotide is accomplished by infection of the cell. Exemplary viral vectors include adenovirus, lentivirus and adeno-associated virus (AAV).

In other embodiments, the effector is delivered to the cell as a polypeptide and the guide RNA is delivered to the cell as a polynucleotide. In some embodiments, the effector and guide RNA are complexed prior to cellular delivery. Delivery of proteins or nucleoprotein complexes can be via electroportaion, peptide-mediated delivery, particle bombardment, liposomes, or other methods.

In some embodiments of the method, an Mmc3 effector gene can be introduced into a cell and the cell expressing the Mmc3 effector gene can subsequently be transformed with at least one guide RNA molecule targeting a nucleic acid molecule in the cell, resulting in modification of the targeted nucleic acid molecule.

In various embodiments, the frequency of target nucleic acid modification can be at least 5%, at least 10%, at least 15%, at least 20%, at least 35%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98%. Target nucleic acid modification can be nucleic acid cleavage or mutation, where mutation at the site of cleavage by the effector polypeptide complex occurs via cellular DNA repair mechanisms. The target nucleic acid molecule can be episomal DNA or genomic DNA. The host cell can be a eukaryotic or prokaryotic host cell.

In some embodiments, the target nucleic acid molecule is further modified by the integration of a polynucleotide, e.g., a donor or repair nucleic acid molecule, into the cleaved target sequence. The donor molecule can optionally include a sequence encoding a selectable marker. The donor molecule can optionally include sequences on one or both ends that have homology to a genetic locus of interest to facilitate introduction of the donor DNA into the locus by homologous recombination.

The above methods provide for use of a RuvC domain containing effector polypeptide in a CRISPR/Cas system within a cell for altering gene expression in the cell. Specifically, but without limitation, the invention contemplates use of an Mmc3 effector polypeptide in a eukaryotic cell for altering a target gene sequence in the cell.

Further provided are CRISPR-Cas systems that include Mmc3 ORF3 polypeptides for increasing the efficiency of genome editing by effectors such as Mmc3 and Cpf1 effectors. In exemplary embodiments the Mmc3 ORF3 polypeptides have at least 30%, at least 35%, at least 40%, at least 45%, at least 50%, at least 55%, at least 60%, at least 65%, at least 70%, at least 75%, at least 80%, at least 85%, at least 90% or at least 95% identity to an amino acid sequence selected from the group consisting of SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58.

### EXAMPLES

### Example 1

### Identifying and Isolating Mmc3 Family Effectors

Mmc3 effector proteins were identified using bioinformatics analysis of genomes of species of Smithella, Sulfuricurvum, Porphyromonas, and Candidatus genera, as well as bacterial species of unknown genera. Various strategies were used including identification of contigs containing Cas1 and CRISPR array flanked by a large (>800 amino acid) protein of unknown function and identification of evolutionarily distant CRISPRs using Hidden Markov Models (HMM) of relevant effector types. Novel effectors were subsequently used as queries to perform BLAST searches of NCBI databases. Based on these methods, twenty-six Mmc3 systems were identified: eighteen complete systems encoding an effector and an associated CR array (e.g, FIG. 1), as well as eight partial or incomplete systems, of which three had complete Mmc3 effector genes identified (see Table 2).

To identify a new family of effectors, a Cas1 HMM was trained on a combination of proprietary and public protein sequence data (about 75.8 million proteins). HMMER v.3.1b2 was used to iteratively search the dataset for Cas1, updating the HMM each time. This process recapitulates the steps taken by jackhmmer (ebi.ac.uk/Tools/hmmer/search/jackhmmer). The loop runs five times or until the model converges, whichever comes first. The output of this search contained contigs very likely to contain Cas1. These contigs were run through PILER-CR v.1.06 to identify the subset likely to have CRISPR repeat sequences. Cas1 hits for which a known CRISPR effector (Cas3, Cas9, Cmr4, or Csm3) was detected within five genes were discarded. The results were searched for Cas1 genes in which the largest upstream gene was at least 800 amino acids in length. Mmc3 effectors NoMmc3 (SEQ ID NO:6) and SfMmc3 (SEQ ID NO:2) were discovered among these results. Subsequent Blastp analysis using the NoMmc3 (SEQ ID NO:6) and SfMmc3 (SEQ ID NO:2) effector sequences as queries against SGI-proprietary and public sequence databases recovered genes encoding BdMmc3 (SEQ ID NO:1), SvMmc3 (SEQ ID NO:3), NapMmc3 (SEQ ID NO:4), ShMmc3 (SEQ ID NO:5), PcMmc3 (SEQ ID NO:7), Sf2Mmc3 (SEQ ID NO:8), and Sf3Mmc3 (SEQ ID NO:9).

Discovered effector genes BdMmc3 (SEQ ID NO:221), SfMmc3 (SEQ ID NO:222), SvMmc3 (SEQ ID NO:223), NapMmc3 (SEQ ID NO:224), ShMmc3 (SEQ ID NO:225), and NoMmc3 (SEQ ID NO:226) were discovered in the context of complete Mmc3 effector systems that included an open reading frame encoding the effector protein and a CRISPR array (FIG. 1). An open reading frame unrelated to any previously identified polypeptide-encoding sequences in CRISPR loci and referred to herein as ORF3 was found in the CRISPR loci of multiple Mmc3 systems. ORF3 sequences are aligned in FIG. 2(A-C).

Also discovered were genes encoding a complete Mmc3 effector where the sequence of the Mmc3 CRISPR system was incomplete, including the PcMmc3 effector system that included a gene (SEQ ID NO:227) encoding a complete PcMmc3 effector (SEQ ID NO:7), the RzMmc3 effector system that included a gene (SEQ ID NO:242) encoding a complete RzMmc3 effector (SEQ ID NO:22), and the Sf8Mmc3 effector system that included a gene (SEQ ID NO:S) encoding a complete Sf8Mmc3 effector (SEQ ID NO:25). CRISPR systems where the uncovered effector gene was incomplete, providing only a partial protein sequence, include Sf2Mmc3 (partial gene sequence SEQ ID NO:228 encoding SEQ ID NO:8), Sf3Mmc3 (partial gene sequence SEQ ID NO:229 encoding SEQ ID NO:9), Bd2Mmc3 (partial gene sequence SEQ ID NO:238 encoding SEQ ID NO:18), Bd3Mmc3 (partial gene sequence SEQ ID NO:239 encoding SEQ ID NO:19), and Rz3Mmc3 (partial gene sequence SEQ ID NO:241 encoding SEQ ID NO:21). See, Table 2.

At least six additional Mmc3 effectors were identified by querying additional databases: Smp3Mmc3 (WP_039658699, SEQ ID NO:10); SmpMmc3 (KFO67988.1, SEQ ID NO:11); Smp2Mmc3 (MAEO01000208, SEQ ID NO:12); CrpMmc3 (LBTJ01000016, SEQ ID NO:13); ObpMmc3 (MHGE01000059, SEQ ID NO:14); and SfpMmc3 (WP_041148111, SEQ ID NO:15). See, Table 3.

Further, additional proprietary metagenomics sequence data was searched for Mmc3 effectors using a Hidden Markov model derived from multiple protein sequence alignments of the identified Mmc3 effectors. HMMER v.3.1b2 was again used, this time to iteratively search sequence data for new Mmc3 effectors, updating the HMM each time. This behavior recapitulates the steps taken by jackhmmer (ebi.ac.uk/Tools/hmmer/search/jackhmmer). The loop runs five times or until the model converges, whichever comes first. The output of this search contained contigs very likely to contain Mmc3 effectors or related proteins. Contigs were manually curated for the presence of Mmc3, CRISPR arrays, and accessory genes (e.g. cas1, cas2, ORF3). From this analysis, two additional complete Mmc3 systems (Sv2Mmc3, No2Mmc3) were discovered that minimally consist of a full length Mmc3-encoding sequence and CRISPR repeats (FIG. 1 and Table 2). The No2Mmc3 effector (SEQ ID NO:16) is approximately 57% identical to the NoMmc3 effector (SEQ ID NO:6), whereas the Sv2Mmc3 effector (SEQ ID NO:17) is approximately 94% identical to the SvMmc3 effector (SEQ ID NO:3). In addition to the No2Mmc3 and Sv2Mmc3 full length Mmc3 systems, a number of partial Mmc3 contigs were identified (Table 2; SEQ ID NOs:18, 19, 21, and 25 provide amino acid sequences encoded by partial effector genes) as well as additional systems where the complete Mmc3 effector gene was identified (encoding the Rz2Mmc3 (SEQ ID NO:20), RzMmc3 (SEQ ID NO:22), Sf4Mmc3 (SEQ ID NO:23), Sv3Mmc3 (SEQ ID NO:24), Sf8Mmc3 (SEQ ID NO:25), and No3Mmc3 (SEQ ID NO:26) effectors).

CRISPR arrays were detected using the bioinformatics software CRISPRdetect and CRISPRfinder: (brownlabtools.otago.ac.nz/CRISPRDetect/predict_crispr_array.html) and (crispr.i2bc.paris-saclay.fr/Server/). Each full length Mmc3 system has a CRISPR array proximal to the Mmc3 effector protein. Direction of Mmc3 CRISPR arrays was assessed bioinformatically using CRISPRdetect, CRISPRmap (rna.informatik.uni-freiburg.de/CRISPRmap/Input.jsp) and by direct analysis of the repeat secondary structure predictions. Alignment of CRISPR repeats shows a highly conserved 3′ region that is predicted to form a hairpin structure (see, FIG. 3A and FIG. 3B). The consensus sequence at the 3′ end of the repeat, ATTTCTACTDTTGTAGT (SEQ ID NO:44) is similar to that described in the Cpf1 CRISPR system (Zetsche et al. (2015) Cell, 163: 759-771), where the processed repeat of Mmc3 systems may differ from that of a Cpf1 system by only a single nucleotide.

TABLE 2
Mmc3 Systems

| | | | cds | | |
|---|---|---|---|---|---|
| | Polypeptide | Length | SEQ ID | | |
| Effector | SEQ ID NO | (aa) | NO | Description | Taxonomic Grouping |
| BdMmc3 | 1 | 1241 | 221 | Complete Mmc3 system | Bacteroidales |
| SfMmc3 | 2 | 1298 | 222 | Complete Mmc3 system | Sulfuricurvum sp. |
| SvMmc3 | 3 | 1285 | 223 | Complete Mmc3 system | Sulfuricurvum sp. |
| NapMmc3 | 4 | 1033 | 224 | Complete Mmc3 system | Bacteria |
| ShMmc3 | 5 | 1187 | 225 | Complete Mmc3 system | Smithella |
| NoMmc3 | 6 | 1172 | 226 | Complete Mmc3 system | Bacteria |
| PcMmc3 | 7 | 1270 | 227 | Full Mmc3 Effector, | Porphyromonas |
| | | | | incomplete system | |
| Sf2Mmc3 | 8 | 1040 | 228 | Partial Mmc3 Effector | Sulfuricurvum sp. |
| Sf3Mmc3 | 9 | 1042 | 229 | Partial Mmc3 Effector | Sulfuricurvum sp. |
| No2Mmc3 | 16 | 1177 | 236 | Complete Mmc3 system | Smithella sp. |
| Sv2Mmc3 | 17 | 1290 | 237 | Complete Mmc3 system | Sulfuricurvum sp. |
| Bd2Mmc3 | 18 | 447 | 238 | Partial Mmc3 Effector | No assignment |
| Bd3Mmc3 | 19 | 419 | 239 | Partial Mmc3 Effector | No assignment |
| Rz2Mmc3 | 20 | 1030 | 240 | Complete Mmc3 system | Candidatus |
| | | | | | Roizmanbacteria |
| Rz3Mmc3 | 21 | 470 | 241 | Partial Mmc3 Effector | Candidatus |
| | | | | | Roizmanbacteria |
| RzMmc3 | 22 | 1030 | 242 | Full Mmc3 Effector, | Candidatus |
| | | | | incomplete system | Roizmanbacteria |
| Sf4Mmc3 | 23 | 1258 | 243 | Complete Mmc3 system | Sulfuricurvum sp. |
| Sv3Mmc3 | 24 | 1297 | 244 | Complete Mmc3 system | Sulfuricurvum sp. |
| Sf8Mmc3 | 25 | 1291 | 245 | Full Mmc3 Effector, | Sulfuricurvum sp. |
| | | | | incomplete system | |
| No3Mmc3 | 26 | 1180 | 246 | Complete Mmc3 | No assignment |
| | | | | system | |

TABLE 3
Additional Mmc3 systems

| | Polypeptide | | | cds | | |
|---|---|---|---|---|---|---|
| | SEQ ID | Length | Protein | SEQ ID | | Source |
| Effector | NO | (aa) | Accession | NO | Gene region | Information |
| Smp3Mmc3 | 10 | 1084 | KIE18642 | 230 | JMED01000011 | Smithella sp. |
| | | | | | | SC_KO8D17 |
| SmpMmc3 | 11 | 1064 | KFO67988 | 231 | JQDQ01000121 | Smithella sp. |
| | | | | | | SCADC |
| Smp2Mmc3 | 12 | 1217 | none | 232 | MAEO01000208 | Smithella sp. M82 |
| CrpMmc3 | 13 | 1057 | KKQ38176 | 233 | LBTJ01000016 | Candidatus |
| | | | | | | Roizmanbacteria |
| | | | | | | bacterium |
| | | | | | | GW2011_GWA2_37_7 |
| ObpMmc3 | 14 | 1067 | OGX23684 | 234 | MHGE01000059 | Omnitrophica |
| | | | | | | WOR_2 bacterium |
| | | | | | | GWF2_38_59 |
| SfpMmc3 | 15 | 1232 | KIM12007 | 235 | JQIT01000003 | Sulfuricurvum sp. |
| | | | | | | PC08-66 |

Mmc3 systems were assessed for the presence of tracrRNA by analyzing intergenic regions for sequence with partial complementarity to CRISPR repeat sequences. Suboptimal alignments failed to uncover strong evidence for an anti-repeat sequence typical of tracrRNA from other systems (i.e. Cas9, C2c1). Based on this analysis, Mmc3 systems were considered unlikely to require accessory RNAs for their activity, e.g., a tracr RNA, as confirmed by subsequent experiments.

Mmc3 systems were found to be represented by a diverse set of system architectures (see, for example, FIG. 1). The minimal system included an Effector (Mmc3) and a CRISPR (Cr) array, where the CRISPR array includes two or more CRISPR repeats separated by unique spacer sequences. Several of the identified systems (e.g., NoMmc3 and SfMmc3) encode Cas4, Cas1 and/or Cas2 genes, however not all Mmc3 systems do, reinforcing that these Cas genes are not required for nuclease activity across the Mmc3 family. Nine of the identified systems were found to encode a conserved protein, referred to herein as ORF3, that has not been described in other CRISPR systems.

Overall sequence homology between Mmc3 effector proteins and effector proteins of other CRISPR systems is low, with Cpf1 effector proteins having the highest sequence identity to Mmc3 effectors at 8-12%. Sequence identity this low likely suggests differences in overall protein folding (Rost, 1999).

Multiple sequence alignments of Mmc3 with other Class 2 CRISPR Effectors (Cpf1, C2c1, C2c3, CasX, CasY, and Cas9) were of low quality but allowed for identification of the RuvC I and RuvC III catalytic motifs (defined in Aravind et al. (2000) Nucl Acids Res., 28: 3417-3432). The RuvC I and RuvC III regions of Mmc3 possess the known catalytic residues but show pronounced variation in surrounding residues known to play key roles in nuclease function. By contrast, whole-protein alignments were not sufficient to allow identification of the RuvC II domain of Mmc3, which is a strong predictor of DNA cleavage activity (Zetsche et al. (2015), ibid). Examination of crystal structures for Class II effector proteins (i.e., SpCas9, LbCpf1, AsCpf1, etc.) reveals a hydrophobic pocket around the active site formed by residues neighboring each of the three RuvC catalytic residues. The amino acid identity at these positions is not conserved but is limited to those with hydrophobic side chains. Based on this analysis, the RuvC II motif is more accurately defined by 3-4 hydrophobic residues directly before the catalytic glutamate and one hydrophobic residue two positions after the catalytic glutamate. The small size of the sequence motif and its limited conservation (hydrophobic residues, not specific amino acids) make identification of the RuvC II motif in Mmc3 difficult, but searching a multiple sequence alignment of more than ten Mmc3 sequences allowed for identification of RuvC II. Discovery of sufficient representatives of the Mmc3 sub-type was critical to generation of an accurate alignment and identification of the RuvC II domain. The location of the RuvC II motif within the Mmc3 protein is substantially different relative to the other Class II CRISPR effectors and partly explains the difficulty in identifying it using primary sequence alignments.

Like many other Class 2 CRISPR Effectors, the three active site motifs of the RuvC domain of Mmc3 are non-contiguously spread over the protein sequence. Similar to the Type V effectors Cpf1, C2c1, C2c3, CasX, and CasY, the three RuvC catalytic motifs of Mmc3 are all contained in the C-terminal region, whereas the RuvC of Cas9 is spread across the entire effector polypeptide sequence (shown schematically in FIG. 4). The spacing between RuvC catalytic motifs is different in each effector sub-type, with that of Mmc3 most closely resembling that of CasY. However, there is substantially more amino acid sequence between RuvC I and II in Mmc3 than in CasY (approximately 200 amino acids in Mmc3 and approximately 70 amino acids in CasY). Overall, the spacing and position of RuvC domains is different for all Type V sub-types, including Mmc3 (see Table 4). For example, the Mmc3 effectors disclosed herein, including BdMmc3 (SEQ ID NO:1), SfMmc3 (SEQ ID NO:2); SvMmc3 (SEQ ID NO:3); NapMmc3 (SEQ ID NO:4); ShMmc3 (SEQ ID NO:5); NoMmc3 (SEQ ID NO:6), No2Mmc3 (SEQ ID NO:16), Sv2Mmc3 (SEQ ID NO:17), Rz2Mmc3 (SEQ ID NO:20) have a spacing of greater than about 100 amino acids or greater than 125 amino acids (and can be greater than 150 amino acids, greater than 175 amino acids, or greater than 180 amino acids) and less than about 500 amino acids, less than 450 amino acids, less than 400 amino acids, or less than about 350 amino acids between the RuvC I and RuvC II motifs. Additionally, the Mmc3 effectors disclosed herein have a spacing between the RuvC II and RuvC III motifs of greater than about 40 amino acids, or greater than about 50, 60, 70, 80, or 90 amino acids but less than about 225 amino acids, less than about 200 amino acids, or less than about 175 amino acids.

TABLE 4
Spacing of RuvC domain motifs in Type V Effector sub-types
(number of amino acid residues between motifs)

| | N-terminal | Between | Between | | |
|---|---|---|---|---|---|
| | to | RuvC I | RuvC II | C-terminal to | Total |
| | RuvC I | and RuvC II | and RuvC III | RuvC III | length |
| Mmc3 | 773 | 225 | 130 | 10 | 1181 |
| Cpf1 | 885 | 72 | 247 | 36 | 1288 |
| C2c1 | 586 | 277 | 123 | 141 | 1174 |
| C2Cc3 | 937 | 79 | 185 | 17 | 1264 |
| CasX | 659 | 79 | 154 | 44 | 982 |
| CasY | 885 | 70 | 147 | 42 | 1190 |
| Cas9 | 6 | 739 | 211 | 364 | 1349 |

The consensus sequences of the catalytic RuvC motifs are listed in Table 5. The residues necessary for catalysis (D at position 6 in RuvC I, E at position 5 in RuvC II, and D at position 5 in RuvC III) are conserved in every example Amino acid position numbers for the Mmc3 RuvC I, RuvC II, and RuvC III motifs are as shown in FIG. 5. While the overall conservation in these motifs is similar between different sub-types, there are noticeable differences that are consistent with designation of Mmc3 as a distinct Type V CRISPR system.

In RuvC I, amino acids with large, hydrophobic side chains are conserved at position 1 for Mmc3, while other sub-types have amino acids with polar, uncharged (Cpf1, C2c3, and CasX) side chains or positively charged side chains (C2c1 and CasY). Mmc3 effectors show variation at several residues in RuvC I (position 7, 9 and 10) that are highly conserved in effectors of other sub-types. For example, at position 7, Cpf1 and CasX have a conserved arginine, C2c1, C2c3, CasY, and Cas9 have hydrophobic amino acids, while Mmc3 can have either. Mmc3 effectors also have a conserved glutamic acid or glutamine at RuvC I position 11 which is not seen in any of the other sub-types. In addition, position 14 of RuvC I is in all cases but one (NapMmc3) threonine or serine followed by a leucine at position 15, a combination not seen in other effector subtypes. With respect to RuvC II, in Mmc3 effectors, position 6 is a conserved aspartate in half the sequences and unconserved in the others while other sub-types have stricter conservation at this position (negatively charged amino acids in Cpf1 and C2c1, hydrophobic amino acids in CasY and Cas9). In RuvC III, some of the Mmc3 effectors have a histidine at position 2, which is only seen in Cas9 and has been shown to be involved in catalysis in other RuvC proteins. Some Mmc3 have aspartic acid at RuvC III position 6, which is not seen in any other sub-types, although C2c1 and CasX have glutamic acid at this position. At RuvC III position 7, Mmc3 effectors have either hydrophobic or polar, uncharged amino acids, while the other sub-types have one or the other (Cpf1, C2c1, C2c3, CasX, and CasY have polar, uncharged amino acids and Cas9 has hydrophobic amino acids). Most Mmc3 have a glutamic acid at RuvC III position 18, which is not seen in any other sub-type. Overall, Mmc3 effectors show unique RuvC domain spacing and consensus sequences relative to other known class 2 CRISPR effectors. Table 5 provides consensus sequences of Class 2 CRISPR effector RuvC catalytic motifs and surrounding residues.

TABLE 5
Consensus sequences of RuvC subdomains

| Sub-type | RuvC I | RuvC II | RuvC III |
|---|---|---|---|
| Mmc3 | XXXGIDXGXXELATLCV | XIXLEXL | XXXXDXXAAYNIAKXGXE |
| | (SEQ ID NO: 59) | (SEQ ID NO: 60) | (SEQ ID NO: 61) |
| Cpf1 | XIIGIDRGERNLLYXXX | IXVLEDL | PXDADANGAYXIALKGLX |
| | (SEQ ID NO: 62) | (SEQ ID NO: 63) | (SEQ ID NO: 64) |
| C2c1 | RVMSVDLGXRXAAAXSV | LILFEDL | XXHADINAAQNLQXRFWX |
| | (SEQ ID NO: 65) | (SEQ ID NO: 66) | (SEQ ID NO: 67) |
| C2c3 | XIVAIDLFEXXXGYAVF | FPVLEXX | XXHADENAAINIGRXYLX |
| | (SEQ ID NO: 68) | (SEQ ID NO: 69) | (SEQ ID NO: 70) |
| CasX | NLIGXDRFENIPAVIAL | XLXFENL | EXHADEQAALNIARSWLF |
| | (SEQ ID NO: 71) | (SEQ ID NO: 72) | (SEQ ID NO: 73) |
| CasY | XYXGIDIFEYGXAXXXX | KXXYEXE | XXDADIQAXXXIAXXXYX |
| | (SEQ ID NO: 74) | (SEQ ID NO: 75) | (SEQ ID NO: 76) |
| Cas9 | YSIGLDIGTNSVGWAVX | XIVVEMA | HHAHDAYLNAVIGXALLK |
| | (SEQ ID NO: 77) | (SEQ ID NO: 78) | (SEQ ID NO: 79) |

In addition to the distinct consensus sequences across all three RuvC subdomains, Mmc3 sequences share a unique positioning and spacing of these subdomains (Table 6). Generally, among the 21 Mmc3 effector sequences listed in Table 6, the RuvC I subdomain (17 residues) is found at amino acid position 650-900 range from the N-terminus, followed by a spacer amino acid stretch of 125-350 residues, followed by the RuvC II subdomain (7 residues), followed by a spacer amino acid stretch of 25-225 residues, followed by the RuvC III subdomain (18 residues) which is very proximal to the C-terminus (<25 residues).

TABLE 6
Analysis of positioning and spacing of domains of
Mmc3 effectors (amino acids)

| | | | Between | | |
|---|---|---|---|---|---|
| | | Between | RuvC II | | |
| | N-terminal | RuvC I and | and | C-terminal | |
| | to RuvC I | RuvC II | RuvC III | to RuvC III | Total |
| SfMmc3 (SEQ ID NO: 2) | 869 | 212 | 162 | 12 | 1297 |
| SvMmc3 (SEQ ID NO: 3) | 838 | 215 | 171 | 19 | 1285 |
| BdMmc3 (SEQ ID NO: 1) | 835 | 204 | 147 | 12 | 1240 |
| SfpMmc3 (SEQ ID NO: 15) | 842 | 199 | 143 | 6 | 1232 |
| Smp2Mmc3 (SEQ ID NO: 12) | 839 | 222 | 109 | 6 | 1217 |
| NoMmc3 (SEQ ID NO: 6) | 727 | 301 | 98 | 5 | 1172 |
| ShMmc3 (SEQ ID NO: 5) | 744 | 299 | 97 | 6 | 1187 |
| CrpMmc3 (SEQ ID NO: 13) | 691 | 210 | 110 | 14 | 1067 |
| ObpMmc3 (SEQ ID NO: 14) | 722 | 197 | 106 | 1 | 1067 |
| NapMmc3 (SEQ ID NO: 4) | 667 | 209 | 117 | 0 | 1033 |
| SmpMmc3 (SEQ ID NO: 11) | 695 | 206 | 115 | 6 | 1064 |
| Smp3Mmc3 (SEQ ID NO: 10) | 695 | 210 | 132 | 5 | 1084 |
| PcMmc3 (SEQ ID NO: 7) | 841 | 212 | 162 | 12 | 1269 |
| Sv2Mmc3 (SEQ ID NO: 17) | 842 | 215 | 172 | 19 | 1290 |
| No2Mmc3 (SEQ ID NO: 16) | 735 | 301 | 96 | 4 | 1177 |
| No3Mmc3 (SEQ ID NO: 26) | 731 | 302 | 98 | 7 | 1180 |
| RzMmc3 (SEQ ID NO: 22) | 690 | 208 | 109 | 6 | 1055 |
| Rz2Mmc3 (SEQ ID NO: 20) | 670 | 203 | 107 | 8 | 1030 |
| Sv3Mmc3 (SEQ ID NO: 24) | 830 | 214 | 172 | 39 | 1297 |
| Sf4Mmc3 (SEQ ID NO: 23) | 872 | 190 | 144 | 10 | 1258 |
| Sf8Mmc3 (SEQ ID NO: 25) | 863 | 211 | 163 | 12 | 1291 |
| Average | 773 | 225 | 130 | 10 | 1181 |
| Lowest | 667 | 190 | 96 | 0 | 1030 |
| Highest | 872 | 302 | 172 | 39 | 1297 |

The success in identifying all three RuvC catalytic motifs prompted searching for other functional domains in Mmc3. As discussed below, Blastp analysis using Mmc3 sequences as queries exclusively returned full length hits to other Mmc3 proteins. However, BLAST searching using BdMmc3, SvMmc3, and SfpMmc3 as queries returned a small number of low quality partial hits to Cpf1 (Evalue >1e-6) sequences at a central portion of Mmc3 (AAs ˜650-850). This portion encompasses the RuvC I region and is approximately 150 amino acids N-terminal to the RuvC I motif. In Cpf1, this region includes the WED domain that is responsible for nucleotide-specific interactions with the 5′ handle of the crRNA (Yamano et al. 2016). Many of the residues in this region that directly interact with the crRNA are highly conserved among Cpf1 effectors. Given that Mmc3 likely utilizes a crRNA with a 5′-handle sequence (corresponding to the sequence at the 3′ end of the CRISPR repeat (see FIG. 3A) similar to Cpf1, sequence conservation in the WED domain might be anticipated. To examine this possibility, a multiple alignment of all Mmc3 effectors was assessed for conservation of key conserved residues in the Cpf1 WED domain. Overall, alignment of Mmc3 sequences to the Cpf1 WED domain was of poor quality. Where alignment was of sufficient quality to facilitate comparison, there was no finding of conservation of WED domain residues implicated in direct crRNA interaction. These results indicate that the low quality, partial alignments to Cpf1 for a subset of Mmc3 effectors was not predictive of conservation of the WED domain. Further, although Mmc3 utilizes a similar crRNA repeat sequence as Cpf1 (see Example 3), the mechanism by which the crRNA interacts with the effector is likely different due to the different domain structure/protein fold. Finally, Yamano et al. identified a second nuclease domain in AsCpf1 referred to as the nuc domain This second nuclease domain has so far only been reported for Cpf1, so is a defining feature of that family No support for alignment of the Cpf1 nuc domain to Mmc3 was obtained using methods similar to those described for RuvC and the WED domain.

### Zinc Finger Domain

During manual inspection of an alignment of Mmc3 sequences, it was noticed that there are four conserved cysteine residues near the C-terminus of Mmc3 between the RuvC II and III domains (FIG. 6). The conserved cysteines form two pairs, the first of which includes two cysteine residues separated by 2 intervening residues and the second of which is separated by 2-5 intervening residues. There are between 11 and 48 residues between the second cysteine of the first pair and the first cysteine of the second pair. For example, BdMmc3 has the cysteines of the first pair at amino acid positions 1171 and 1174 and the cysteines of the second pair at amino acid positions 1188 and 1193; NoMmc3 has the cysteines of the first pair at amino acid positions 1123 and 1126 and the cysteines of the second pair at amino acid positions 1138 and 1142; SfMmc3 has the cysteines of the first pair at amino acid positions 1205 and 1208 and the cysteines of the second pair at amino acid positions 1249 and 1252; and SvMmc3 has the cysteines of the first pair at amino acid positions 1178 and 1181 and the cysteines of the second pair at amino acid positions 1230 and 1233. The grouping of two pairs of cysteines is characteristic of zinc finger protein structural motif. The cysteine pairs of zinc finger domains coordinate metal ions, usually zinc, and are often involved in binding to DNA, RNA, or other molecules. Hidden Markov model searches of the Mmc3 zinc finger region show that it is most similar to zinc finger domains in the Zinc Beta Ribbon clan but does not exactly match any pfam in the clan. Several Class 2 CRISPR-Cas effectors have zinc finger domains located between the RuvC II and III domains near the C-terminus (Shmakov et al. (2017) Nat Rev Microbiol. 15: 169-182). Type V effectors C2c3, CasX, and CasY all have zinc finger domains whereas C2c1, Cpf1, and Type II effector Cas9 are characterized by Shmakov et al. (2017, ibid) as having lost or inactivated their zinc finger domains.

### Evolutionary Relationship to Other TypeV Systems

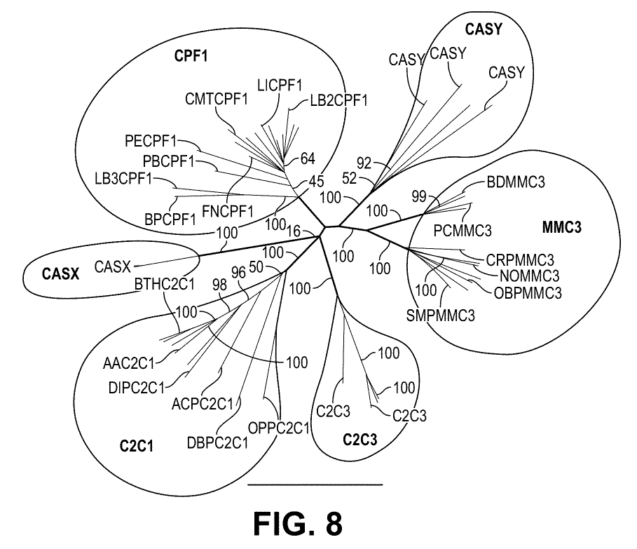

Mmc3 protein sequences were aligned with reference sequences for other known Type V CRISPR systems, namely, Cpf1, C2c1, CasY, CasX, and C2c3. Cpf1 reference sequences were taken from Zetsche et al. (2015), as these sequences were considered representative of Cpf1 sequence diversity across the family, and these are the only Cpf1s to date that have been functionally characterized. Reference C2c1 and C2c3 sequences were taken from Shmakov et al. 2015, and the CasX and CasY sequences were taken from Burstein et al. 2017 Nature 542: 237-541. All subsequent phylogenetic analyses were performed using Geneious R10 software (Geneious.com). Multiple sequence alignments were constructed using MUSCLE with default settings and up to 10 iterations (Edgar et al. 2004 Nucl Acids Res., 32: 1792-1797). Alignments were used in conjunction with PHYML (atgc-montpellier.fr/phyml/) to construct a phylogenetic tree based on a maximum-likelihood model with 100 pseudo-replicates. High-support was recovered for Mmc3 representing a distinct mono-phyletic clade within Type V CRISPR systems, as indicated by the bootstrap analysis showing 100% of all pseudo-replicates supporting the Mmc3 clade (see FIGS. 7 and 8).

The relationship between Cpf1 and Mmc3 was investigated further by performing an All-by-All Blast of the effector protein sequences. For this, BLAST+ v.2.2.31 was used to generate a BLAST protein database for Cpf1, Mmc3, C2c1, C2c3, CasX, and CasY. The FASTA file used to make the database was then aligned against itself, returning in all possible alignments. The e-values from the alignments were used to make a network file, in which each protein was a node, and the lowest e-value for each query-subject pair were the edges. As each protein also served as a query sequence, nodes typically have two edges between them. This network was visualized in Cytoscape 3.4.0., using a circular layout and the “bundle edges” function for readability. Using a threshold of 1e-15 all Mmc3 effectors cluster as a unique group amongst other Type V systems. Raising this threshold to 1e-14 maintains Mmc3 as a distinct group, but results in an edge between C2c3 and CasY systems, which are established in the literature as distinct CRISPR subtypes (Burstein et al. 2017). A threshold of 1e-11 is required to disrupt Mmc3 clustering, but at this threshold numerous edges are now present between C2c3 and CasY. Together, this analysis supports the claim the Mmc3 is distinct effector and a new sub-type of TypeV CRISPR systems.

### Blastp Analysis of Mmc3 Against NCBI NR Database

Each Mmc3 effector protein sequence was used as a query against the NCBI non-redundant database using the Blastp algorithm with default settings. Overall, the top hits to Mmc3 queries were other Mmc3 polypeptides, as defined by prior phylogenetic and Blastp network analyses. Several Mmc3 queries returned hits to Cpf1 annotated proteins, but these hits were only to a small fraction of the Mmc3 query sequence, and typically had high expectation of occurring by chance with E-value >0.01. These results support the claim that Mmc3 is evolutionarily distinct relative to known Type V effector families.

Table 7 summarizes the findings, showing hits returning E-values >0.01 ranked by % query coverage.

TABLE 7
Mmc3 queries against the NCBI nr database

| Query | Accession | % ID | Query Coverage | E-value | Description |
|---|---|---|---|---|---|
| NoMmc3 | KIE18642.1 | 32% | 94% | 1.E−111 | Smp3Mmc3 |
| SEQ ID NO: 6 | KFO67988.1 | 33% | 91% | 4.E−108 | SmpMmc3 |
| | OGX23684.1 | 30% | 89% | 2.E−75 | ObpMmc3 |
| | KKQ38176.1 | 29% | 83% | 7.E−67 | CrpMmc3 |
| SfMmc3 | KIM12007.1 | 35% | 98% | 0.E+00 | SfpMmc3 |
| SEQ ID NO: 2 | KIE18642.1 | 24% | 58% | 1.E−17 | Smp3Mmc3 |
| | OGX23684.1 | 23% | 58% | 9.E−12 | ObpMmc3 |
| | KKQ38176.1 | 20% | 57% | 3.E−08 | CrpMmc3 |
| | KFO67988.1 | 23% | 52% | 1.E−12 | SmpMmc3 |
| BdMmc3 | KIM12007.1 | 43% | 99% | 0.E+00 | SfpMmc3 |
| SEQ ID NO: 1 | KKQ38176.1 | 22% | 58% | 6.E−09 | CrpMmc3 |
| | OGX23684.1 | 24% | 32% | 1.E−04 | ObpMmc3 |
| | OGW03971.1 | 25% | 18% | 8.E−04 | Cpf1 |
| SvMmc3 | KIM12007.1 | 35% | 98% | 0.E+00 | SfpMmc3 |
| SEQ ID NO: 3 | OGX23684.1 | 23% | 61% | 6.E−11 | ObpMmc3 |
| | KIE18642.1 | 24% | 58% | 3.E−13 | Smp3Mmc3 |
| | KKQ38176.1 | 22% | 50% | 5.E−13 | CrpMmc3 |
| | WP_016301126.1 | 26% | 19% | 0.005 | Cpf1 |
| | WP_009217842.1 | 26% | 18% | 8.E−04 | Cpf1 |
| | SER03894.1 | 26% | 18% | 0.002 | Cpf1 |
| | WP_006283774.1 | 26% | 16% | 0.002 | Cpf1 |
| NapMmc3 | KIE18642.1 | 37% | 99% | 0.E+00 | Smp3Mmc3 |
| SEQ ID NO: 4 | KFO67988.1 | 36% | 99% | 4.E−179 | SmpMmc3 |
| | KKQ38176.1 | 31% | 99% | 2.E−116 | CrpMmc3 |
| | OGX23684.1 | 30% | 99% | 6.E−119 | ObpMmc3 |
| ShMmc3 | KIE18642.1 | 33% | 92% | 6.E−113 | SmpMmc3 |
| SEQ ID NO: 5 | KFO67988.1 | 34% | 89% | 2.E−116 | Smp3Mmc3 |
| | KKQ38176.1 | 27% | 74% | 3.E−67 | CrpMmc3 |
| | OGX23684.1 | 30% | 73% | 1.E−91 | ObpMmc3 |
| SmpMmc3 | KIE18642.1 | 80% | 99% | 0.E+00 | Smp3Mmc3 |
| SEQ ID NO: 11 | OGX23684.1 | 32% | 99% | 1.E−157 | ObpMmc3 |
| | KKQ38176.1 | 29% | 99% | 4.E−113 | CrpMmc3 |
| CrpMmc3 | OGX23684.1 | 31% | 98% | 1.E−123 | ObpMmc3 |
| SEQ ID NO: 13 | KIE18642.1 | 29% | 98% | 1.E−115 | Smp3Mmc3 |
| | KFO67988.1 | 29% | 98% | 9.E−113 | SmpMmc3 |
| | KIM12007.1 | 22% | 65% | 4.E−08 | SfpMmc3 |
| ObpMmc3 | KFO67988.1 | 32% | 98% | 6.E−159 | SmpMmc3 |
| SEQ ID NO: 14 | KIE18642.1 | 32% | 98% | 2.E−154 | Smp3Mmc3 |
| | KKQ38176.1 | 31% | 97% | 5.E−125 | CrpMmc3 |
| | KIM12007.1 | 23% | 69% | 5.E−10 | SfpMmc3 |
| | WP_015940869.1 | 35% | 6% | 0.008 | transposase |
| SfpMmc3 | OGX23684.1 | 22% | 61% | 2.E−08 | ObpMmc3 |
| SEQ ID NO: 15 | KKQ38176.1 | 22% | 56% | 5.E−08 | CrpMmc3 |
| | WP_066040075.1 | 25% | 28% | 3.E−05 | Cpf1 |
| | KIE18642.1 | 24% | 25% | 5.E−04 | Smp3Mmc3 |
| | OGW03971.1 | 26% | 21% | 1.E−06 | Cpf1 |
| | WP_006283774.1 | 24% | 20% | 2.E−04 | Cpf1 |
| | SER03894.1 | 24% | 20% | 2.E−04 | Cpf1 |
| | KFO67988.1 | 25% | 17% | 4.E−04 | SmpMmc3 |
| | WP_065256572.1 | 29% | 16% | 8.E−05 | Cpf1 |
| | WP_036388671.1 | 29% | 16% | 1.E−04 | Cpf1 |
| | WP_049895985.1 | 29% | 16% | 2.E−04 | Cpf1 |
| | WP_062499108.1 | 28% | 16% | 1.E−04 | Cpf1 |
| | WP_024988992.1 | 28% | 15% | 0.006 | Cpf1 |
| | WP_016301126.1 | 26% | 15% | 9.E−04 | Cpf1 |

A BLAST search using NoMmc3 as query recovered four hits. The four hits are Mmc3 proteins: SmpMmc3, Smp3Mmc3, ObpMmc3 and CrpMmc3 (Table 7). A BLAST search using SfMmc3 as query resulted in five hits of high significance (E-value <1e-8) which are Mmc3 proteins: SfpMmc3, Smp3Mmc3, SmpMmc3, ObpMmc3, and CrpMmc3 (Table 7); two hits were obtained having low significance (E-value >0.01) and these two hits are annotated Cpf1 sequences: OGD68774.1, OGF20863.1. Both hits are to the RuvC domain and account for only 15% of the Mmc3 Query sequence. Based on full length alignments both hits are ˜11% ID to SfMmc3.

Using BdMmc3 as the query, three hits were recovered having high significance (E-value <1e-4), which were all Mmc3 proteins: SfpMmc3, CrpMmc3 and ObpMmc3. SfpMmc3 was the only hit spanning the entire length of query BdMmc3. A single hit with E-value 8e-4 was obtained to annotated Cpf1 protein OGW03971, however, this hit was based on only 18% query coverage. OG03971 shares only 12% amino acid identity to BdMmc3 when aligned over the full length of the protein. Four hits of low significance (E-value >0.01) were to Cpf1 annotated proteins with query coverage of ˜16%. In addition, there were several transposase domain proteins (Tn orfB) proteins which showed a small region of similarity with the BdMmc3 RuvC domain (E-value >0.01).

Using SvMmc3 as a query, four Mmc3 proteins: SfpMmc3, CrpMmc3, ObpMmc3 and SfpMmc3, were recovered having E-value <1e-5. Of these, only SfpMmc3 aligned to SvMmc3 along its entire length. Four hits having E-value <0.01 & >1e-5 to Cpf1 annotated proteins were recovered. One of the recovered proteins, WP_009217842.1 showed 18% query coverage and E-value=8e-04. Another protein recovered in this Blast search was SER03894.1, showing 16% query coverage & E-value=0.002. Thirteen proteins were recovered as hits of low significance (E-value >0.01) and were identified as Cpf1 annotated proteins, with query coverage of 11% -17%.

BLAST-search recovery results with NapMmc3 as a query Resulted in four hits with of high significance (E-value <1e-116) that were all Mmc3 proteins: SfpMmc3, Smp3Mmc3, CrpMmc3, and ObpMmc3. Five hits with E-values >0.01 were orfB family transposases.

A BLAST-search using ShMmc3 as a query retrieved the sequences of Smp3Mmc3, SmpMmc3, ObpMmc3, CrpMmc3 as top hits (E-value <1e-67).

BLAST-search recovery results with SmpMmc3 as a query resulted in hits of high significance (E <1e-113) that were Mmc3 proteins SfpMmc3, Smp3Mmc3, CrpMmc3, and ObpMmc3. Several low quality hits were recovered to orfB family transposases (E-value >0.01).

BLAST analysis results with CrpMmc3 as a query retrieved four hits with E-value <1 e-8 that were all Mmc3 proteins: SfpMmc3, SmpMmc3, Smp3Mmc3, and ObpMmc3.

BLAST results using ObpMmc3 as a query showed recovery of significant hits to SfpMmc3, SmpMmc3, Smp3Mmc3, and CrpMmc3 (E-value <1e-10). Several low quality hits were recovered to proteins with transposase E-value >0.01.

BLAST results using SfpMmc3 as a query with highest significance were CrpMmc3 and ObpMmc3 (Evalue <1e-6). Smp3Mmc3 and SmpMmc3 (E >1e-6) and 17-25% query coverage were also recovered. Several hits were recovered to Cpf1 proteins (E-value >1e-6), showing about 12-28% query coverage. Cpf1 hits spanned the RuvC I sub-domain and the WED domain Cpf1-annotated protein OGW0397.1 was again recovered in this search, having 21% query coverage and E-value=1e-6. Cpf1-annotated protein WP_066040075.1 was also recovered, having 28% query coverage & E-value=3e-5. In both cases, alignment of the full-length proteins showed ˜12% identity to SfpMmc3.

Based on the results provided above, we conclude that the polypeptides designated as Mmc3 do not share substantial sequence similarity with any other protein class or effector proteins other than with themselves. The few results which identified a Cpf1 family member were typically derived from low quality alignments (E-value >0.01) covering a small region of the query sequence (<20%). Cpf1 hits aligned to the RuvC domain and/or the WED domain, which might be expected based on the conservation of the RuvC domain across all Type V systems and the putative role of the WED domain in interacting with the crRNA 5′ handle. However, when examined across the entire set of Cpf1 and Mmc3 reference sequences, the RuvC and WED domains show substantial differences in conservation of key residues that reinforces the lack of broader sequence homology, demonstrating that Mmc3 and Cpf1 are different protein families

### Example 2

### Depletion Libraries and PAM Analysis

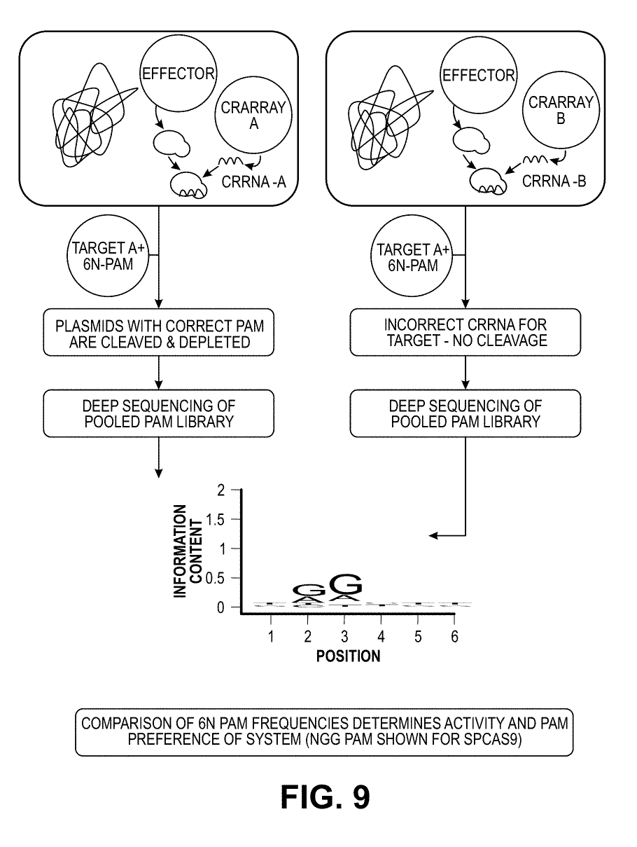

The protospacer adjacent motif (PAM) is a typically 2-6 base pair DNA sequence immediately proximal to the DNA sequence targeted by the nuclease (protospacer). Depending on the CRISPR system, a PAM sequence can be positioned either 5′ or 3′ relative to the protospacer sequence. Type V CRISPR-Cas systems show a specificity towards 5′ PAM sequences that are T-rich. In contrast, Cas9, a Type II Cas, has specificity for a 3′ G-rich PAM sequence. Plasmid depletion studies were performed as means to demonstrate activity of Mmc3 systems and determine their PAM sequence requirement. The general workflow for these experiments is given in FIGS. 9 and 10. Briefly, cells expressing the effector and either a targeting CRISPR array (‘CRarray A’ in FIG. 9) or a non-targeting CRISPR array (control, ‘CRarray B’ in FIG. 9) are made competent for further transformation with the target (protospacer-containing) plasmid. Cells of each type (targeting ‘CRarray A’-containing and non-targeting ‘CRarray B’-containing) are then transformed with a plasmid library that includes all combinations of a 5′-6N PAM sequence (i.e., a 5′-6N PAM library) juxtaposed with the protospacer that matches the spacer in the targeting CRISPR array (‘Target A+6N-PAM’ in FIG. 9). For testing of Mmc3 effectors, a 5′ PAM library was used as all Type V systems described to date require a 5′ PAM sequence. In this scheme, systems showing RNA-guided DNA interference will cleave the subset of plasmids with the correct protospacer and PAM sequence, thereby depleting these plasmids from the transformed population. For the non-targeting CRISPR array control, cleavage of the target plasmid should not occur, regardless of the PAM sequence on the plasmid, so no selective depletion of any PAM sequence in the transformed population is expected. After recovery of transformants and deep sequencing of target plasmids, the frequency of each of the PAM sequences found in the transformants is compared between targeting and non-targeting experiments. Identification of PAM sequence motifs depleted in the targeted population relative to the non-targeted population indicates these PAM sequences promoted successful RNA-guided DNA-interference (cleavage and removal of the plasmid), allowing inference of the system's PAM preference.

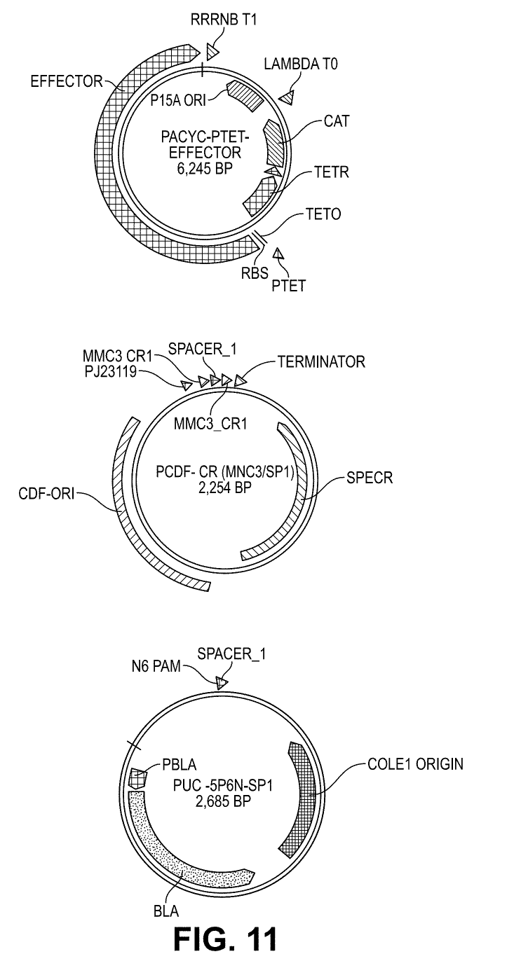

FIG. 11 provides diagrams of plasmid constructs used to test programmable DNA cleavage of Mmc3 systems and determine PAM preferences. As shown in FIG. 11, the test system outlined in FIG. 9 has three genetic components: 1) a synthesized effector gene cloned into a low copy vector under the control of an inducible Ptet promoter, 2) a synthetic minimal CRISPR array encoding a non-natural spacer sequence (‘Spacer 1’ (SEQ ID NO:82) positioned between CR repeats, and 3) a target plasmid with the specific protospacer (‘Spacer 1’, SEQ ID NO:82) and either a 5′ 6N-PAM library or a specific 5′-PAM sequence. FIG. 9 shows the schematics of a depletion assay based on these plasmids for quantifying targeted DNA cleavage activity of CRISPR/Cas and determining PAM preferences using a 6N PAM library, and FIG. 10 shows a flowchart of the overall process. Testing each Mmc3 member for its PAM specificity is accomplished by assessing the ability of a nuclease to cleave a library of plasmids containing a protospacer flanked by a random 6-mer PAM sequence. In this system, plasmids having PAM sequences that support effector nuclease activity are cleaved and thereby depleted from the resulting transformed population because the the host carrying a plasmid with the functional PAM is not viable—thus the function of the PAM is confirmed by its depletion from the transformed population. PAM libraries can be synthesized, and a a panel of 6-mers representing the various permutations of each of the four nucleotide residues at each position of the PAM can be juxtaposed to a synthesized target (protospacer) sequence in a construct that is transformed into the test cells to determine the specificity and nuclease activity for each Mmc3 family member.

To identify the PAM sequence used by Mmc3 effectors, a synthetic spacer sequence (‘Spacer 1’, SEQ ID NO:82) that would serve as the target sequence for cleavage was cloned into a pUC-based plasmid backbone that included a colE1 origin of replication and a beta-lactamase gene conferring resistance to beta lactam antibiotics. A N6 PAM library was constructed by inserting a random 6-mer immediately 5′ of the spacer sequence. To do this, an inverse PCR reaction was performed using a forward primer than binds to Spacer1 and a reverse primer that binds upstream of Spacer1on the reverse strand. The reverse primer included six random bases at the 5′ end followed by Spacer1sequence. The resultant product was covalently closed using Gibson Assembly and cloned into E. coli. A diverse number of resultant clones (>100,000 CFU) were recovered and used to prepare plasmid DNA.

Preparation of CRISPR-array and Mmc3 transformed bacteria for testing the functionality of PAM sequences was as follows: EPI-300 electrocompetent bacteria were transformed with the effector plasmid and the CRISPRarray plasmid combinations to be tested. Co-transformants were selected on LB plates supplemented with 12.5 μg/mL chloramphenicol (Cm12.5) to select for the effector expression plasmid plus 50 μg/uL spectinomycoin (Sp50) to select for the CRISPR array plasmid and incubated overnight at 37° C. On the following day, 3 mL LB+Cm12.5 plus Sp50 was inoculated with independent clones for each effector/CRISPR pairing. On the third day, overnight cultures were diluted (1:100) into 60 mL LB+Cm12.5+Sp50 +anhydrotetracycline 100 ng/mL (aTc100) (which induces expression of the effector) in a 250 mL flask and incubated at 37° C. while shaking 220 rpm until the optical density of the bacterial culture (OD₆₀₀) was between 0.4 and 0.6. When cultures reached the required OD, they were placed on ice to cool. Then, the culture was transferred to a 50 mL pre-chilled Falcon tube and centrifuged at 5000×g for 5-7 minutes. The resulting bacterial pellet wass washed in an equal volume of cold sterile distilled water and centrifuged at 5000×g for 5-7 minutes Finally, the pellet was washed in 1.5 mL of 10% ice cold sterile glycerol, pelleted and resuspended in 200 μL of 10% glycerol. Cells were then divided into 50 uL aliquots for use.

The plasmid depletion assay was performed on the same day that the competent cells were prepared. For controls with specific PAM sequences 50 uL of competent cells were transformed with 5 ng of plasmid. For N6 libraries, 50 uL of competent cells were transformed with 50 ng of plasmid, which equates to >1×10⁸ plasmids. Transformation was performed by electroporation using 0.1 cm cuvettes and under standard Biorad electroporator settings for bacteria (1.8 kV, 200Ω, 25 μF). The transformants were recovered with 700 μL of SOC media supplemented with aTc100 and incubated with shaking at 37° C. for 1 hour.

Recovery of N6-PAM library transformants: 30 mL of LB supplemented with Cm12.5+Sp50+Carbenicillum 100 μg/mL (Cb100)+aTc100 media in (125-250 mL) flasks were inoculated with 300 μL of transformed bacteria and incubated overnight with shaking at 37° C. Transformation titers were obtained by a serial dilution covering 10⁰-10⁵ in a microtiter plate and plating in replicate on an LB+Cb100 and/or LB+Cm12.5+Sp50 +Cb100+aTc100 plate. Plates were dried and incubated at 37° C. overnight.

TABLE 8
Control strains for Plasmid Depletion Assays

| | | Spacer in | |
|---|---|---|---|
| Strain | Effector | crRNA | Notes |
| AGE60 | Cas9 | Spacer 1 | Positive control for cleavage with known |
| | Streptococcus pyogenes | SEQ ID | Cas9 effector |
| | (SEQ ID NO: 80) | NO: 82 | |
| AGE39 | Cas9 | Spacer 2 | Negative control for cleavage with known |
| | Streptococcus pyogenes | SEQ ID | Cas9 effector, non-targeting RNA |
| | (SEQ ID NO: 80) | NO: 83 | |
| AGE153 | Cpf1 | Spacer 1 | Positive control for cleavage with known |
| | Acidaminococcus sp. | SEQ ID | Cpf1 effector |
| | (SEQ ID NO: 81) | NO: 82 | |
| AGE152 | Cpf1 | Spacer 2 | Negative control for cleavage with known |
| | Acidaminococcus sp. | SEQ ID | Cpf1 effector, non-targeting RNA |
| | (SEQ ID NO: 81) | NO: 83 | |

TABLE 9
PAM library and reporter plasmids

| | | Spacer in | |
|---|---|---|---|
| Strain | Reporter Plasmid | crRNA | Notes |
| AGE v37 | 3′-AGG-Spacer_1 | Spacer 1 | Cas9 3′ PAM |
| | reporter plasmid | SEQ ID NO: 82 | positive control |
| AGE v38 | 3′ N6-Spacer_1 | Spacer 1 | 3′ PAM Test library |
| | library | SEQ ID NO: 82 | |
| AGE v83 | 5′ TTTC-Spacer_1 | Spacer 1 | 5′ PAM Mmc3 |
| | reporter plasmid | SEQ ID NO: 82 | positive control |
| AGE v82 | 5′ TTTC-Spacer_2 | Spacer 2 | 5′ Mmc3 PAM, |
| | reporter plasmid | SEQ ID NO: 83 | incorrect target |
| AGE v76 | 5′ N6-Spacer_1 | Spacer 1 | 5′ PAM Test library |
| | library | SEQ ID NO: 82 | |

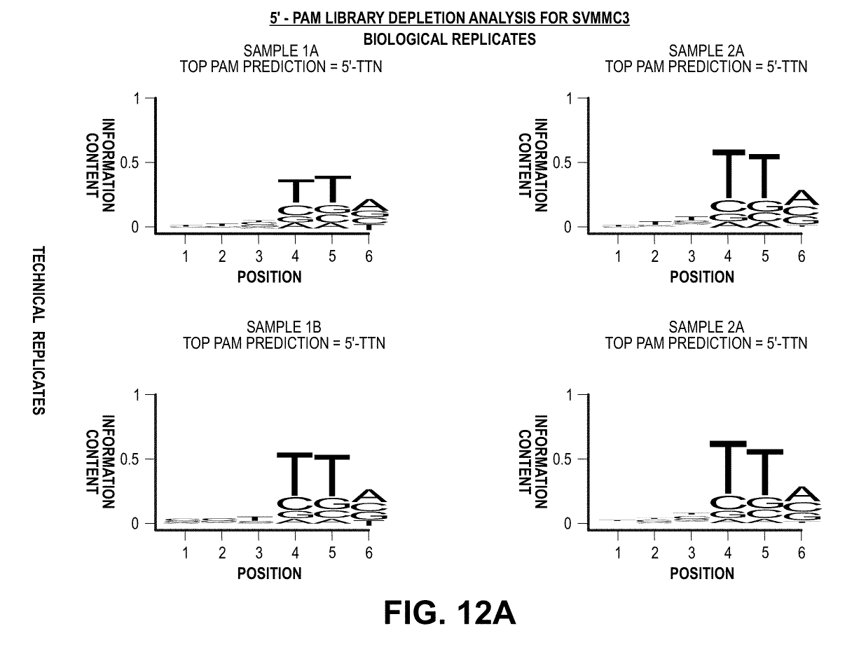

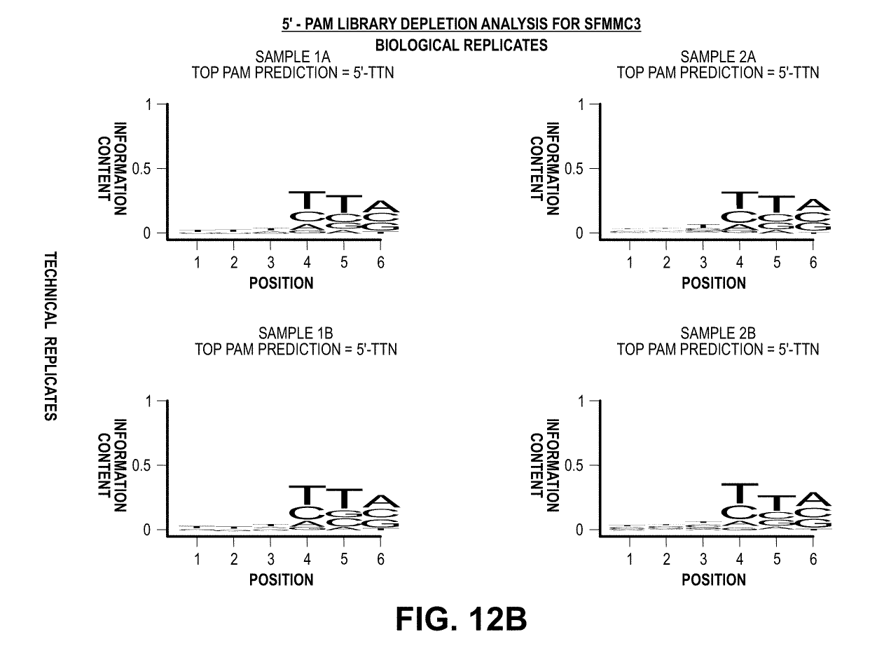

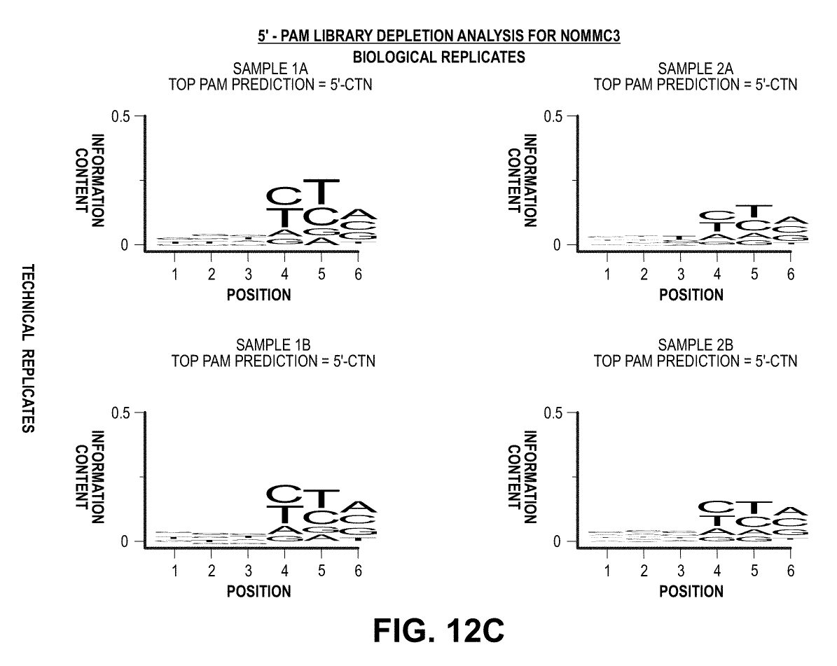

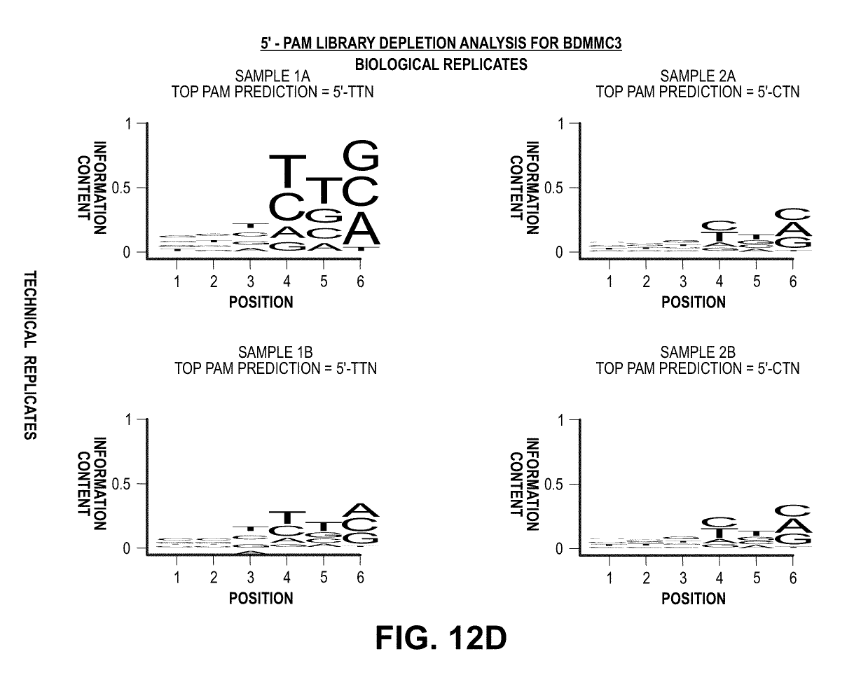

FIGS. 12A-D show PAM depletion signal results for several Mmc3 polypeptides. FIG. 12A shows PAM enrichment scores represented as SeqLogos for the Mmc3 family member SvMmc3 (SEQ ID NO:3). A 5′ TTN sequence is indicated as preferred for SvMmc3 cleavage and is consistent across both biological and technical replicates. FIG. 12B shows PAM enrichment scores represented as SeqLogos for the Mmc3 family member SfMmc3 (SEQ ID NO:2). A 5′ TTN sequence is indicated as preferred for SfMmc3 as well and is consistent across both biological and technical replicates. FIG. 12C shows PAM enrichment scores represented as SeqLogos for the Mmc3 family member NoMmc3 (SEQ ID NO:6). A 5′ CTN sequence is indicated as preferred for NoMmc3 activity and is consistent across both biological and technical replicates. FIG. 12D shows PAM enrichment scores represented as SeqLogos for BdMmc3 (SEQ ID NO:1). A 5′ CTN or 5′ TTN sequence is indicated as preferred for BdMmc3 depending on biological replicate. Results are consistent between technical replicates. In general, BdMmc3 (SEQ ID NO:1), SfMmc3 (SEQ ID NO:2) and NoMmc3 (SEQ ID NO:6) show enrichment for PAM motifs in addition to the top enriched motif suggesting the potential for more relaxed PAM requirements than SvMmc3 (SEQ ID NO:3), which has a more dominant signature for the 5′ TTN PAM enrichment.

### Example 3

### Plasmid Interference Assays to Test Genome Editing

Capabilities of Mmc3 Effectors

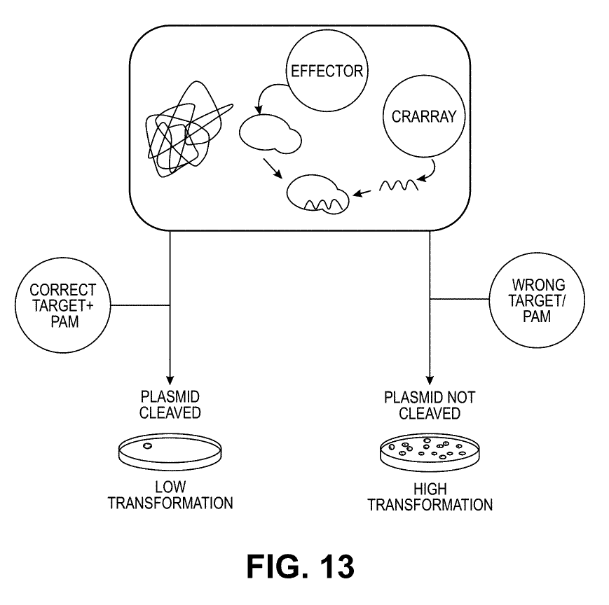

FIG. 13 depicts an assay for quantifying targeted DNA cleavage activity of CRISPR/Cas systems using target plasmids that encode specific 5′-PAM sequences flanking a compatible protospacer sequence. PAM sequences that support cleavage at the protospacer yield reduced numbers of transformants relative to controls that included a non-compatible spacer sequence in the CRISPR array (CRarray) plasmid or an incorrect PAM sequence in the target construct. This assay was performed essentially the same way as the plasmid depletion assay described in Example 2, with the exception that a PAM library plasmid was not used. Instead, as depicted schematically in FIG. 13, the system was used to test in vivo activity of a given Mmc3 effector, where plasmid depletion resulting from effector activity (cleavage of the target plasmid) was measured against a control where either an incorrect PAM or a PAM whose effectiveness was being tested was used in the target plasmid.

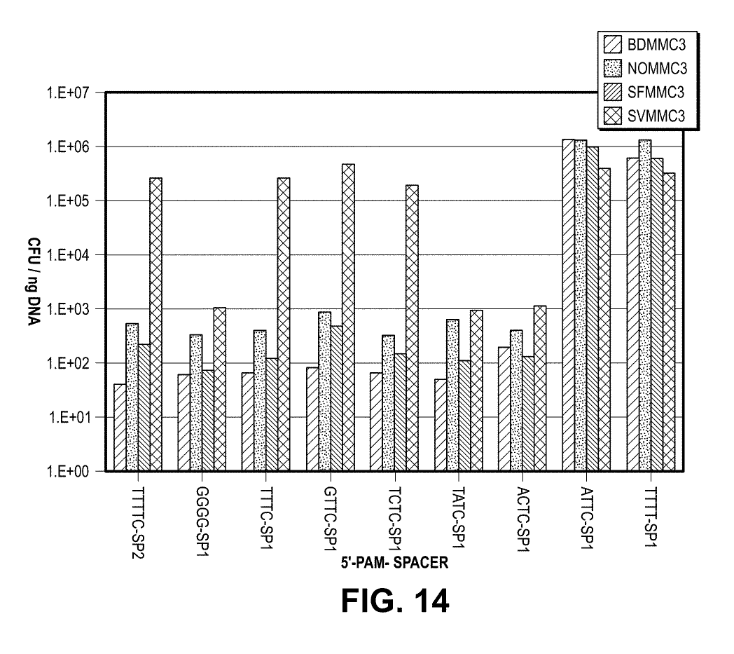

FIG. 14 illustrates the results of testing different PAM sequences when expressing BdMmc3 (SEQ ID NO:1), NoMmc3 (SEQ ID NO:6), SfMmc3 (SEQ ID NO:2), and SvMmc3 (SEQ ID NO:3) effectors using this assay system. PAM dependence of DNA interference activities for the Mmc3 systems was assessed by comparing transformation frequencies of target plasmids encoding the following 5′-PAM sequences flanking the targeted protospacer (Sp1, SEQ ID NO:82): 1) 5′-TTTT 2) 5′-ATTC 3) 5′-ACTC 4) 5′-TATC 5) 5′-TCTC 6) 5′-GTTC 7) 5′ TTTC 8) 5′ GGGG. In addition, a non-targeted protospacer (Sp2) control was performed, where the Sp2 sequence (SEQ ID NO:83) provided in the CRISPR array plasmid did not match (did not have homology to) the target sequence in the target plasmid. Relative reduction in transformation frequency compared to non-target control using a particular PAM sequence in the target plasmid indicates activity of the system for RNA-guided DNA interference using the PAM. From this analysis, BdMmc3 and NoMmc3 and SfMmc3 activity profile is consistent with a 5′-HTN PAM, where H is A, C, or T/U, whereas SfMmc3 activity profile us consistent with a 5′-TTV PAM, where V is A, C, or G. Results were largely consistent with PAM depletion analysis (see, FIGS. 12A-D), but provide finer resolution on the accepted PAM sequences for each system. For instance, SvMmc3 does not accept a ‘T’ at the first position in the PAM, whereas it was not possible to discern this from the library depletion analysis (FIG. 12A). Furthermore, SfMmc3, NoMmc3 and BdMmc3 can accept a ‘C’ at the third position in the PAM with similar efficiency to ‘T’. On examination of the depletion data it can be seen that ‘C’ and ‘T’ are enriched in the seqLogos to similar degrees for all three systems, consistent with the analysis presented (FIG. 14). In general, these analyses confirm that Mmc3 effectors have a more relaxed PAM requirement than reported for AsCpf1 and LbCpf1, which are reported to be 5′-TTTV (Kim et al. (2016) Nature Methods, 14: 153-159). A more relaxed PAM sequence is an advantage as it provides more flexible targeting options across a genome.

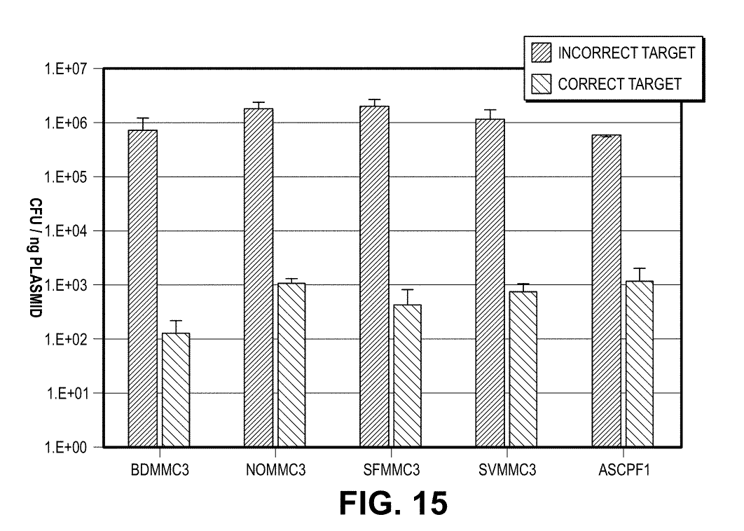

Mmc3 systems were assessed for DNA-interference activity by comparing transformation frequencies with plasmids encoding either a protospacer sequence that matched the spacer encoded by the crRNA or a non-specific protospacer sequence. The general scheme for these assays is also shown in FIG. 13. The AsCpf1 effector (SEQ ID NO:81) was also included to compare performance to another Type V CRISPR system. FIG. 15 shows target specific DNA interference activities of the Mmc3 systems relative to AsCpf1: BdMmc3 (SEQ ID NO:1), NoMmc3 (SEQ ID NO:6), SfMmc3 (SEQ ID NO:2), SvMmc3 (SEQ ID NO:3), and AsCpf1 (SEQ ID NO:81). The designation “Correct Target” indicates plasmids which encode a protospacer that matches the crRNA spacer sequence (Sp1, SEQ ID NO:82), whereas the “Incorrect Target” plasmid encode a protospacer that is mismatched with the crRNA spacer sequence (Sp2, SEQ ID NO:83). The relative reduction in transformation frequency between “Correct” and “Incorrect” target experiments indicates activity of the system for RNA-guided DNA interference. Both target plasmids encode the 5′ TTTC PAM sequence shown to support activity of Mmc3 systems and AsCpf1. From this analysis, all Mmc3 systems show 3-4 log reduction on transformation frequency for the correct target relative to the incorrect target. BdMmc3 and SfMmc3 are more active for DNA cutting in the E. coli bioassay relative to AsCpf1.

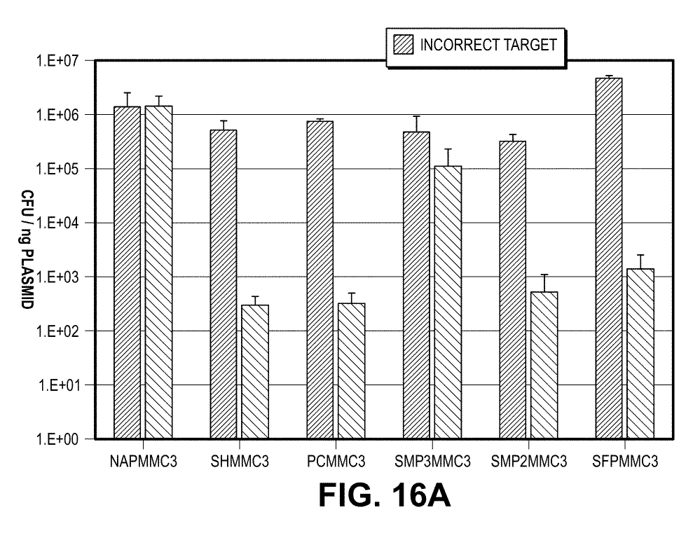

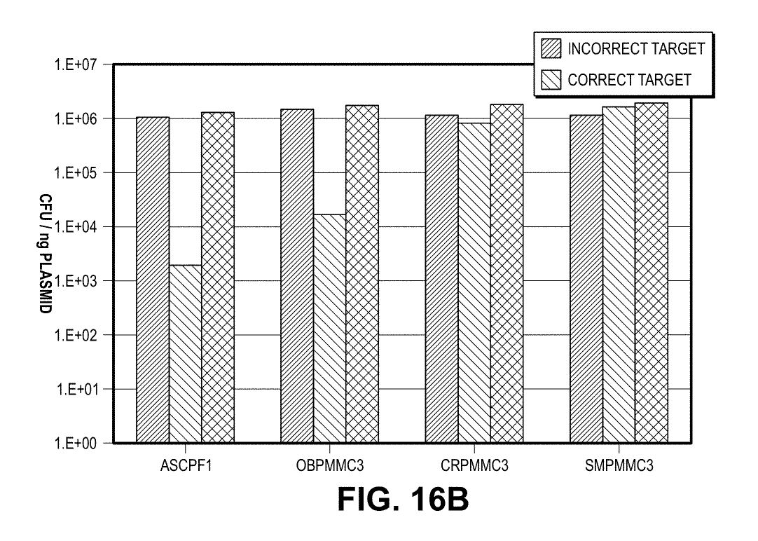

The same plasmid interference assays were also performed using NapMmc3 (SEQ ID NO:4), ShMmc3 (SEQ ID NO:5), PcMmc3 (SEQ ID NO:7), SmpMmc3 (SEQ ID NO:11), Smp2Mmc3 (SEQ ID NO:12), CrpMmc3 (SEQ ID NO:13), ObpMmc3 (SEQ ID NO:14), and SfpMmc3 (SEQ ID NO:15) effectors. Activity was observed for ShMmc3, PcMmc3, Smp2Mmc3, ObpMmc3, and SfpMmc3 utilizing Spacer 1 (Sp1) and a 5′-TTTC PAM (FIGS. 16A and 16B). As can be seen in FIGS. 15, 16A and 16B, the number of colonies resulting when the correct target sequence and PAM was used in the target plasmid dropped by at least 90% relative to controls, and by greater than three orders of magnitude for several effectors (e.g., ShMmc3, PcMmc3, SfpMmc3). The percent editing based on this plasmid interference assay for Mmc3 effectors was thus found to be at least about 90% and up to 99.9%. The activity of the ShMmc3, PcMmc3, Smp2Mmc3, and SfpMmc3 effectors compared favorably with that of known Cpf1 effector AsCpf1.

### Example 4

### Determination and Validation of Mmc3 Processed crRNA

RNA sequencing (“RNAseq”) was used to experimentally determine the sequence of processed crRNA guides for four Mmc3 systems: SfMmc3, SvMmc3, NoMmc3 and BdMmc3. Small RNA (sRNA) was purified from E. coil strains transformed with vectors expressing a particular Mmc3 effector and vectors expressing a corresponding CRISPR array having a designed spacer sequence (Sp1, SEQ ID NO:82) flanked by CRISPR repeats. As shown in FIG. 3A, the CRISPR repeat sequences of the various Mmc3 arrays are very similar to one another (consensus sequence SEQ ID NO:27), with the 3′-most 18 nucleotides of the repeats being almost identical (consensus sequence SEQ ID NO:44). Small RNA (sRNA) was prepared using the mirVana miRNA isolation kit (ThermoFisher, cat#AM1560) as described by the manufacturer. Libraries were constructed using the NEBnext small RNA library prep set (New England Biolabs) and sequenced using an Illumina MiSeq platform. After QC, trimmed reads were aligned to the Mmc3 CRISPR region and analyzed for crRNA processing.

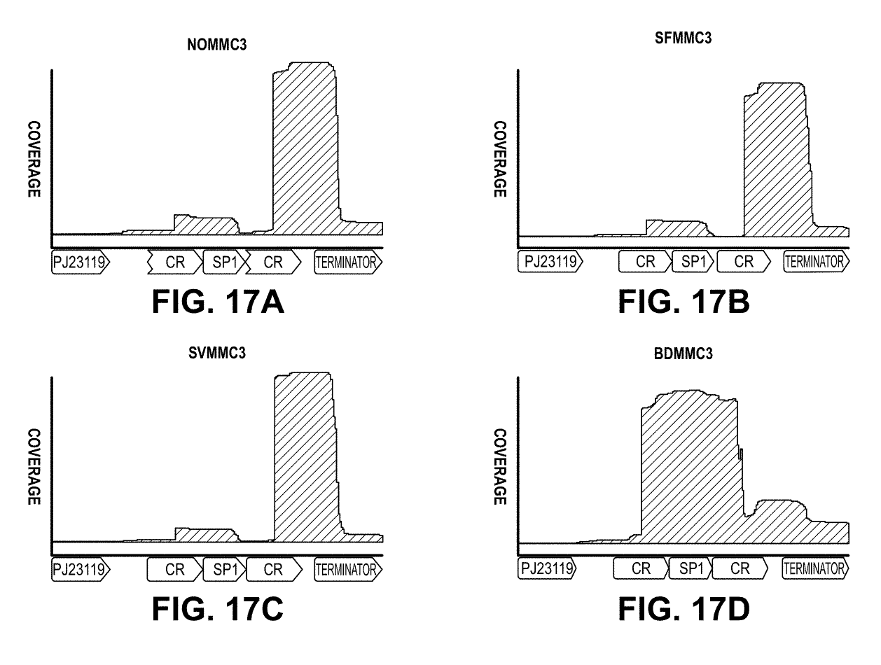

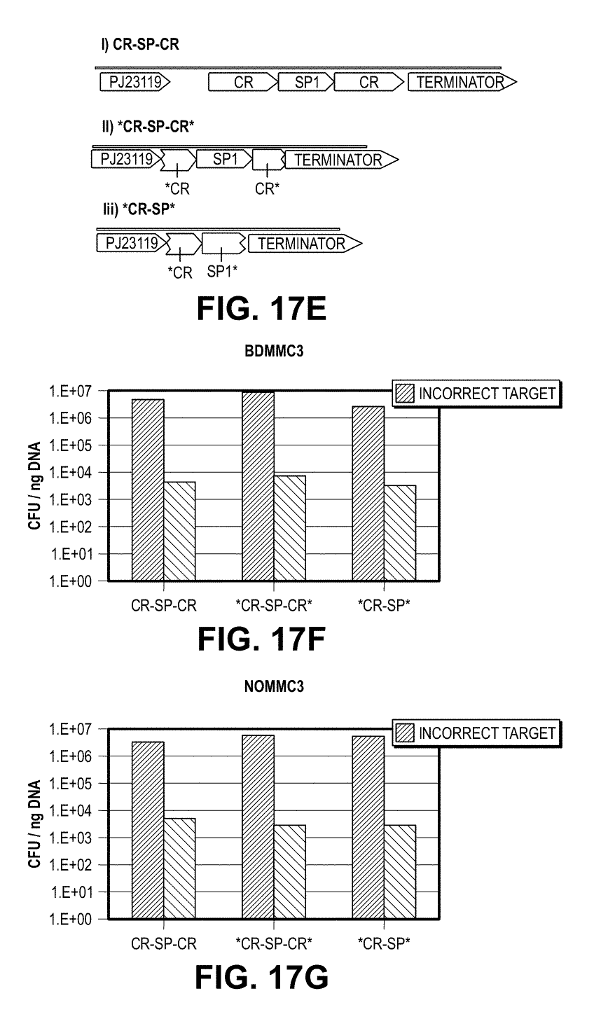

FIGS. 17A-D show diagramatically the results of the RNAseq, with reads mapped against the constructs for expressing the crRNAs. Co-expression of the crRNA and either the SfMmc3 (SEQ ID NO:2) or SvMmc effector (SEQ ID NO:3) resulted in processing of the crRNA to contain 18 bp of the CRISPR repeat sequence (SEQ ID NO:45 for SfMmc3 and SEQ ID NO:47 for SvMmc3) followed by 18-25 bp of the spacer sequence 3′ to the CRISPR repeat. The processed NoMmc3 crRNA showed a similar structure to the SfMmc3 repeat but had a 19 bp CRISPR repeat sequence (SEQ ID NO:46). Although the 3′ processing of the BdMmc3 crRNAs could not be resolved, it was empirically found that guide RNAs (crRNAs) having 18-19 nucleotides of the 3′ end of the repeat sequence juxtaposed with an 18-23 nucleotide target (spacer) sequence which was positioned 3′ of the 18-19 nucleotide repeat sequence were effective for genome editing regardless of whether the crRNA also included repeat sequences 3′ of the spacer (target) sequence.

Based on the RNAseq results, processed forms of crRNA were tested by modifying the construct that encoded the crRNA used for the E. coli plasmid interference assays. FIG. 17E shows constructs that were tested for expressing crRNAs in E. coli that expressed either the BdMmc3 (SEQ ID NO:1) or NoMmc3 (SEQ ID NO:6) effector. In the “CR-Sp-CR” construct (SEQ ID NO:86), a full CRISPR repeat (SEQ ID NO:28 for BdMmc3 and SEQ ID NO:33 for NoMmc3) was positioned downstream of the PJ23119 promoter (SEQ ID NO:84), followed by the Spacer 1 sequence (Sp1, SEQ ID NO:82), which was followed by another full CRISPR repeat (SEQ ID NO:28 for BdMmc3 or SEQ ID NO:33 for NoMmc3). A terminator sequence (SEQ ID NO:85) was positioned downstream of the second CRISPR repeat. The *CR-Sp-CR* construct (SEQ ID NO:87) had the same promoter-CRISPR repeat-Spacer-CRISPR repeat-terminator organization, but the CRISPR repeats were either an 18 nt repeat (for BdMmc3, SEQ ID NO:45) or 19 nt repeat (for NoMmc3, SEQ ID NO:46) instead of the full 36 or 37 nucleotides of the native repeat. The CRISPR repeat was followed by the Spacer 1 sequence ((Sp1, SEQ ID NO:82) and a partial CRISPR repeat consisting of the first 16 bp, followed directly by a terminator sequence (SEQ ID NO:85). The *CR-Sp* construct (SEQ ID NO:88) had a single processed form of the CRISPR repeat (SEQ ID NO:45 or SEQ ID NO:46) followed by a shortened 23 nt spacer sequence (SEQ ID NO:89) which was followed directly by a terminator sequence, that is, *CR-Sp* had the sequence of a processed guide inserted between the promoter and terminator of the construct.

As shown in FIGS. 17F and 17G, both NoMmc3 and BdMmc3, the minimal crRNA construct comprising the processed crRNA encoding sequence operably linked to the PJ23119 promoter (SEQ ID NO:84) supported equivalent activity to the un-processed CRISPR array plasmid (SEQ ID NO:86). These data support the predictions from the RNAseq data and define the processed crRNA as comprising an 18-19 bp RNA derived from the 3′ end of the CRISPR repeat that is upstream of RNA derived from the 5′ end of the spacer that need be no greater than 23 bp.

Additionally, the longer processed form (*CR-Sp-CR*), suggested by the BdMmc3 RNAseq, supported activity similar to that of the minimal construct described above. Based on these data, no additional functionality or relevance is ascribed to the longer processed form of the crRNA predicted from BdMmc3 RNAseq data.

### Example 5

### Multiplex Targeting with an Mmc3 System

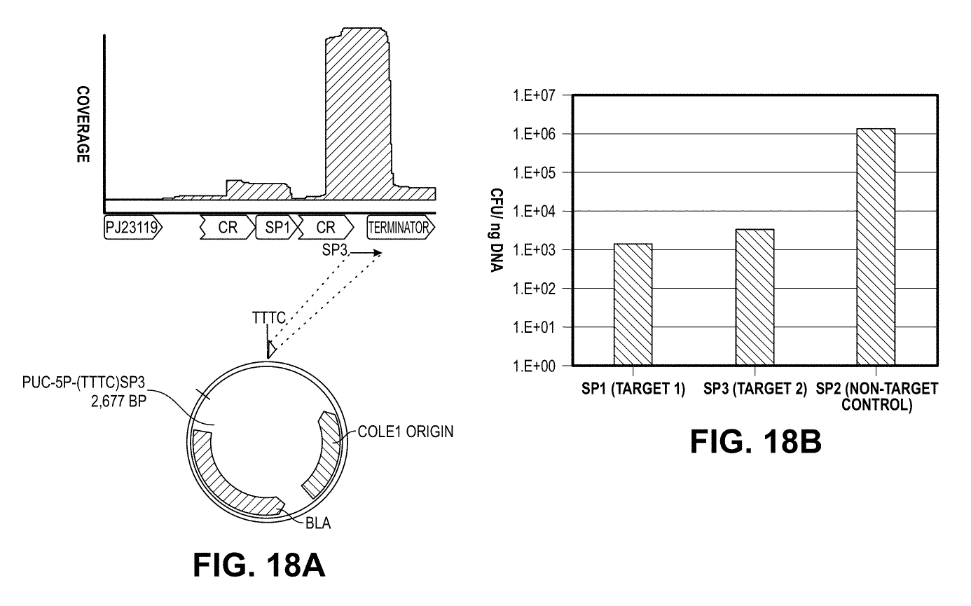

E. coli strains expressing a CRISPR array with two full repeat regions and an Mmc3 effector were observed to generate two processed crRNAs, one for each repeat (FIG. 18A). The first processed crRNA included the engineered spacer sequence (Sp1, SEQ ID NO:82) immediately 3′ to 18 bp of repeat sequence. The second processed crRNA, as determined by RNAseq, had a spacer sequence (Sp3; SEQ ID NO:90) derived from the terminator that followed the second repeat (see FIG. 18A). This was observed in all systems analyzed, for example, in the BdMmc3, NoMmc3, SfMmc3, and SvMmc3 systems. The ability of these effectors to process two different crRNAs from a single CRISPR array construct suggested the potential for targeting two protospacer sequences simultaneously using a single CRISPR array construct.

To test this hypothesis, a reporter plasmid was built with a spacer sequence compatible with the predicted terminator spacer sequence (Sp3, SEQ ID NO:90) and flanked by a 5′-TTTC PAM. Strains containing the full-length synthetic CRISPR array (repeat-spacer-repeat-terminator) were capable of targeting reporters containing either the Sp1 spacer (SEQ ID NO:82) or the terminator-derived spacer (Sp3, SEQ ID NO:90), as demonstrated in a plasmid interference assay. FIG. 18B provides the results of plasmid interference assays where the target plasmid included either the Sp1 or Sp3 spacer, where the number of colonies resulting from transformations that included a double spacer crRNA construct for targeting Sp1 (SEQ ID NO:82) and Sp3 (SEQ ID NO:90) were two to three orders of magnitude lower than colonies resulting from transformation with a reporter plasmid encoding non-targeting spacer Sp2 (SEQ ID NO:83). These results support applications of Mmc3 for multiplexed editing, as a single array was able to support multiple on-target DNA cleavage reactions.

### Example 6

### Genome Editing in E. coli with Mmc3

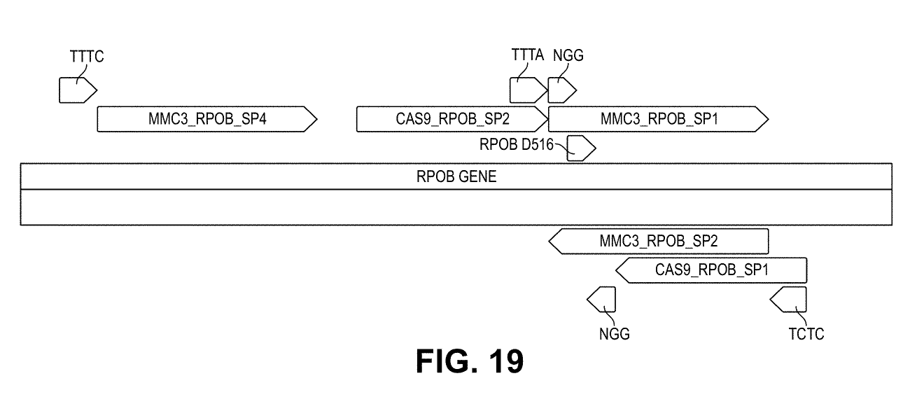

Mmc3 was tested for its ability to target a chromosomal locus in E. coli and facilitate repair-dependent editing. The genome target chosen was rpoB, the essential gene encoding RNA polymerase. Mmc3 effectors BdMmc3 (SEQ ID NO:1) and NoMmc3 (SEQ ID NO:6) were tested for their ability to target the rpoB locus using different crRNAs that included a 19 nt processed repeat sequence (5′-AATTTCTACTATTGTAGAT, SEQ ID NO:46) and different spacer sequences: Mmc3_rpoB_sp1 (SEQ ID NO:92), Mmc3_rpoB_sp2 (SEQ ID NO:93), and Mmc3_rpoB_sp4 (SEQ ID NO:94) (FIG. 19). S. pyogenes Cas9, a known Type II effector (“SpCas9”, SEQ ID NO:80) and Acidaminococcus sp. Cpf1, a known Type V effector (“AsCpf1”, SEQ ID NO:81) were assayed for comparison, where the AsCpf1 effector was tested using the Mmc3 guides, as the processed repeat sequences in Cpf1 editing systems differ from that of the Mmc3 processed repeat sequence by only a single nucleotide, and the Cas9 effector was tested using the Cas9-rpoB-Sp1 guide (SEQ ID NO:96, having guide (spacer) sequence SEQ ID NO:95) and Cas9-rpoB-sp2 guide (SEQ ID NO:98, having guide (spacer) sequence SEQ ID NO:97). E. coli does not possess NHEJ activity, therefore double-strand breaks in the chromosome cannot be repaired in the absence of a template for homology-dependent repair. The result of successful targeting by the Mmc3 effectors is therefore lack of viability.

Targeting of the chromosome instead of a plasmid therefore used a modified protocol since combining a plasmid for expressing an effector and a plasmid for expressing a CRISPR array (or crRNA) that targets the chromosome results in nonviable cells. Strains expressing the Mmc3 effector were transformed with target or non-target crRNA (control) plasmids followed by selection for maintenance of the crRNA plasmid. Because maintenance of a crRNA plasmid that supports chromosome cleavage would be lethal, the effective transformation rate is reduced when the effector and crRNA support chromosome cleavage relative to combinations of the effector and crRNA that do not support chromosomal cleavage.

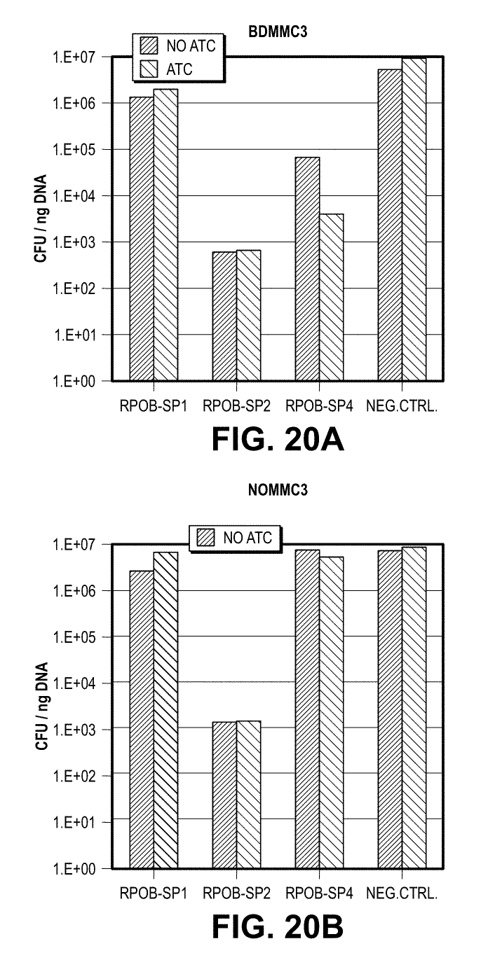

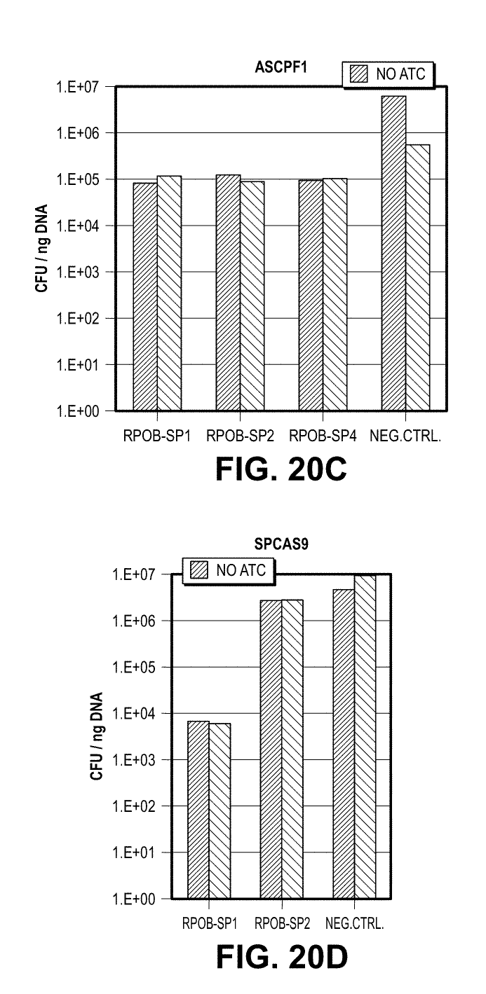

When tested in the manner outlined above, the effectors showed varied amounts of chromosome cleavage depending on the crRNA utilized (FIG. 20A-D). The BdMmc3 (SEQ ID NO:1) (FIG. 20A) and NoMmc3 (SEQ ID NO:6) (FIG. 20B) effectors demonstrated activity that was as good, if not better, than that of the Cas9 effector (SEQ ID NO:80) (FIG. 20D) for at least one crRNA (Mmc3_rpoB_sp2 (SEQ ID NO:100), >3 log reduction in transformants). Control effector AsCpf1 (SEQ ID NO:81) showed poor cleavage for all guides tested (FIG. 20C). While robust activity for both NoMmc3 and BdMmc3 was observed with the rpoB-Sp2 guide RNA (SEQ ID NO:100), the rpoB-Sp4 guide RNA (SEQ ID NO:101) showed robust cleavage for BdMmc3 only. The rpoB-Sp2 protospacer in the E. coli genome utilized a Mmc3-specific TCTC PAM providing independent support for the relaxed PAM requirements of Mmc3 effectors (see FIG. 14).

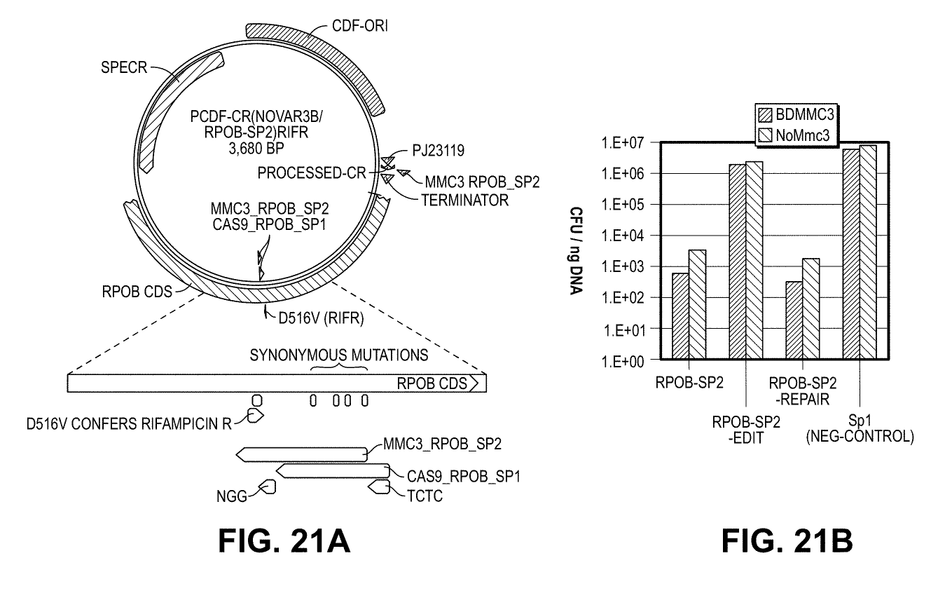

A number of point mutations in rpoB confer resistance to rifampicin, providing positive selection for mutant alleles. This allowed for testing Mmc3 effectors for the ability to facilitate mutation of the rpoB locus by homologous recombination with a repair or donor template. Introduction of the rpoB allele conferring rifampicin resistance (rifR) was achieved by cloning the repair template into the crRNA plasmid (FIG. 21A). The repair template (SEQ ID NO:102) included the D516V mutation with approximately 800 bp of rpoB gene homologous sequence on either side of the mutated site that confers rifampicin resistance. In addition, four synonymous mutations were introduced downstream of the D516V mutation to ablate the target site and prevent re-cleavage once the repair fragment had been integrated into the cleaved target site (FIG. 21A). To confirm the specificity of Mmc3-dependent rpoB cleavage, the same synonymous mutations from the repair template were introduced into the rpoB_sp2 crRNA plasmid as a control to confirm that the repair template was not recognized by the Mmc3 effectors. This ‘REPAIR’ guide sequence did not support chromosomal cleavage by BdMmc3 and NoMmc3 (FIG. 21B).

The protocol for testing CRISPR-assisted editing of the rpoB locus for rifR was as follows: E. coli NEB 10-beta was transformed with a rpoB crRNA plasmid that also included the repair template (SEQ ID NO:102). These strains were then transformed with either 1) the appropriate effector expression plasmid or 2) an empty vector control plasmid. Transformants were selected for on 1) LB+Cm to assess transformation frequency, 2) LB+Cm+Rif to assess the frequency of RifR (repair of rpoB locus) for transformed cells, 3) LB+Rif to assess population-wide rate of RifR, and 4) LB only to measure the number of viable cells. Calculation of the apparent frequency of RifR per transformed cell reports on the efficacy of CRISPR-assisted editing:

For experiments with crRNA+Repair & CRISPR effector

% editing=No. RifR-CmR transformants/No. CmR transformants*100%

For experiments with crRNA+Repair & no CRISPR effector

% recombination=No. RifR/No. viable cells*100%

For experiments with non-targeting crRNA (no Repair) & no CRISPR effector

% Spontaneous=No. RifR/No. viable cells*100%

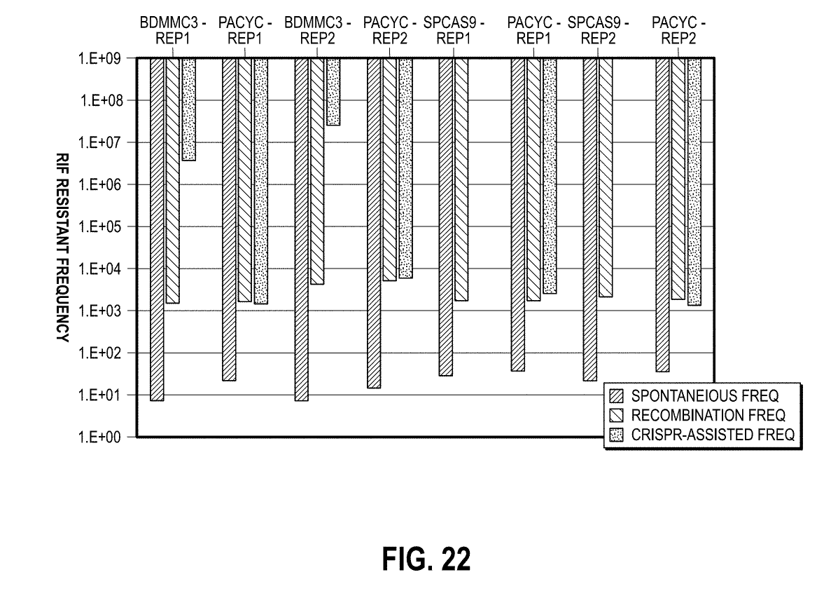

Using the above method, the efficacy of CRISPR-assisted editing for RifR was measured for BdMmc3 and SpCas9 (Table 10). Based on this analysis the BdMmc3 effector increased the apparent frequency of RifR between 3-4 orders of magnitude over the rate of recombination in the absence of BdMmc3 and approximately 6 orders of magnitude greater than the rate of spontaneous RIfR (FIG. 22). For the Cas9 effector expressed in a host that included a plasmid having a crArray encoding a Cas9-rpoB-sp2 guide in the donor plasmid, the frequency of RifR/transformed cells was below the limit of detection, preventing quantification of editing efficacy. As transformation frequencies for Cas9 and BdMmc3 effector plasmids were similar, BdMmc3 demonstrated greater efficacy for genome editing than SpCas9 in this system.

TABLE 10
CRISPR-mediated genome editing rates

| System | Variable | Rep1 | Rep2 |
|---|---|---|---|
| BdMmc3 | Effector (+) | 0.4% | 2.3% |
| | Effector (−) | 0.00013% | 0.000561% |
| | Fold-enrichment | 3019 | 4167 |
| Cas9 | Effector (+) | N/A | N/A |
| | Effector (−) | 0.00024% | 0.000125% |
| | Fold-enrichment | N/A | N/A |

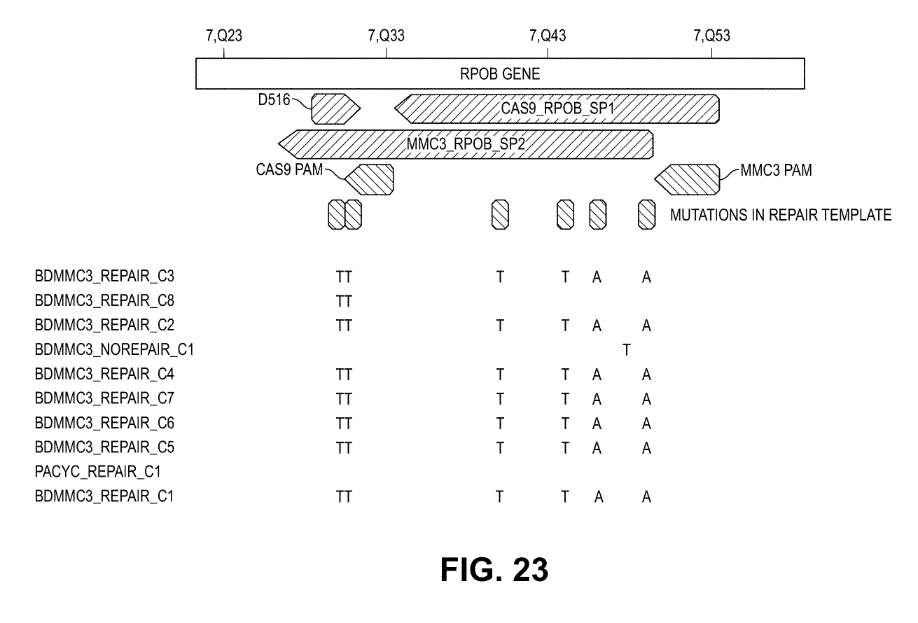

Sequencing of several RifR clones confirmed the presence of the D165V mutation and the synonymous mutations present in the repair template (FIG. 23). The wild type sequence was confirmed in RifR clones that did not have the repair template. Together these data support the conclusion that BdMmc3 was highly effective at mediating gene editing and repair with a template that allowed introduction of a D516V RifR allele and ablation of the BdMmc3 cleavage site.

### Example 7

### Mutation of Predicted RuvC Catalytic Residues and Zinc Finger Domain

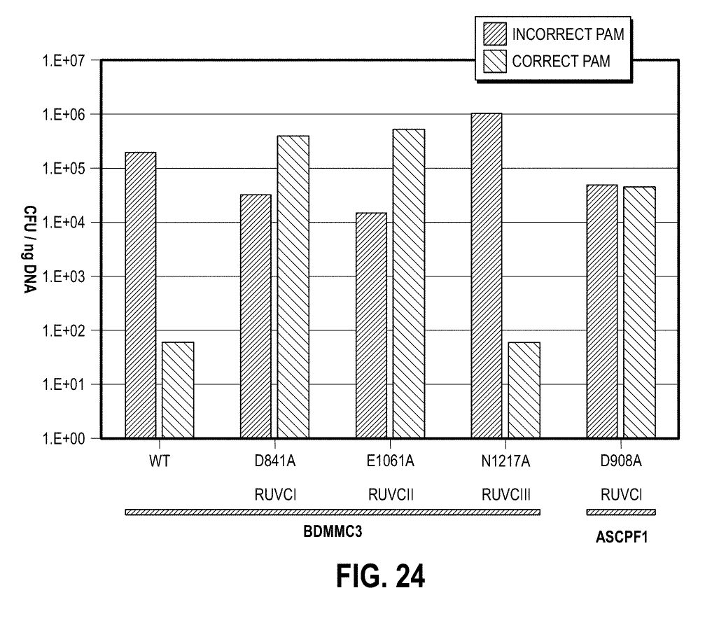

The RuvC-like domains of Mmc3 effectors contain the catalytic residues predicted to be responsible for the nuclease activity. To confirm these predictions, three mutants of BdMmc3 were constructed to replace each predicted catalytic residue with alanine. Mutants D841A, E1061A and N1217A were tested for nuclease activity using the standard plasmid interference assay in E. coli (see Example 2). Mutations D841A and E1061A completely abolished DNA cleavage, whereas mutation of the RuvC III domain (N1217A) did not affect DNA cleavage activity relative to the wild type effector control (FIG. 24). (Mutation of the RuvC III catalytic domain in Cpf1 also had little effect on DNA cleavage activity.) Together, these results support the bioinformatic analysis of the RuvC catalytic residues of Mmc3 effectors and confirms the identified RuvC II motif (FIGS. 5A and 5B) which was found to be uniquely positioned within the Mmc3 protein relative to its position in effectors of other known TypeV systems (Example 1).

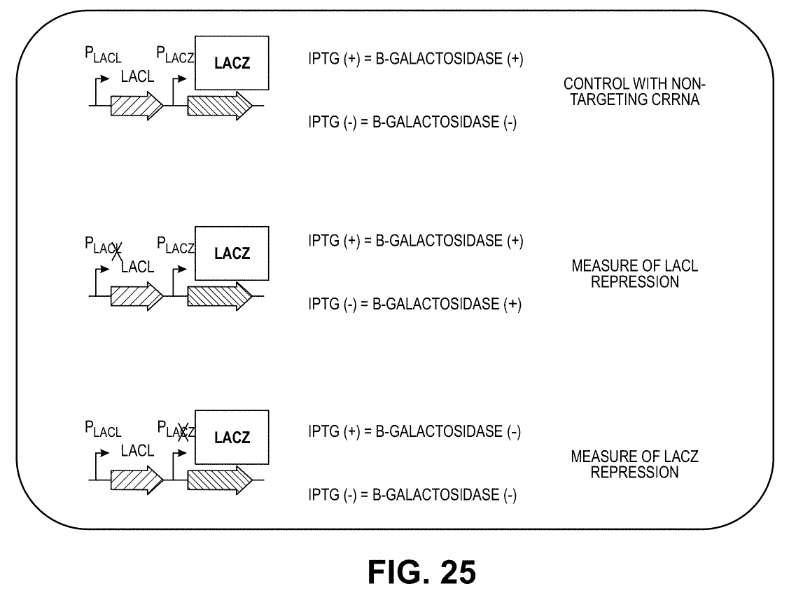

Mmc3 systems having effectors that included mutations designed to disable the nuclease function while retaining sequence-specific DNA binding (referred to herein as dMmc3 systems) were also tested in E. coli for their ability to bind dsDNA and thereby inhibit transcription of genes. The test system was composed of three parts: 1) a mutated Mmc3 effector gene cloned into a vector (pACYC) under control of an inducible P_{Tet} promoter; 2) a synthetic CRISPR array encoding two to three non-natural spacers expressed from a constitutive promoter on a medium copy number vector (pCDF, Kim, J. S. and Raines, R. T. (1993) Protein Science 2: 348-356) and 3) lacI and lacZ genes encoded in the chromosome of E. coli MG1655 strain (Table 11). When co-expressed in the same cell, specific association of the Mmc3 effector with a cognate crRNA directs the effector to bind double-stranded (ds) DNA at sequences containing a sequence complementary to the spacer sequence of the crRNA (the target site), where the target sequence occurs downstream of a TTTV motif (the PAM for the Mmc3 effectors). The binding of the dMmc3 effector to target sites within the lacI and lacZ genes blocks transcription of these genes. The inhibition of transcription of either LacI or LacZ can be measured by a photometric assay using ortho-Nitrophenyl-β-galactoside (ONPG) as the substrate for the LacZ enzyme. Repression of the lacI gene by dMmc3 can be measured by detecting an increase in β-galactosidase activity as a product of LacZ expression, while repression of the lacZ gene by dMmc3 can be measured by detecting a decrease in β-galactosidase activity in the presence of IPTG when compared to strains expressing a non-targeting crRNA (FIG. 25).

Using this system, DNA binding in vivo by the Mmc3 effector dBdMmc3 was tested and compared to transcriptional repression mediated by dAsCpf1. Effector genes were synthesized to encode effectors having a mutation that changes the aspartate at residue number 908 of AsCpf1 and at residue number 841 of BdMmc3 (within the RuvC I domain, FIG. 5) to alanine. The resulting mutant effectors are referred to as dAsCpf1 and dBdMmc3, respectively. The mutated effector genes were synthesized by PCR using primers that incorporated the mutated codons and cloned into the pACYC vector under the control of the P_{TET} promoter that is induced by the addition of tetracycline to the culture medium.

Assays to demonstrate binding of the dAsCpf1 and dBdMmc3 effectors to sites in the lacI and lacZ genes were performed by transforming E. coli strain MG1655 that included the lacI and lacZ genes integrated into the chromosome with a construct that included a gene encoding a mutant effector (dAsCpf1 or dBdMmc3) and a construct that encoded a crRNA array, where the crRNA included multiple units of cognate CRISPR repeat and spacer sequence. Two to three crRNA units were encoded in each array as set forth in Table 11.

TABLE 11
Lac I and LacZ Target Sequences

| CRarray / Target name | Target sequence |
|---|---|
| LacI_array_target 1 | CTCGAGTGCAAAACCTTTCGCGGTATGG |
| | (SEQ ID NO: 201) |
| LacI_array_target 2 | GCGGTATGGCATGATAGCGCCCGGAAGA |
| | (SEQ ID NO: 202) |
| LacI_array_target 3 | AATAGGCGTCGAGGCCTTTGCTCGAGTG |
| | (SEQ ID NO: 203) |
| LacZ_array A_Target 1 | CAACGTCGTGACTGGGAAAACCCTGGCG |
| | (SEQ ID NO: 204) |
| LacZ_array A_Target 2 | GCCAGCTGGCGTAATAGCGAAGAGGCCC |
| | (SEQ ID NO: 205) |
| LacZ_array A_Target 3 | ATGTTGATGAAAGCTGGCTACAGGAAGG |
| | (SEQ ID NO: 206) |
| LacZ_array B_Target 1 | CAACGTCGTGACTGGGAAAACCCTGGCG |
| | (SEQ ID NO: 207) |
| LacZ_array B_Target 2 | CTGTGTGAAATTGTTATCCGCTCACAAT |
| | (SEQ ID NO: 208) |

Freshly prepared competent cells (25 uL) of E. coli MG1655 were electroporated with 50 ng of a plasmid for expression of the mutant effector that included a chloramphenicol resistance gene and 50 ng of a plasmid for expression of the cognate crRNA as a control. Each of the dAsCpf1 and dBdMmc3 effectors was separately co-transformed with a construct having a spectinomycin resistance gene and encoding a cognate crRNA that included either a guide sequence targeting LacZ, a guide sequence targeting LacI, or a non-targeting guide sequence as a control. Electroporation was performed using 1 mM cuvettes and the standard Biorad electroporator settings for bacteria (1.8 kV, 200 mW, 25μF) Immediately after electroporation cells were re-suspended in 900 uL of SOC medium and incubated at 37° C. for 1 h. Transformations were plated on LB plates containing chloramphenicol (12.5 μg/mL) and spectinomycin (50 μg/mL). Plates were incubated overnight at 37° C. and colonies were in 5.0 mL of LB medium containing chloramphenicol (12.5 μg/mL) and spectinomycin (50 μg/mL) and incubated at 37° C. overnight. The next morning 25 μL of the overnight cultures were transferred to 2.5 mL of LB chloramphenicol (12.5 μg/mL), spectinomycin (50 μg/mL) and anhydrotetracycline (100 ng/mL) containing IPTG concentrations of 0 to 1 mM of IPTG and incubated at 37° C. until the cultures reached OD600 of 0.4-0.6. Cultures were normalized to a final OD600 of 0.8 and centrifuged at 5000×g for 5 min at 4° C. Supernatants were removed and pellets were resuspended in 1.0 mL of Lysis buffer (100 mM Tris/HCl, pH 7, 100 mM KCl, 10 mM MgCl₂, 35 mM DTT, 1 mg/mL lysozyme, 2.0 U/mL benzonase (Sigma E1014-25KU), 0.10% Triton X-100, 1 mg/mL ONPG (Sigma N1127). Plates were centrifuged at 5000×g for 5 min at 4° C. and supernatants were transferred to 96 well plates. Absorbance of supernatants were measured at 420 nm for quantification of relative expression of β-galactosidase activity. Microplate-reader β-galactosidase assays in E. coli were adapted from Schaefer et al. (2016) Analytical Biochemistry 503:56-57.

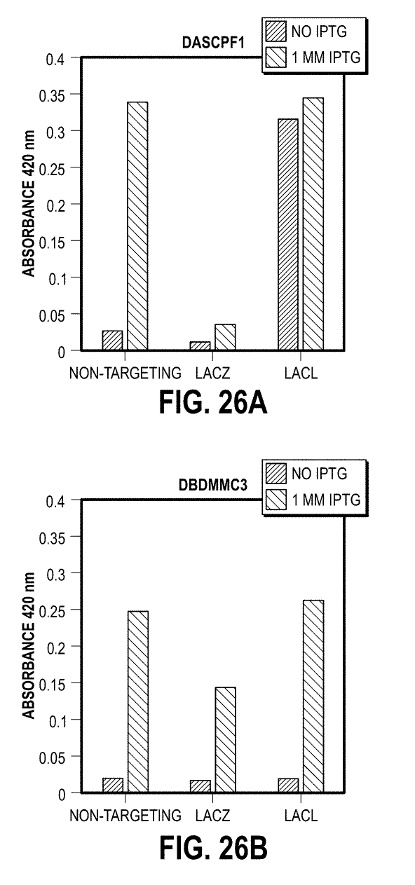

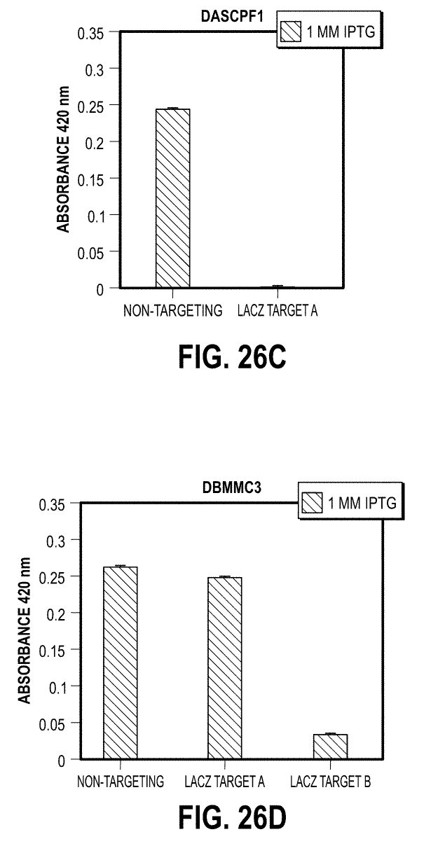

FIGS. 26A-D show the RNA-guided and DNA-interference activity for dAsCpf1 (D908A) and dBdMmc3 (D841A) when co-expressed with crRNAs targeting LacI and LacZ genes in E. coli as indicated by the reduction in absorbance at 420 nm from the cleaved β-galactosidase substrate. dAsCpf1 showed 9-fold repression of LacZ when tested with CRarray A that included three target sequences (FIG. 26A), while 0.4-fold repression was observed for dBdMmc3 using a CRarray against the same targets (FIG. 26B). For the Lad gene, dAsCpf1 showed 12-fold repression (FIG. 26A) while no repression was detected by dBdMmc3 (FIG. 26B). Additional targets within the LacZ gene were further tested with dBdMmc3 and showed that target B can yield up to 6.5-fold repression of LacZ (FIG. 26D), demonstrating that an Mmc3 effector mutated in a critical nuclease domain is able to bind the target site and affect transcription when the target site is within or upstream of a gene.

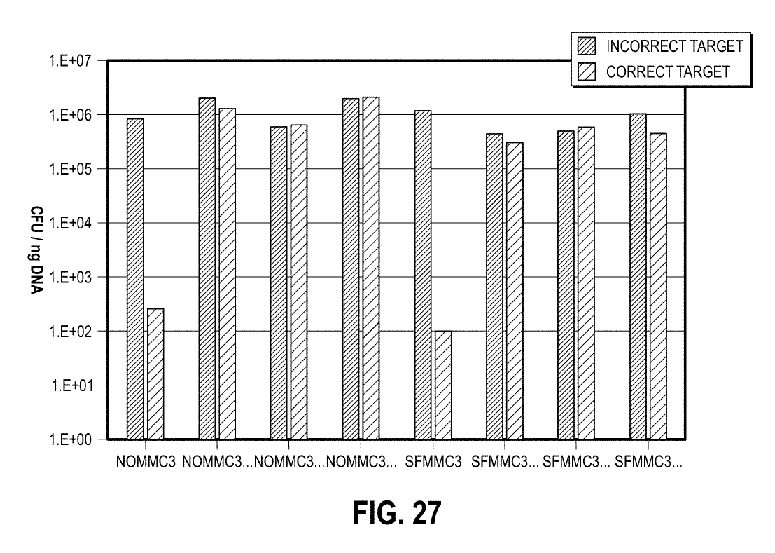

The functional significance of the zinc finger domain that is located between RuvC II and RuvC III motifs was also examined using the plasmid interference assay. The cysteine pairs of the NoMmc3 and SfMmc3 effectors were mutated to alanine as pairs and all together. For NoMmc3, the first cysteine pair mutations were C1123A and C1126A and the second cysteine pair mutations were C1138A and C1142A. The NoMmc3 having all four cysteines (both pairs) mutated had all of the C1123A, C1126A, C1138A, and C1142A mutations. For SfMmc3, C1205 and C1208 of the first cysteine pair and, independently, C1249 and C1252 of the second cysteine pair were mutated to alanine, and in an additional mutant, both SfMmc3 cysteine pairs (all four cysteine residues: C1205, C1208, C1249, and C1252) were mutated to alanine. FIG. 27 shows that the alanine mutations at either cysteine pair of the zinc finger domain completely abolish effector cleavage activity, highlighting the significance of this domain in Mmc3 effectors.

### Example 8

### Mmc3 Activity in Fungal Cells

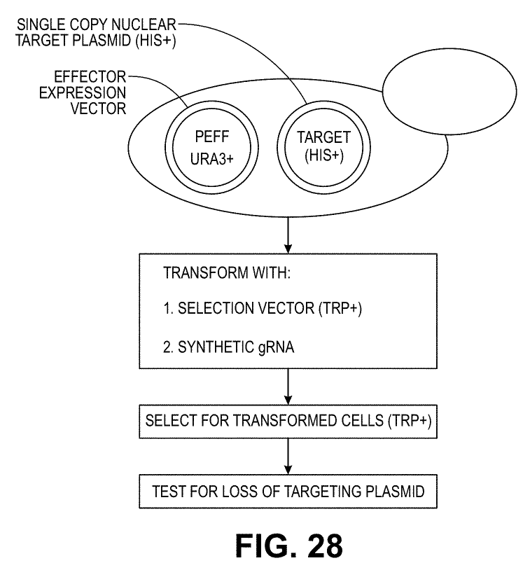

Mmc3 effectors were tested for their ability to cleave a nuclear localized plasmid in two yeast hosts, Saccharomyces cerevisiae and Kluyveromyces marxianus. The assays were designed such that cleavage of target plasmids in these hosts resulted in loss of the plasmids from the cell and an inability to grow in the absence of histidine due to loss of the linked his3 marker (FIG. 28).

In independent assays, a gene encoding the BdMmc3 effector codon-optimized for Saccharomyces cerevisiae (SEQ ID NO:103) and a gene encoding the NoMmc3 effector codon-optimized for S. cerevisiae (SEQ ID NO:104), as well as a S. cerevisiae codon-optimized gene encoding a SpCas9 effector (SEQ ID NO:105) and a S. cerevisiae codon-optimized gene encoding zn AsCpf1 (SEQ ID NO:106), were expressed constitutively from an extrachromosomal plasmid carrying a ura3 marker. The codon optimized Cas9, AsCpf1, BdMmc3 and NoMmc3 effector genes were constitutively expressed using the S. cerevisiae FBA1 promoter (SEQ ID NO:107). The AsCpf1, BdMmc3 and NoMmc3 effectors each carried a c-myc NLS and an SV40 NLS in tandem and followed by a peptide linker and 8× His tag (SEQ ID NO:108). The SpCas9 polypeptide included a C-terminal SV40 NLS (encoded by SEQ ID NO:109). The effector, targeting, and selection plasmids carried CEN/ARS sequences for propagation as single-copy plasmids in both S. cerevisiae and K. marxianus.

The target plasmid carrying the his3 marker was maintained in the strains together with the effector plasmids that constitutively expressed the effector proteins by growing cells in defined media lacking uracil and histidine. The yeast strains were made electrocompetent and transformed with three separate guide RNAs (gRNAs), each targeting a separate sequence of the OriT sequence of the plasmid (see Table 12). For each effector tested, a control transformation was carried out with a non-targeting gRNA (SEQ ID NO:110) in place of an actual target sequence. A selection plasmid carrying the trp1 marker was co-transformed alongside the gRNAs to select for transformed cells. The procedure for electroporation was essentially the same as described in Kannan et. al. (2016) Sci. Rep. 6, 30714; doi: 10.1038/srep30714.

During each transformation, 2 μg of the in vitro transcribed gRNAs (Table 11) and 200 ng of the selection plasmid were included. After electroporation using parameters 2.5 kV, 200Ω, 25 μF, cells were recovered overnight at 30° C. in 2 ml of non-selective recovery media (1:1 ratio of YPAD:1M Sorbitol). 100 μl of the recovered cells were plated on agar plates lacking uracil and tryptophan (selecting for the effector plasmid and cells that were transformed with the trp1 selection plasmid co-transformed with the gRNA) and incubated overnight at 30° C. Twenty-four to twenty-five colonies from each plate were patched onto agar plates lacking uracil and histidine and incubated overnight at 30° C. The number of colonies that did not produce growth when patched on plates lacking uracil and histidine (indicating the presence of the effector plasmid and the target plasmid, respectively) were recorded.

TABLE 12
Guide RNAs for DNA Editing

| | SEQ ID | |
|---|---|---|
| Guide RNA | NO | Description |
| Control Guide RNA used to test | 110 | Guide includes random target sequence, |
| Bd, No, Sh, Sf, Smp2 Mmc3 effectors | | does not target test nucleic acid molecule |
| Bd_OriT_T1 crRNA, used to test Bd, | 111 | 18 nt repeat followed by T1 spacer |
| No, Sh, Sf, & Smp2 Mmc3 effectors | | |
| Bd_OriT_T2 crRNA, used to testBd, | 112 | 18 nt repeat followed by T2 spacer |
| No, Sh, Sf, & Smp2 Mmc3 effectors | | |
| Bd_OriT_T3 crRNA, used to test Bd, | 113 | 18 nt repeat followed by T3 spacer |
| No, Sh, Sf, & Smp2 Mmc3 effectors | | |
| Control Guide RNA used to test | 114 | Guide includes random target sequence, |
| SfpMmc3 effector | | does not target test nucleic acid molecule |
| Sfp_OriT_T1 crRNA, used to test | 115 | 18 nt repeat followed by T1 spacer |
| Sfp Mmc3 effector | | |
| Sfp_OriT_T2 crRNA, used to test | 116 | 18 nt repeat followed by T2 spacer |
| SfpMmc3 effector | | |
| Sfp_OriT_T3 crRNA, used to test | 117 | 18 nt repeat followed by T3 spacer |
| SfpMmc3 effector | | |
| Control Guide RNA used to test | 118 | Guide includes random target sequence, |
| AsCpf1 effector | | does not target test nucleic acid molecule |
| AsCpf1_OriT_T1 crRNA | 119 | 20 nt repeat followed by T1 spacer |
| AsCpf1_OriT_T2 crRNA | 120 | 20 nt repeat followed by T2 spacer |
| AsCpf1_OriT_T3 crRNA | 121 | 20 nt repeat followed by T3 spacer |
| Control Guide RNA used to test | 122 | Guide includes random target sequence, |
| Cas9 effector | | does not target test nucleic acid molecule |
| Cas9_OriT_T1 guide RNA | 123 | Cas9 chimeric guide with T1 guide |
| | | sequence |
| Cas9_OriT_T2 guide RNA | 124 | Cas9 chimeric guide with T2 guide |
| | | sequence |
| Cas9_OriT_T3 guide RNA | 125 | Cas9 chimeric guide with T3 guide |
| | | sequence |

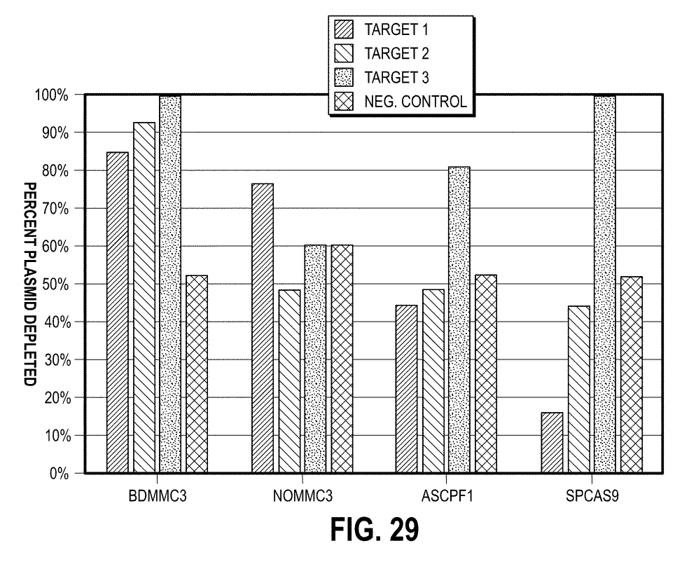

Initially, the Cas9, AsCpf1, BdMmc3, and NoMmc3 effectors were tested in K. marxianus, with three on-target gRNAs and one random non-targeting gRNA (‘Neg. Control’ in FIG. 29) tested for each effector strain (see Table 12). Plasmid depletion above background was observed with at least one of the three on-target guides for each of the Cas9, AsCpf1 and NoMmc3 expressing strains. The BdMmc3-expressing strain showed plasmid depletion above background for all three on-target guides (FIG. 29, Tables 13 and 14).

In this assay system, background plasmid depletion is defined by the frequency of plasmid loss that occurs in the absence of CRISPR mediated cleavage. For K. marxianus this level is approximately 50% of clones. There are two ways to normalized for this background: 1) assume background is not independent of active depletion of the plasmid and subtract the background from the experimental measurement and divide by the number of colonies screened, or 2) assume background is independent from active depletion of the plasmid—subtract the background from the experimental measurement and divide by the number of colonies screened adjusted for the expected frequency of non-specific plasmid loss. Method 1 gives a more conservative estimate than method 2. The editing percentages for BdMmc3 using normalization method 1 were for target 1, 32%; for target 2, 40%; and for target 3, 48%. The editing percentages for BdMmc3 using normalization method 2 were target 1, 67%; target 2, 83%; and target 3, 100% (Tables 13 and 14).

This initial experiment was replicated with BdMmc3 alone and yielded similar results: plasmid depletion indicated effector activity was well above background for all three target guides (Tables 15 and 16).

Cas9 and BdMmc3 effectors were also assessed in S. cerevisiae for their ability to cure the nuclear plasmid in a similar manner to that described for K. marxianus. The rate of plasmid loss with the randomized (Neg. Control) guide RNA was much lower for S. cerevisiae suggesting that the target plasmid is intrinsically more stable in S. cerevisiae. Both Cas9 and BdMmc3 expressing strains demonstrated plasmid depletion above background for all three on-target guides. The efficiency of plasmid cleavage and depletion was high for all three guides. The editing percentages for BdMmc3 using normalization method 1 were target 1, 84%; target 2, 84%; and target 3, 84%. The editing percentages for BdMmc3 using normalization method 2 were target 1, 100%; target 2, 100%; and target 3, 100% (Tables 17 and 18). The editing percentages for Cas9 using normalization method 1: target 1, 76%; target 2, 72%; and target 3, 72%. Editing percentages for Cas9 using normalization method 2 were target 1, 95%; target 2, 89%; and target 3, 89% (Tables 16 and 17).

The initial experiment in S. cerevisiae was replicated with BdMcm3 and Cas9, as well as with additional CRISPR effectors NoMmc3 and AsCpf1. BdMmc3 and Cas9 again showed high activity, with all cells examined depleted for the target plasmid. The editing percentages for BdMmc3 using normalization method 1 were target 1, 72%; target 2, 72%; and target 3, 72%. The editing percentages for BdMmc3 using normalization method 2 were target 1, 100%; target 2, 100%; and target 3, 100% (Tables 19 and 20). The editing percentages for Cas9 using normalization method 1: target 1, 76%; target 2, 76%; and target 3, 76%. Editing percentages for Cas9 using normalization method 2 were target 1, 100%; target 2, 100%; and target 3, 100% (Tables 19 and 20).

Additional codon-optimized Mmc3 CRISPR effector genes SfMmc3 (SEQ ID NO:126), ShMmc3 (SEQ ID NO:127), Smp2Mmc3 (SEQ ID NO:128), and SfpMmc3 (SEQ ID NO:129) were also tested in S. cerevisiae for editing capacity. These experiments utilized crRNAs with 18 nucleotide (nt) and 19 nt processed repeat sequences followed by a target sequence 3′ of the repeat sequence. Table 12 provides the SEQ ID NOs of guides having 18 nt repeat sequences. Guides that were tested for the SfMmc3, ShMmc3, SmpMmc3, and Smp2Mmc3 effector systems (SEQ ID NOs:110-113) that had an 18 nt “processed” repeat sequence had the processed repeat sequence of SEQ ID NO:45, and guides for the SfpMmc3 system that was tested (SEQ ID NOs:114-118) that had an 18 nt “processed” repeat sequence had the processed repeat sequence of SEQ ID NO:47. Guides having 19 nt repeat sequences had one additional nucleotide of the native repeat sequence at the 5′ end. For the SfMmc3, ShMmc3, SmpMmc3, and Smp2Mmc3 effector systems that were tested, the 19 nt guide RNAs included an additional ‘A’ at the 5′ end of the repeat sequence (SEQ ID NO:46). The 19 nt guide RNAs of the SfpMmc3 effector system that was tested also included an additional ‘A’ at the 5′ end of the repeat sequence (SEQ ID NO:48). Plasmid depletion above background was observed for Smp2Mmc3, SfMmc3 and SfpMmc3 effectors for at least one of the three on-target guides across experiments using 18 nt and 19 nt processed repeat sequences (Tables 21-24). In general, 19 nt repeat sequences resulted in a higher percentage of editing than 18 nt repeat sequences.

For experiments using an 18 nt processed repeat sequence, the editing percentages for SfMmc3 using normalization method 1 were target 1, 0%; target 2, 12%; and target 3, 16%. The editing percentages for SfMmc3 using normalization method 2 were target 1, 0%; target 2, 16%; and target 3, 21% (Tables 21 and 22). The editing percentages for SfpMmc3 normalization method 1 were target 1, 12%; target 2, 24%; and target 3, 8%. The editing percentages for SfpMmc3 using normalization method 2 were target 1, 16%; target 2, 32%; and target 3, 11% (Tables 20 and 21).

For experiments using a 19 nt processed repeat sequence, the editing percentages for SfMmc3 using normalization method 1 were target 1, 20%; target 2, 20%; and target 3, 0%. The editing percentages for SfMmc3 using normalization method 2 were target 1, 26%; target 2, 26%; and target 3, 0% (Tables 23 and 24). The editing percentages for SfpMmc3 normalization method 1 were target 1, 16%; target 2, 36%; and target 3, 32%. The editing percentages for SfpMmc3 using normalization method 2 were target 1, 21%; target 2, 47%; and target 3, 42% (Tables 23 and 24).

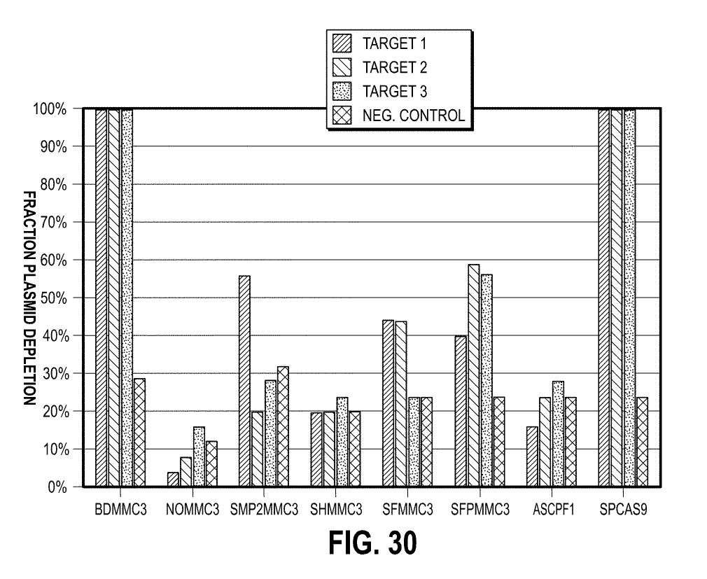

FIG. 30 shows a representative set of data for plasmid depletion experiments performed in S. cerevisiae.

TABLE 13
Normalized editing (%) for K. marxianus Rep1
(Assuming non-independence of experiment and control)

| Effector | Target 1 | Target 2 | Target 3 | Random |
|---|---|---|---|---|
| AsCpf1 | 0% | 0% | 28% | 0% |
| BdMmc3 | 32% | 40% | 48% | 0% |
| NoMmc3 | 16% | 0% | 0% | 0% |
| Cas9 | 0% | 0% | 48% | 0% |

TABLE 14
Normalized editing (%) for K. marxianus Rep1
(Assuming independence of experiment and control)

| Effector | Target 1 | Target 2 | Target 3 | Random |
|---|---|---|---|---|
| AsCpf1 | 0% | 0% | 58% | 0% |
| BdMmc3 | 67% | 83% | 100% | 0% |
| NoMmc3 | 40% | 0% | 0% | 0% |
| Cas9 | 0% | 0% | 100% | 0% |

TABLE 15
Normalized Editing (%) for K. marxianus Rep2
(Assuming non-independence of experiment and control)

| Effector | Target 1 | Target 2 | Target 3 | Random |
|---|---|---|---|---|
| BdMmc3 | 44% | 36% | 40% | 0% |

TABLE 16
Normalized Editing (%) for K. marxianus Rep2
(Assuming independence of experiment and control)

| Effector | Target 1 | Target 2 | Target 3 | Random |
|---|---|---|---|---|
| BdMmc3 | 65% | 53% | 59% | 0% |

TABLE 17
Normalized Editing (%) for S. cerevisiae Rep1
(Assuming non-independence of experiment and control)

| Effector | Target 1 | Target 2 | Target 3 | Random |
|---|---|---|---|---|
| BdMmc3 | 84% | 84% | 84% | 0% |
| Cas9 | 76% | 72% | 72% | 0% |

TABLE 18
Normalized Editing (%) for S. cerevisiae Rep1
(Assuming independence of experiment and control)

| Effector | Target 1 | Target 2 | Target 3 | Random |
|---|---|---|---|---|
| BdMmc3 | 100% | 100% | 100% | 0% |
| Cas9 | 95% | 89% | 89% | 0% |

TABLE 19
Normalized Editing (%) for S. cerevisiae Rep2
(Assuming non-independence of experiment and control)

| Effector | Target 1 | Target 2 | Target 3 | Random |
|---|---|---|---|---|
| AsCpf1 | 0% | 0% | 4% | 0% |
| BdMmc3 | 72% | 72% | 72% | 0% |
| NoMmc3 | 0% | 0% | 4% | 0% |
| Cas9 | 76% | 76% | 76% | 0% |

TABLE 20
Normalized Editing (%) for S. cerevisiae Rep2
(Assuming independence of experiment and control)

| Effector | Target 1 | Target 2 | Target 3 | Random |
|---|---|---|---|---|
| AsCpf1 | 0% | 0% | 5% | 0% |
| BdMmc3 | 100% | 100% | 100% | 0% |
| NoMmc3 | 0% | 0% | 5% | 0% |
| Cas9 | 100% | 100% | 100% | 0% |

TABLE 21
Normalized Editing (%) for S. cerevisiae Rep1 using 18 bp
processed repeat sequence (Assuming non-independence
of experiment and control)

| Effector | Target 1 | Target 2 | Target 3 | Random |
|---|---|---|---|---|
| Smp2Mmc3 | 0% | 0% | 0% | 0% |
| ShMmc3 | 0% | 0% | 0% | 0% |
| SfMmc3 | 0% | 12% | 16% | 0% |
| SfpMmc3 | 12% | 24% | 8% | 0% |

TABLE 22
Normalized Editing (%) for S. cerevisiae Rep1 using 18 bp
processed repeat sequence (Assuming independence of
experiment and control)

| Effector | Target 1 | Target 2 | Target 3 | Random |
|---|---|---|---|---|
| Smp2Mmc3 | 0% | 0% | 0% | 0% |
| ShMmc3 | 0% | 0% | 0% | 0% |
| SfMmc3 | 0% | 16% | 21% | 0% |
| SfpMmc3 | 16% | 32% | 11% | 0% |

TABLE 23
Normalized Editing (%) for S. cerevisiae Rep1 using 19 bp
processed repeat sequence (Assuming non-independence
of experiment and control)

| Effector | Target 1 | Target 2 | Target 3 | Random |
|---|---|---|---|---|
| Smp2Mmc3 | 24% | 0% | 0% | 0% |
| ShMmc3 | 0% | 0% | 4% | 0% |
| SfMmc3 | 20% | 20% | 0% | 0% |
| SfpMmc3 | 16% | 36% | 32% | 0% |

TABLE 24
Normalized Editing (%) for S. cerevisiae Rep1 using 19 bp
processed repeat sequence (Assuming non-independence
of experiment and control)

| Effector | Target 1 | Target 2 | Target 3 | Random |
|---|---|---|---|---|
| Smp2Mmc3 | 0% | 0% | 0% | 0% |
| ShMmc3 | 0% | 0% | 5% | 0% |
| SfMmc3 | 26% | 26% | 0% | 0% |
| SfpMmc3 | 21% | 47% | 42% | 0% |

### Example 9

### High Efficiency Mmc3 Chromosomal Editing in S. cerevisiae

To test the BdMmc3 effector for the ability to cleave and edit a chromosomal locus, the oriT region (SEQ ID NO:130) from plasmid pCC1BAC HIS3Km OriT was inserted into the chromosome of S. cerevisiae at the YAL044W-A locus. This oriT region was the same region targeted in plasmid depletion assays (Example 8) and allows use of the same validated crRNAs for chromosomal editing.

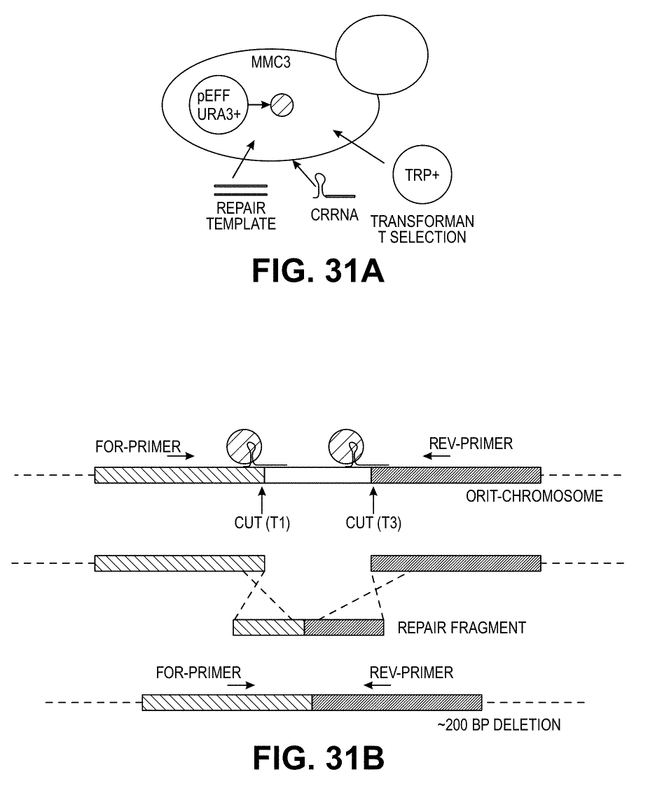

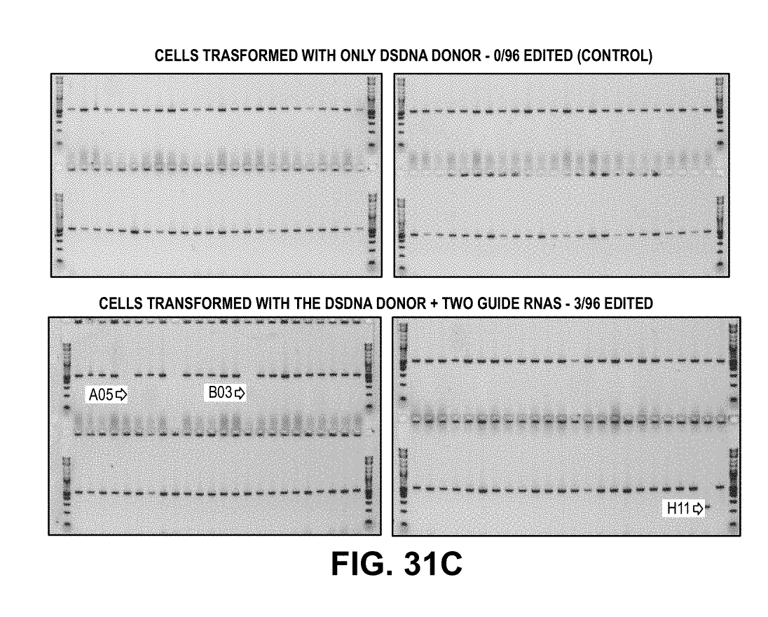

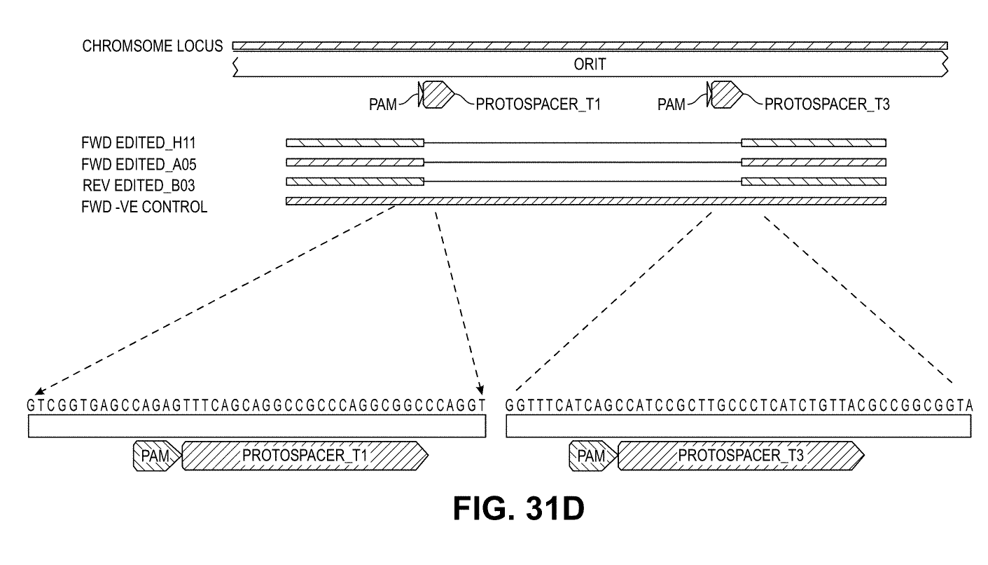

The codon-optimized BdMmc3 effector gene (SEQ ID NO:103) was expressed constitutively from an extrachromosomal plasmid that carried a ura3 marker, maintained by growth of the yeast cells in defined media lacking uracil. Strains were made electrocompetent and transformed with in vitro transcribed crRNAs targeting protospacers T1 (SEQ ID:131) & T3 (SEQ ID:132) in the OriT region (see Table 12) as well as a dsDNA repair fragment (SEQ ID:133) designed to introduce an approximately 250 bp deletion at the targeted locus by homologous recombination (FIG. 31A and 31B). For each effector tested, a control transformation was carried out with a non-targeting (non-cognate) crRNA. For all transformations, a plasmid carrying the trp1 marker was co-transformed with the in vitro transcribed crRNA and repair fragment to select for transformed cells. The procedure for electroporation was the same as described in Example 8, above, and Kannan et. al. (2016). During each transformation, 2 μg of the in vitro transcribed crRNA (Table 12), 200 ng of the selection plasmid and 10 pmoles of the dsDNA repair fragment were included. After electroporation, cells were recovered overnight at 30° C. in 2 ml of recovery media (1:1 ratio of YPAD:1M Sorbitol) prior to plating on selective media. After transformation, cells were allowed to recover overnight in non-selective media and then plated on agar plates with defined nutrients lacking uracil and tryptophan. Individual colonies were screened by PCR for the presence of the predicted deletion. Primers used were 1730 (SEQ ID:134) and 1731 (SEQ ID:135). The unedited wild type sequence gave a product of 487 bp, whereas the edited sequence gave a product of 243 bp.

As can be seen from FIG. 31C, BdMmc3 paired with T1 and T3 crRNA yielded 3 out of 96 clones with PCR products that were approximately 250 bp smaller than the wild type fragment, indicative of effector-mediated editing. Importantly, no editing was observed when the non-cognate guide was used, indicating that editing was not due to spontaneous homologous recombination of the donor at the OriT locus in the absence of BdMmc3-dependent cutting at T1 and T3 targets. Sequence analysis of the three positive clones and one negative clone gave the anticipated results with deletions observed between guide T1 and T3 for the three positive clones and no deletion present in the negative control (FIG. 31D).

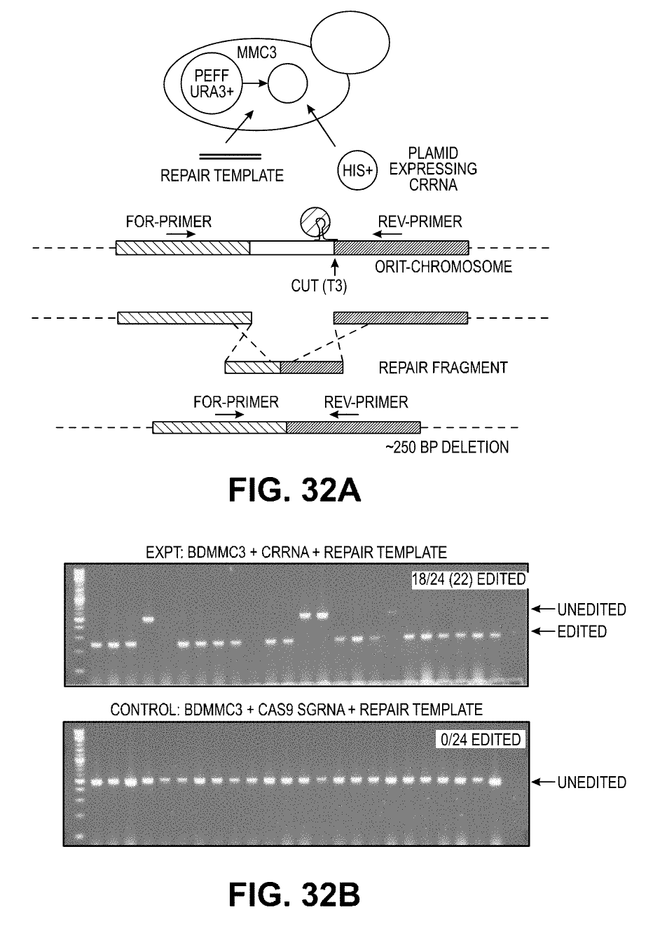

The experiment above utilized in vitro synthesized guides to target the OriT region cloned onto the chromosome. In a second experiment, editing was repeated using a plasmid to express the crRNA in vivo. For this experiment, yeast cells constitutively expressing BdMmc3 were transformed with a repair fragment as described above as well as a multicopy His-marked plasmid (pKD1322) encoding a minimal crRNA array composed of an SNR52 promoter (SEQ ID NO:136), a BdMmc3 full CRISPR repeat (SEQ ID NO:28), a spacer sequence targeting the oriT-T3 protospacer (SEQ ID NO:132), a second BdMmc3 CRISPR repeat and a SUP4 terminator (SEQ ID:138) (FIG. 32A). Transformants were selected on uracil and his dropout media prior to screening by colony PCR for the presence of the deletion in the oriT region. This analysis showed that 18/22 clones that produced an amplicon in the diagnostic PCR gave a product size consistent with deletion at the T3 target site, giving an editing efficiency of 82% (FIG. 32B). Again, no edited clones were observed when a non-cognate crRNA was used to target BdMmc3, reinforcing that the rate of spontaneous homologous recombination in the presence of a repair fragment is not sufficient to account for the frequency of editing we observe with BdMmc3 when paired with the correct crRNA.

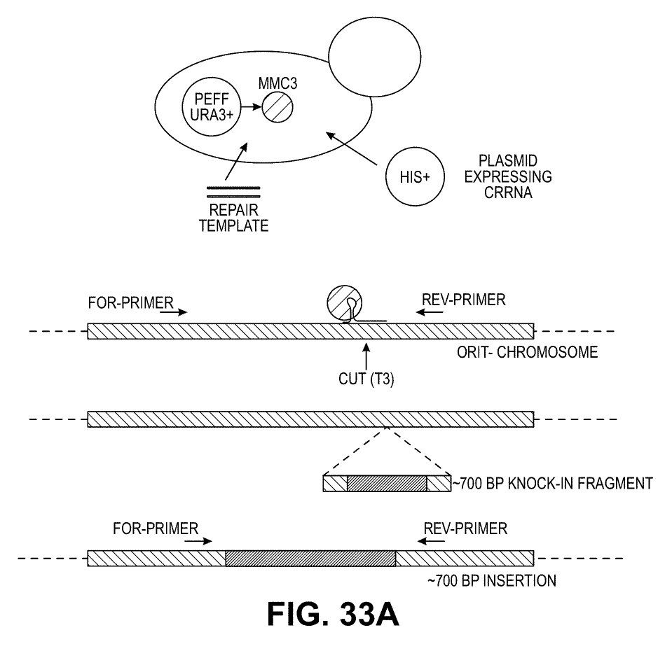

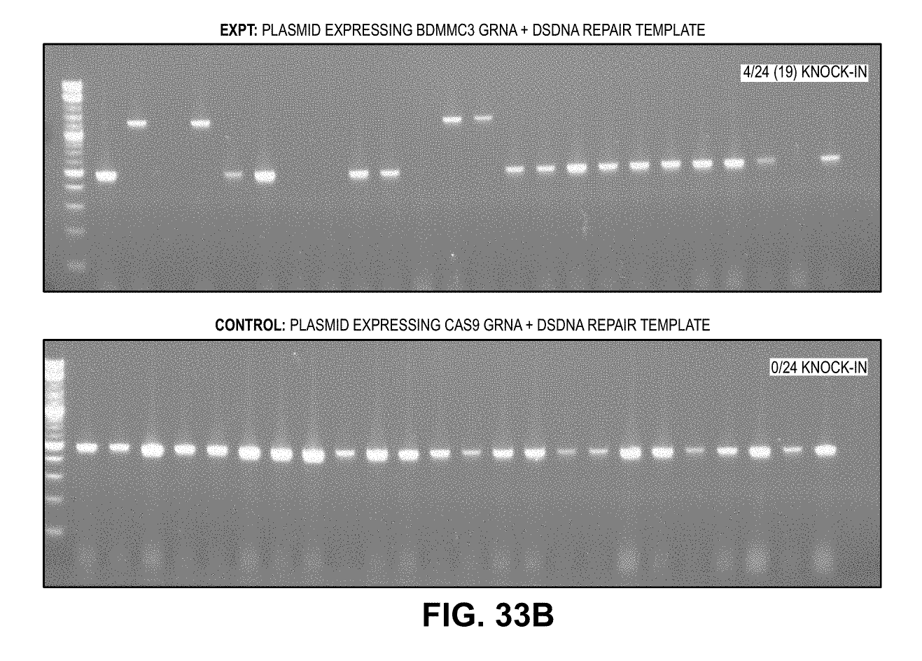

A third experiment was performed, following the same format as immediately above (the BdMmc3 effector expressed from an extrachromosomal plasmid and a minimal crRNA array targeting the oriT-T3 protospacer (SEQ ID NO:132) expressed from a different plasmid), except that a repair fragment was supplied that encoded an insertion of approximately 700 bp (SEQ ID NO:139) rather than a deletion (FIG. 33A). The repair fragment carried 40 bp homology arms. Colony PCR indicated correct insertion of approximately 700 bp fragment at the T3 locus for 4/19 amplified clones, giving a knock-in efficiency of 21% (FIG. 33B).

### Example 10

### Transfection of Mammalian K562 Cells with Mmc3 Constructs

Mmc3 effector genes were codon optimized for expression in mammalian cells and cloned into a vector under the control of the CMV promoter (SEQ ID NO:141) for constitutive expression. Codon optimized Mmc3 effector genes included the BdMmc3 effector gene (SEQ ID NO:143), NoMmc3 effector gene (SEQ ID NO:140), and the SfMmc3 effector gene (SEQ ID NO:145). Also included was a human codon-optimized gene encoding the AsCpf1 effector (SEQ ID NO:180) and a human codon-optimized gene encoding the Smp2Cpf1 effector (SEQ ID NO:142). The engineered Mmc3 genes and Cpf1 genes were designed as C-terminal translational fusions with GFP to allow monitoring of transfection and Mmc3 effector expression. The Mmc3 effector-encoding portion of the fusion gene was joined to the GFP-encoding portion of the fusion gene by a sequence encoding a self-cleaving 2a peptide (SEQ ID NO:147). The Mmc3 effector-encoding portion of the translational fusion gene also included sequences (SEQ ID NO:91) encoding an amino acid sequence that included a nucleoplasmin NLS from Xenopus, followed by a GS peptide linker and then a 3× HA tag (SEQ ID NO:148) immediately upstream of the 2a peptide and GFP encoding sequences.

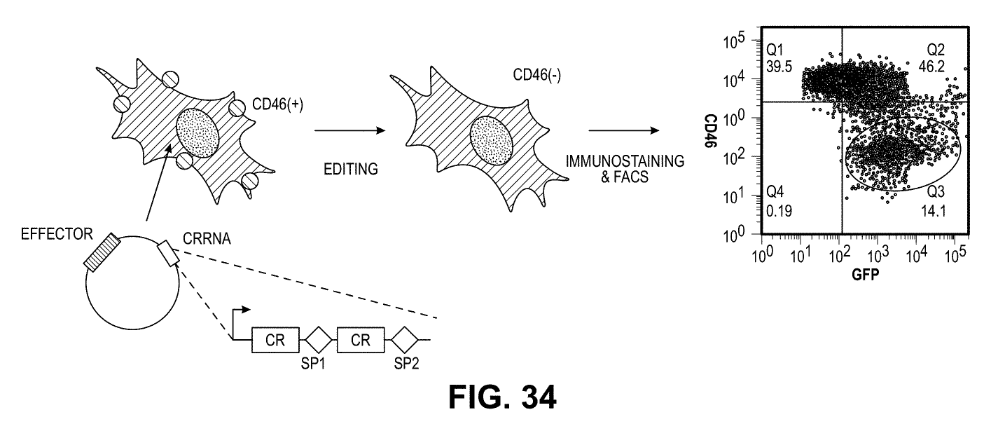

For each plasmid carrying a GFP-Mmc3 effector gene, a two-spacer multiplexed crRNA cassette specific to the Mmc3 type was cloned into the MauBI site in the plasmid backbone (FIG. 34). The crRNA cassette included the Human U6 promoter (SEQ ID NO:149) followed by the Mmc3 or Cpf1 CRISPR repeat (e.g., SEQ ID NO:28 for BdMmc3, SEQ ID NO:33 for Smp2Cpf1, SEQ ID NO:40 for NoMmc3, SEQ ID NO:29 for SfMmc3), a spacer sequence targeting CD46_exon1 (CD46_540sp, (SEQ ID NO:150), another copy of the Mmc3 CRISPR repeat, and a second spacer sequence CD46_541sp (SEQ ID NO:151) targeting a second location on the CD46_exon1 (SEQ ID NO:152). The crRNA cassettes ended in a polyT tract for transcript termination (SEQ ID NO:153). The NoMmc3 crRNA expression cassette used in the plasmid carrying the Smp2Cpf1 effector gene (SEQ ID NO:142) is provided as SEQ ID NO:154; the BdMmc3 crRNA expression cassette used in the plasmid carrying the BdMmc3 effector gene is provided as SEQ ID NO:155; the Smp2Mmc3 crRNA expression cassette used in the plasmid carrying the NoMmc3 effector gene is provided as SEQ ID NO:156; and the SfMmc3 crRNA expression cassette used in the plasmid carrying the SfMmc3 effector gene is provided as SEQ ID NO:157. The AsCpf1 crRNA expression cassette used in the plasmid carrying the AsCpf1 effector gene is provided as SEQ ID NO:190.

CD46 is a cell surface marker that can be detected with fluorescently labeled antibodies (e.g., a monoclonal antibody (MEM-258, Thermo Fisher) labeled with APC (Life Technologies, A15711). Loss of this marker by mutation of the coding region and subsequent dilution of receptors expressed on the cell surface by growth can be detected by flow cytometry. FIG. 34 shows, on the right, an example of a scatter plot in which each dot represents a single cell quantitated by flow cytometry for fluorescence from CD46 antibody staining on the Y axis, and for GFP fluorescence on the X axis. The horizontal line within the graph marks the fluorescence threshold that captures greater than 99% of CD46 detected on the surface of non-transformed cells above background, and the vertical line within the graph marks the cutoff value for fluorescence above background for GFP. Dots in the lower right quadrant therefor represent cells demonstrating GFP expression (and, because the GFP gene transformed into the cells is a translational fusion with the Mmc3 effector gene, also expressing the Mmc3 effector) and having reduced CD46 staining with respect to nontransformed cells.

To transfect K562 cells with plasmids expressing an Mmc3 effector and multiplexed crRNA targeting CD46, K562 (ATCC CCL-243) cells were grown in RPMI 1640 Medium, GlutaMAX Supplement (LifeTech 61870127) plus 10% FBS (Clontech 631367) and passaged every other day. Passaging of cells was done by diluting the culture to 0.3×10⁶ cells per ml and then plating 13 ml in a T75 flask. At the time of splitting of the culture there was usually between 0.7 and 1.3×10⁶ cells per ml. (Cells were kept in culture for less than a month before a new vial was thawed). Cells were nucleofected at the time they would normally be split. Cells were nucleofected using SF Cell Line 96-well Nucleofector™ Kit (96 RCT) (Lonza V4SC-2096) following the 4D protocol: Cells were counted and centrifuged for 10 minutes at 90×g. The approximately 200,000 cells were then resuspended in 16.4 μl SF buffer with 3.6 μl supplement then 20 μl was aliquoted into sterile pipet tubes. Up to 2 μl of DNA (2-4 μg) was added and mixed gently before transferring to nucleocuvette strips. The cells were shocked with a 4-D Nucleofector™ Core Unit electroporator (Lonza, AAF-1002B) with attached 4-D Nucleofector™ X Unit (Lonza, AAF-1002B) using program FF-120 and allowed to rest 10 minutes at room temperature before adding 100 μl of media pre-warmed to 37° C. The cells were transferred to 24 μl plates containing 400 μl of warm media, and cultures were split two days later.

Analysis of Mammalian K562 Cells Transformed with Mmc3 Effectors and Guide RNAs

To prepare cells for flow cytometry, two to four days after transfection cells are spun down and resuspend in 50 μl FACs buffer and 2 μl CD46 Monoclonal Antibody (MEM-258), APC (Lifetech A15711) for detection of the CD46 cell surface marker. Cells are stained 20-30 min in PBS+2% FBS+0.2% sodium azide at 4° C., washed with 750 μl buffer and resuspended in 200-400 μl buffer for analysis.

Samples are analyzed on a ZE5 flow cytometer (BioRad). GFP is excited at 488 nm and emission spectra is detected with a 525/35 nm bandpass filter. CD46 antibody conjugated to allophycocyanin (APC) is excited at 640 nm and emission spectra is detected with a 670/30 bandpass filter. The flow rate is 1000-2000 events/second. Forward and side scatter signals are used to define and gate total cells. Greater than 20,000 events are recorded for each sample and analyzed using FlowJo software (flowjo.com/) for the presence of CD46 staining in GFP-expressing cells. Cultures in which loss of CD46 expression on the cell surface of GFP-expressing cells is observed by loss of APC fluorescence with respect to controls can be analyzed by sequencing of the CD46 locus for indels associated with disruption of the CD46 gene (see FIG. 34).

For genomic DNA analysis, an aliquot of cells is spun down, the media removed, and the cells are lysed in QuickExtract™ DNA Extraction Solution (Epicentre QE09050) at about 20,000 cells/μl solution at 65° C. for 6 minutes followed by 98° C. for 2 minutes. One μl of extract is used as template in a PCR reaction to amplify the edited region of genomic DNA using PrimeSTAR® GXL DNA Polymerase (Clontech R050A). The 50 μl reaction contains 5 μl of 5× buffer, 4 μl dNTP, 1 μl template, 2 μl of polymerase, and 3 μl of each 5 μM forward primer (CD46-F1, SEQ ID NO:159) and reverse primer (CD46-R1, SEQ ID NO:160). Cycling conditions are 2 minutes at 94° C., 30 cycles of 10 seconds at 98° C., 15 seconds at 60° C., 30 seconds at 68° C. PCR product can be purified using DNA Clean & Concentrator kit (Zymo D4004). The final product can be sequenced by Illumina MiSeq and analyzed for insertions and deletions (INDELS) indicative of genome editing at the CD46 locus.

### Example 11

### RNAseq Analysis of crRNA Processing in Mammalian Cells

Processing of crRNA was demonstrated by RNAseq analysis of K562 cells expressing an Mmc3 effector-GFP translational fusion gene and a two-repeat multiplexed crRNA array as described in Example 10.

For this assay, 200,000 K562 cells were transfected by nucelofection with 2 μg of the plasmid containing the Mmc3 human codon-optimized gene expressed from the CMV promoter (SEQ ID NO:145) and a two-repeat multiplexed crRNA array described in Example 10. Approximately 800,000 cells were harvested two days after transfection. Small RNA (sRNA) was prepared using the mirVana miRNA Isolation kit (ThermoFisher, cat# AM1560) using methods described by the manufacturer. Enriched sRNA was prepared as a cDNA library using the CATS Small RNASeq kit (Diagenode, cat #C05010044). Based on the protocol from the manufacturer, approximately 7 ng of RNA was used for library construction with 15 amplification cycles. Libraries were sequenced on an Illumina MiSeq sequencing machine.

For analysis, single-end 75 nt-long reads were pre-processed using cutadapt (Martin (2-011) EMBnet.journal Vol. 17, No.1, pp. 10-12; DOI:10.14806/ej.17.1.200). 5′ ends were hard-trimmed by 3 nucleotides (nt), while 3′ ends were adaptively trimmed upstream a poly(A) sequence. This step also removes the Illumina sequencing adapter, since it is placed downstream the poly(A) sequence in Diagenode CATS libraries. Reads shorter than 17 nt after trimming were discarded. Reads were mapped to the human genome primary assembly hg38, supplemented with the crRNA sequence. Alignment was performed using STAR (Dobin et al 2013), with ENCODE parameters for small RNA-seq (protect-us.mimecast.com/s/lTncCJ6EVwU2LKyCVg9MC7?domain=encodeproject.org) Alignments were filtered to retain reads aligned to the crRNA scaffold using samtools. Mappings were visualized using IGV (Broad Institute) or Geneious.

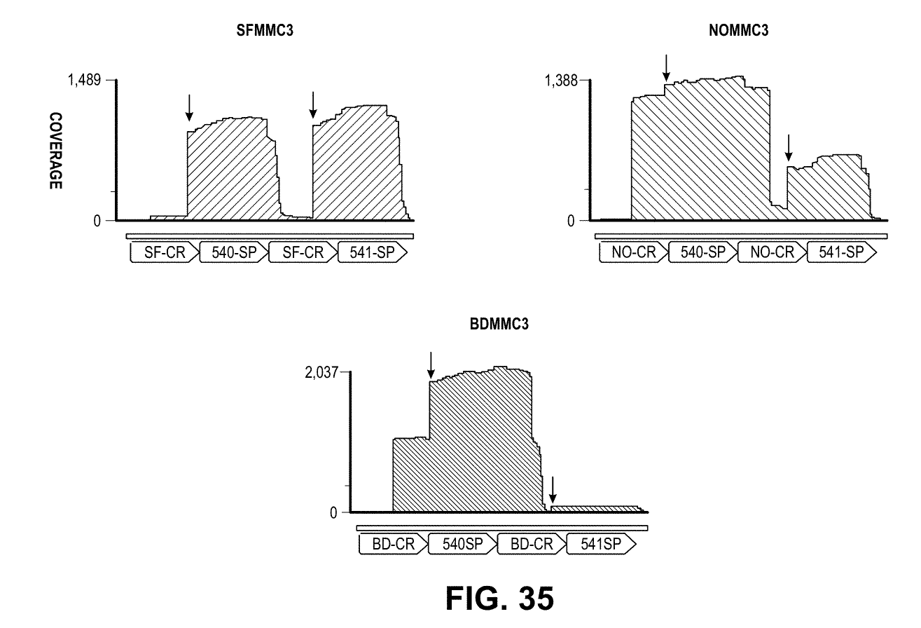

Based on RNAseq performed in bacteria, processed forms were predicted to each include an 18-19 nt sequence derived from the 3′ end of the CRISPR repeat, followed by 20-25 nt derived from the 5′ end of the spacer. Sequences conforming to these specifications were observed for NoMmc3 and SfMmc3 (FIG. 35), confirming that Mmc3 effectors are able to process their own crRNAs and can do this in the context of a mammalian cell.

### Example 12

### Chromosomal Editing in Mammalian HEK293T Cells

The following protocol was used to transfect HEK293T cells with plasmids expressing an Mmc3 effector and a multiplexed crRNA array targeting CD46 as disclosed in Example 10. Lenti-X™ 293 T Cells (Clontech 632180) were grown in DMEM, high glucose, GlutaMAX Supplement, pyruvate (LifeTech 10569044) plus 10% FBS (Clontech 631367) and passaged every other day and split approximately 1:8 so that they were nearly confluent at the time of splitting. (Cells were kept in culture for less than a month before a new vial was thawed.) Cells were dissociated with TrypLE Express Enzyme 1× (Lifetech 12604013) and passaged as a single cell suspension. A nearly confluent plate was split approximately 1:8 as a single cell suspension so that two days later the plate was nearly confluent again. These cells were then plated as a single cell suspension in 24-well plates in 0.6 ml of DMEM/10% FBS per well with 150,000 cells per well one day before transfection. Cells were never allowed to reach 100% confluency before transfection. Constructs for the expression of effectors and guide RNAs (crRNAs) transformed into HEK293T included those of Table 24.

TABLE 25
Effector Constructs used to Transform HEK293T Cells

| | Effector gene (codon optimized | crRNA expression |
|---|---|---|
| Construct | for expression in human cells) | cassette |
| AsCpf1 | SEQ ID NO: 180 | SEQ ID NO: 190 |
| Smp2Cpf1 | SEQ ID NO: 142 | SEQ ID NO: 154 |
| BdMmc3 | SEQ ID NO: 143 | SEQ ID NO: 155 |
| NoMmc3 | SEQ ID NO: 140 | SEQ ID NO: 154 |
| SfMmc3 | SEQ ID NO: 145 | SEQ ID NO: 157 |
| CrpMmc3 | SEQ ID NO: 181 | SEQ ID NO: 191 |
| NapMmc3 | SEQ ID NO: 182 | SEQ ID NO: 192 |
| ObpMmc3 | SEQ ID NO: 183 | SEQ ID NO: 193 |
| SfpMmc3 | SEQ ID NO: 184 | SEQ ID NO: 194 |
| ShMmc3 | SEQ ID NO: 146 | SEQ ID NO: 195 |
| SmpMmc3 | SEQ ID NO: 185 | SEQ ID NO: 196 |
| SvMmc3 | SEQ ID NO: 186 | SEQ ID NO: 158 |
| No2Mmc3 | SEQ ID NO: 187 | SEQ ID NO: 197 |
| PcMmc3 | SEQ ID NO: 188 | SEQ ID NO: 198 |
| Sv2Mmc3 | SEQ ID NO: 189 | SEQ ID NO: 199 |

Cells were transfected one day after plating using Lipofectamine™ LTX Reagent with PLUS™ Reagent (Lifetech 15338100). DNA (0.5 μg) was mixed with 25 μl Serum Free Opti-MEM (Lifetech 51985091) and 0.5 μl of Plus reagent is added. 2 μl of Lipofectamine LTX was diluted in 25 μl of serum free Opti-MEM and added to the diluted DNA. This was incubated at room temperature for 5 minutes before adding to cells. The following day the cells were passaged into one well of a 6 well plate in RPMI 1640 Medium, GlutaMAX Supplement (LifeTech 61870127) plus 10% FBS (Clontech 631367). Two days post transfection the media was changed back to DMEM/10% FBS.

Analysis of Mammalian HEK293T Cells Transformed with Mmc3 Effectors and Guide RNAs

Three days after transfection the cells are made into a single cell suspension, spun down and resuspended in 50 μl FACs buffer and 2 μl CD46 Monoclonal Antibody (MEM-258), APC (Lifetech A15711). Cells are stained 20-30 min in PBS+2% FBS+0.2% sodium azide at 4° C., washed with 750 μl buffer and resuspended in 200-400 μl buffer for flow cytometry analysis performed as provided in Example 10.

Loss of CD46 expression on the cell surface of GFP-expressing cells can be visualized in scatter plots as loss of APC fluorescence with respect to controls (see FIG. 34). To determine the presence of INDELS at both the CD46_540 protospacer and the CD46_541 protospacer the CD46 region can be sequenced as provided in Example 10.

### Example 13

### Mmc3 Editing in Planta

To test for editing ability of Mmc3 in plants, genes encoding the BdMmc3 (SEQ ID NO:161), NoMmc3 (SEQ ID NO:162), NapMmc3 (SEQ ID NO:163), SfMmc3 (SEQ ID NO:164), SmpMmc3 (SEQ ID NO:165), Smp2Mmc3 (SEQ ID NO:166), and FnCpf1 (SEQ ID NO:167) effectors were codon optimized for Oryza sativa (rice) with an N-terminal NLS (SEQ ID NO:168). The engineered Mmc3 genes were cloned into agrobacterial binary vector pCAMBIA1380 under the control of either a ZmUbi promoter (SEQ ID NO:169) or a CaMV promoter (SEQ ID NO:170) and followed by a Nos terminator (SEQ ID NO:171). The pCAMBIA1380 vector includes a gene conferring resistance to hygromycin (HygR). The same vectors included a crRNA expression cassette that included a Rice U6 promoter (SEQ ID NO:172) operably linked to a processed crRNA repeat sequence specific to the Mmc3 being tested, followed by the spacer sequence CAO1sp1 (SEQ ID NO:173), targeting the chlorophyll a oxidase 1 (CAO1) gene, and followed by the U6 terminator (SEQ ID NO:174). In other constructs, the pCAMBIA1380 vector included a CRISPR array targeting two locations in the CAO1 gene that consisted of a rice U6 promoter operably linked to a crRNA repeat specific for the Mmc3 being tested followed by spacer sequence CAO1sp1 (SEQ ID NO:173), followed by another copy of the crRNA repeat sequence, which was followed by a spacer, CAO1sp3 (SEQ ID NO:175), targeting another site in the CAO1 gene, followed by the U6 terminator SEQ ID NO:174).

Agrobacteria-mediated transformation of rice callus was performed essentially as described by Sah et al. (Amadeep Kaur, S. K. S. (2014) Genetic Transformation of Rice: Problems, Progress, and Prospects. Rice Research: Open Access 03(01):1-10.) Briefly, callus tissue was generated by incubating rice grains on callus induction media in light at 28° C. for two weeks which promotes the scutellum to divide and produce callus. Calli were removed from the rice grain and incubated a further two weeks on callus induction medium. For transformation, the callus was combined with Agrobacteria carrying either of the constructs described above. After 20 minutes of gentle shaking the liquid was removed and the callus was air dried for one hour and then placed on co-cultivation medium in the dark at 25° C. for 3 days. The callus was then washed 5 times with an anti-Agrobacterial antibiotic to kill the Agrobacteria. The callus was then placed on selection medium which contained anti-Agrobacterial antibiotic and a low concentration of hygromycin for selection of T-DNA insertions. The plates were incubated in light at 28° C. for six days. The callus was then moved to Selection II medium, which contained a higher concentration of hygromycin. The plates were incubated in light at 28° C. for fourteen days. Hygromycin-resistant calli were removed and either placed on fresh medium to grow larger, or immediately used for preparation of genomic DNA using a standard CTAB method as described in Lukowitz et al., 2000 (Plant Physiology 123: 795-805). Remaining callus was moved to fresh Selection II medium and allowed to grow at 28° C. for an additional fourteen days before re-screening for transgenic callus. Hygromycin-resistant calli were removed and either placed on fresh media to grow larger, or immediately used for preparation of gDNA using standard methods as described above.

Callus can be screened for INDELS at the targeted locations within the CAO1 genes by PCR and next generation sequencing methods. For callus gDNA, PCR with primers Index-Sp1-F1 (SEQ ID NO:176) and Index-Sp1-R2 (SEQ ID NO:177) are used to generate a 202 bp amplicon that spanned the CAO1 region targeted by spacer CAO1sp1. PCR primers Index-Sp3-F2 (SEQ ID NO:247 and Index-Sp3-R2 (SEQ ID NO:179) are used to generate a 157 bp amplicon that spans the CAO1 region targeted by spacer CAO1Sp3 (SEQ ID NO:175). PCR primers Index-Sp3-F1 (SEQ ID NO:178) and Index-Sp3-R2 (SEQ ID NO:179) are used to generate a 208 bp amplicon that spans the CAO1 region targeted by spacer CAO1Sp3. PCR products for each callus are pooled according to the construct tested and purified to remove primers and high molecular weight DNA. A second PCR round is then performed to append Illumina barcodes and sequencing adapters. Indexed amplicons are sequenced on a MiSeq or NextSeq platform. Sequence reads are parsed by barcodes, then trimmed to remove adapter illumina barcode and adaptor sequences. Trimmed sequences are aligned to reference sequences and queried for the presence of INDELS proximal to the specified spacer target site.

Alternatively, gDNA can be used as a template for PCR amplifying the region targeted for mutation and Surveyor assays can be performed to detect calli with indels in the target region. A PCR product that is positive for indels can be cloned and squenced to confirm the location of the indel.

### Example 14

### Targeted Gene Editing in Nannochloropsis gaditana with BdMmc3

A gene that included sequences encoding BdMmc3, codon optimized for Nannochloropsis (SEQ ID NO:209) and that included sequences encoding an NLS, FLAG epitope tag, and flexible linker (SEQ ID NO:210, amino acid sequence SEQ ID NO:211) at the C terminus was operably linked to the RL24 promoter from Nannochloropsis (SEQ ID NO:212) and, at the 5′ end of the BdMmc3 gene, the Nannochloropsis Terminator 2 (SEQ ID NO:213). The expression cassette was designed to express and localize to the nucleus the BdMmc3 effector protein (SEQ ID NO:1) in Nannochloropsis, a Eustigmatophyte algae.

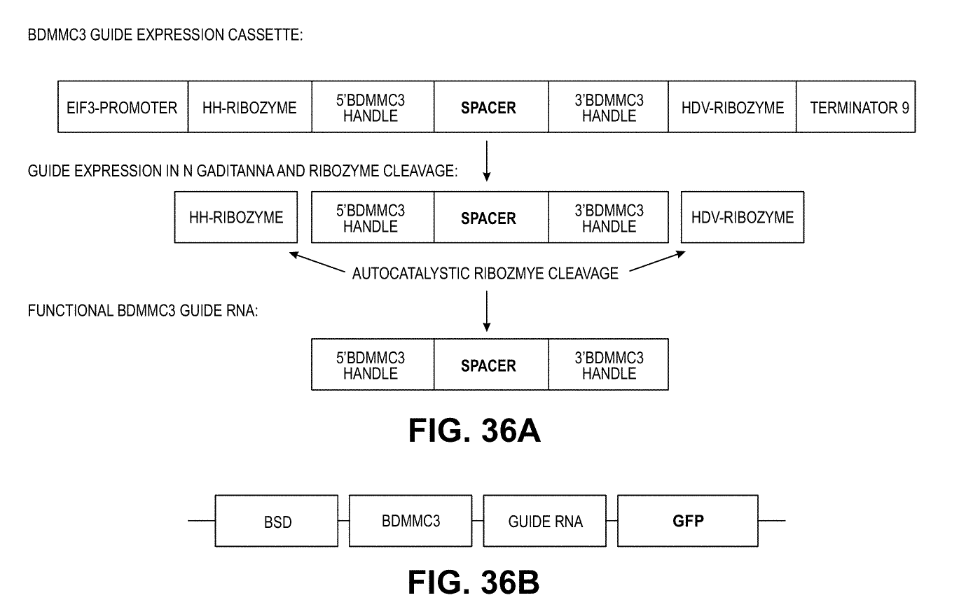

A crRNA expression cassette was also designed for expression in Nannochloropsis. A 28-nucleotide spacer sequence from the LAR1 gene of Nannochloropsis (see, US 2014/0220638, incorporated herein by reference) was cloned so that it was flanked on both the 3′ and 5′ end by the BdMmc3 repeat sequence (SEQ ID NO:28). Three different spacers were tested in the guide constructs, all of which targeted the LAR1 gene: CC1 (SEQ ID NO:214), CC2 (SEQ ID NO:215), and CC3 (SEQ ID NO:216). The repeat-spacer-repeat guide RNA sequence was flanked on the 5′ end by the HH ribozyme sequence (SEQ ID NO:217) and on the 3′ end by the HVD ribozyme sequence (SEQ ID NO:218) (FIG. 36A). The resulting construct was designed for in vivo expression in Nannochloropsis by operably linking a functional N. gaditana promoter (EIF3, SEQ ID NO:219) and terminator (Terminator 9, SEQ ID NO:220) to the 5′ and 3′ ends, respectively of the ribozyme construct. Upon expression in N gaditana, the ribozyme sequence domains will undergo autocatalytic cleavage, which gives rise to a functional processed BdMmc3 guide RNA (FIG. 36A).

The expression cassettes for the BdMmc3 effector and guide RNA described above were cloned into a functional selectable marker cassette for N. gaditana which confers resistance to blasticidin (BSD) and also harbors a green fluorescent protein (GFP) expression cassette as described for expression of Cas9 in US 2017/0073695, incorporated herein by reference. The expression cassettes for BdMmc3 and the guide RNA are cloned in between the BSD and GFP expression cassettes as depicted in FIG. 36B. Expression constructs that included BdMmc3 effector gene and a CC1, CC2, or CC3 guide cassettte were transformed into N. gaditana by electroporation essentially as described in US 2014/0220638, and transformants were selected on agar plates or in liquid medium containing blasticidin.

To examine the LAR1 locus for genome editing events, DNA is isolated from pooled transformants and used to PCR amplify ˜150 to ˜170 bp regions of the genome that encompass the protospacers corresponding to the spacer sequences used in the guides (CC1, CC2, and CC3). PCR amplicons are sequenced and sequences are compared to the wild type locus to determine whether insertions/deletions are present that validate the genome-editing function of BdMmc3.

### Example 15

### Nuclease Assays

Cloning and Preparation of NoORF3

The NoMmc3 ORF3 gene (SEQ ID NO:226 encoding SEQ ID NO:6) was cloned into the pET28 vector using a two step Gibson assembly (Gibson et al. Nature Methods (2009) 6: 343-345). The pET28 vector included a sequence encoding a peptide tag (SEQ ID NO:248) recognized by Streptavidin (IBA Lifesciences, Gottintgen, Germany) that resulted in the peptide tag being added on to the NoMmc3 effector at the C-terminus. The Gibson reaction was used to transform the EPI300 cell line. Four colonies were selected and grown in LB liquid media containing 50 μg/mL Kanamycin for 12 hours at 37° C. Plasmid extractions were performed on each of the cultures. The successful cloning of ORF3 was determined by restriction enzyme analysis and Sanger sequencing.

The sequence-confirmed DNA encoding the tagged ORF3 was used to transform E. coli BL21 (DE3) cells purchased from New England Biolabs (Ipswich, Mass.). The transformants (100 μL) were dispersed on an LB plate containing 50 μg/mL of kanamycin and incubated for 18 hours at 37° C. A single colony was picked to grow in a small 5 mL LB liquid culture containing 50 μg/mL kanamycin. After 8 hours of incubation at 37° C., 1 mL of liquid culture was used to inoculate 1 L of LB media containing 50 μg/mL kanamycin. Cultures were incubated at 37° C. with 200 RPM agitation. Once the cultures reached an O.D. of 0.5, the cultures were placed in a 4° C. cooler for 30 minutes to chill the cultures. Once the cultures were chilled, 0.25 mL of 1M IPTG was added to each flask to induce expression of the ORF3 gene. Cultures were incubated overnight at room temperature.

The next day, cells were harvested by centrifugation at 5000×g for 15 minutes. Cell pellets were solubilized in 25 mM Tris (pH 8.0), 300 mM KCl, 10% glycerol, 5 mM MgCl₂, and 1 mM adenosine-5′-triphosphate (ATP), lysozyme (1 mg/mL), DNaseI (1 U/mL), phenylmethanesulfonyl fluoride (0.1 mg/mL), and complete protease inhibitor tablets were added to the lysis buffer. Cells were re-suspended and lysed by sonication. Cells were pulsed at 70% amplitude with 30 second bursts for a total of 5 minutes. The lysate was frozen in the −80° C. freezer until further use.

The lysate was thawed and centrifuged at 10,500×g for 45 minutes to remove the cell membrane. The supernatant was collected in a 250 mL bottle chilled on ice and loaded onto a 5 mL Streptactin XT superflow column from IBA Lifesciences (Gottintgen, Germany) After the supernatant was run through the column, the column was washed with 2 column volumes of buffer A (25 mM Tris pH 8.0, 300 mM KCl, 10% glycerol, and 5 mM MgCl₂) buffer before starting a linear gradient with buffer B (buffer A containing 5 mM D-desthiobiotin) for 5 column volumes. The protein elution was monitored by UV-vis spectroscopy and fractions containing protein were pooled, analyzed by SDS-PAGE, and concentrated using a molecular weight cutoff of 10 kDa. NoMmc3 ORF3 polypeptide was stored in an eppendorf tube in the −80° C. freezer.

Preparation of Mammalian Cell Lysates

HEK293T mammalian cell lines expressing the AsCpf1 (SEQ ID NO:81) and Smp2Cpf1 (SEQ ID NO:200) effector proteins and crRNAs (SEQ ID NO:190 for the AsCpf1 crRNA and SEQ ID NO:154 for the Smp2Cpf1 crRNA) as provided in Example 10 were cultured on plates prior to harvesing and lysate production. For harvesting cells, plates were placed on ice and the media was removed from the plates and the cells washed in 5 mL of PBS buffer. Lysis buffer (400 μL per plate of a buffer that included 25 mM Tris pH 8.0, 100 mM NaCl, 5% glycerol, 0.2% Triton X100, and 1 protease inhibitor tablet per 10 mL) was added and the cells scraped from the plate. The cell harvesting step was repeated with an additional 300 uL of lysis buffer. The cells were re-suspended in the buffer and centrifuged at 13k×g for 10 minutes at 4° C. The supernatant was aliquoted into chilled Eppendorf tubes (200 μL) and stored in the −80° C. freezer.

Activity Assays with Mammalian Lysate

Assays were conducted in buffer (25 mM Tris pH 8.0, 100 mM NaCl, and 5% glycerol) and contained approximately 500 ng/μL of a pUC19 plasmid that included the CD46 exon 1 (SEQ ID NO:152), 15 μL of lysate, and 10 μM NoMmc3 ORF3 polypeptide. Reactions were performed by incubating in a thermocycler set to 37° C. for 30 minutes and quenched by heat inactivation at 85° C. for 5 minutes. The DNA was extracted using the Genomic DNA Clean & Concentrator kit (Zymogen, Tustin, Calif.). The resulting DNA was eluted with 2×10 μL of nuclease free water. The purified DNA was digested using 0.5 μL PvuI-HF restriction enzyme and 2 μL of 10× CutSmart® buffer (New England Biolabs, Ipswich, Mass.). The restriction digestion was incubated at 37° C. for 1 hour. The resulting digestion was separated on a 1.0% agarose gel and imaged using a Typhoon imager. Cut DNA fragments were quantified using ImageJ software.

In assays containing linearized DNA substrate, The CD46-pUC19 vector was digested with PvuI and linear DNA purified prior to setting up assays. Assays were performed as described above but without the restriction digest step.

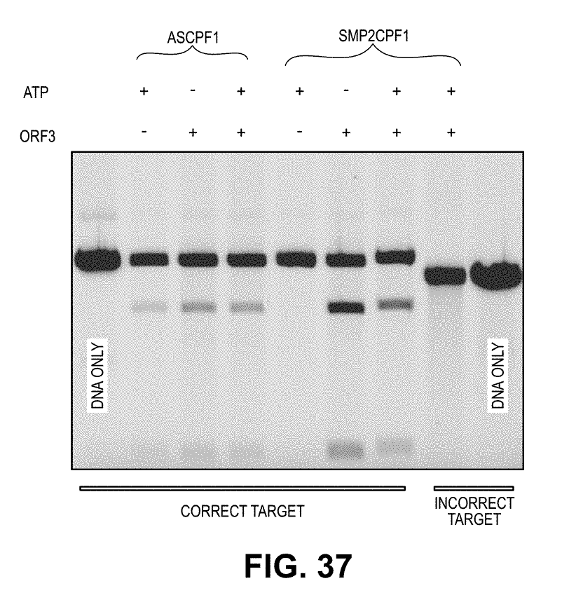

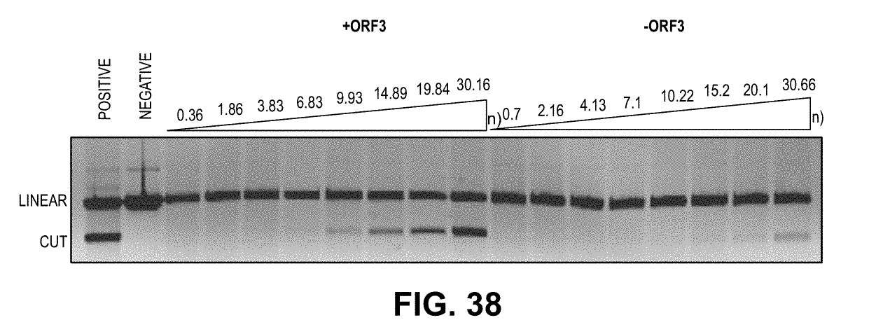

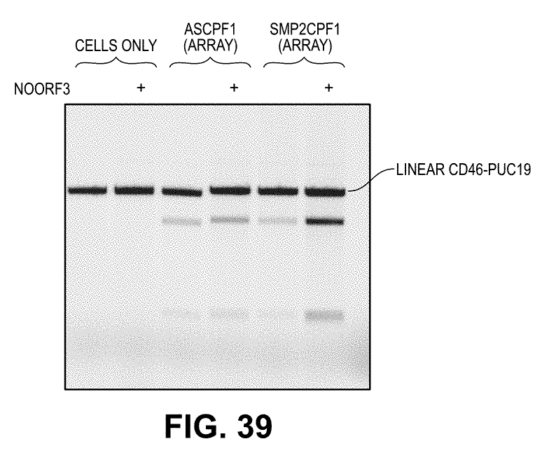

FIG. 37 shows the results of nuclease assays in lysates of HEK293T cells expressing the AsCpf1 and Smp2Cpf1 effector proteins and crRNAs targeting CD46 exon 1 with and without the NoMmc3 ORF3 polypeptide. It can readily be seen that the lower band on the gel which results from cutting of the target plasmid is increased in both Cpf1 effector samples that include Mmc3 ORF3 with respect to the lysates that were assayed without the addition of Mmc3 ORF3. The enhancement of nuclease activity is especially striking for the Smp2Cpf1 effector. FIG. 38 shows a gel with successive samples of a time course of the same assay format using the Smp2Cpf1 effector lysate, in which nuclease activity is observed to increase steadily after about nine minutes in the presence of the Mmc3 ORF3 polypeptide and after about fifteen minutes in the absence of the Mmc3 ORF3 polypeptide, and in which the presence of the Mmc3 ORF3 polypeptide is associated with increased cutting of the target DNA at all timepoints where cutting is observed. FIG. 38 shows a time course of the same assay format using the Smp2Cpf1 effector/crRNA lysate where the crRNA included 22 nt of the NoMmc3 spacer followed by the 540 spacer (SEQ ID:249). The presence of the Mmc3 ORF3 polypeptide results in a consistent increase of nuclease activity after about 9 minutes whereas lack of the Mmc3 ORF3 polypeptide resulted in a slower observable onset of nuclease activity after about 15 minutes. In result, the presence of Mmc3 ORF3 polypeptide is associated with dramatically increased cutting of the target DNA. The results of a 30 minute assay of two Cpf1 effectors, AsCpf1 and Smp2Cpf1, with and without added Mmc3 ORF3 polypeptide are directly compared in FIG. 39. Analysis of band intensities provides that the presence of the ORF3 polypeptide resulted in 1.6 fold the control (no ORF3 polypeptide present) amount of cutting when AsCpf1 was used as the effector, and 9-fold the control level of cutting when Smp2Cpf1 was the effector, for a significantly greater amount of cutting than was observed to occur when AsCpf1 was the effector.

1. An engineered, non-naturally occurring Clustered Regularly Interspersed Short Palindromic Repeat (CRISPR)-CRISPR associated (Cas) (CRISPR-Cas) system comprising:
a) an Mmc3 effector polypeptide, or one or more nucleotide sequences encoding an Mmc3 effector polypeptide, wherein the Mmc3 effector polypeptide:
comprises an amino acid sequence selected from the group comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or
comprises a variant of an Mmc3 effector comprising an amino acid sequence having at least 60%, 65%, 70%, 75%, 80%, 85%, 90%, or 95% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or
comprises the amino acid sequence of a naturally-occurring Mmc3 effector having at least 30% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; and
b) one or more engineered guide RNAs comprising a guide sequence, wherein the one or more guide RNAs is designed to form a complex with the Mmc3 effector polypeptide and wherein the one or more guide RNAs comprises a guide sequence designed to hybridize with one or more target nucleic acid molecules,
wherein the guide RNA and the Mmc3 effector polypeptide do not naturally occur together.

2. A CRISPR-Cas system according to claim 1, wherein the guide RNA forms a complex with the Mmc3 effector and wherein the guide RNA hybridizes to the one or more target nucleic acid molecules, resulting in cleavage of the target nucleic acid molecule.

3-10. (canceled)

11. The CRISPR-Cas system of claim 1, wherein the target nucleic acid is a prokaryotic or a eukaryotic target nucleic acid.

12-15. (canceled)

16. The CRISPR-Cas system of claim 1, wherein the nucleotide sequence encoding the Mmc3 effector polypeptide is codon optimized for expression in a eukaryotic cell.

17. The CRISPR-Cas system of claim 1, wherein the Mmc3 effector polypeptide comprises at least one nuclear localization sequence (NLS).

18. The CRISPR-Cas system of claim 1, comprising two or more guide RNAs.

19-24. (canceled)

25. A method of modifying one or more target nucleic acid sequences in vivo, comprising delivering to a cell comprising one or more nucleic acid molecules comprising one or more target nucleic acid sequences a non-naturally occurring or engineered composition comprising:
a) one or more polynucleotide sequences comprising one or more guide RNAs, or one or more polynucleotide sequences encoding one or more guide RNAs, wherein the one or more guide RNAs is capable of hybridizing with one or more target nucleic acid sequences, and
b) an Mmc3 effector polypeptide, or one or more nucleotide sequences encoding an Mmc3 effector polypeptide; wherein the Mmc3 effector polypeptide:
comprises an amino acid sequence selected from the group comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or
comprises a variant of an Mmc3 effector comprising an amino acid sequence having at least 60%, 65%, 70%, 75%, 80%, 85%, 90%, or 95% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26; or
comprises the amino acid sequence of a naturally-occurring Mmc3 effector having at least 50% identity to an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26;
wherein the one or more guide RNAs form one or more complexes with theMmc3 effector polypeptide, and wherein the one or target nucleic acid molecules is modified by the Mmc3 effector.

26-27. (canceled).

28. The method of claim 25, wherein the percentage of target nucleic acid cleavage is at least 4%, and wherein the target nucleic acid cleavage is determined by plasmid interference assay, PCR, gel electrophoresis, genome sequencing, surveyor assay, and/or a phenotypic assay.

29-33. (canceled)

34. The method of claim 25, wherein the cell is a eukaryotic cell.

35. The method of claim 34, wherein the nucleotide sequence encoding the Mmc3 effector polypeptide is codon optimized for expression in a eukaryotic cell.

36. The method of claim 34, wherein the Mmc3 effector polypeptide comprises one or more NLSs.

37. The method of claim 25, wherein one or more polynucleotide sequences encoding one or more guide RNAs and the nucleotide sequence encoding said Mmc3 effector polypeptide are operably linked to one or more regulatory elements.

38. The method of claim 37, wherein the regulatory element is selected from the group consisting of a promoter, an enhancer, an internal ribosomal entry sites (IRES), a 5′-untranslated region, and a 3′-untranslated region.

39. The method of claim 25, wherein the non-naturally occurring or engineered composition is delivered inside a cell or a cellular organelle via electroporation, nucleofection, lipofection, calcium phosphate precipitation, bacterial conjugation, or a delivery vehicle comprising liposome(s), particle(s), exosome(s), microvesicle(s), a gene-gun, a virus, or one or more viral vector(s).

40. The method of claim 39, wherein the non-naturally occurring or engineered composition is delivered inside a cell or a cellular organelle via a viral vector.

41. The method of claim 25, wherein one or more polynucleotide sequences comprising one or more guide RNAs is delivered to a cell that has previously been transformed with a nucleic acid sequence encoding an Mmc3 effector.

42. The method of claim 25, wherein the non-naturally occurring or engineered composition further comprises:
an Mmc3 ORF3 polypeptide, or one or more nucleotide sequences encoding an Mmc3 ORF3 polypeptide.

43-50. (canceled)

51. An engineered, non-naturally occurring CRISPR-Cas system comprising one or more nucleic acid constructs comprising:
a) a Cpf1 effector polypeptide, or one or more nucleotide sequences encoding a Cpf1 effector polypeptide, wherein the Cpf1 effector polypeptide comprises an amino acid sequence having at least 95% identity to SEQ ID NO:200; and
b) a polynucleotide sequence encoding a guide RNA, wherein the guide RNA is designed to form a complex with the Cpf1 effector polypeptide and wherein the guide RNA comprises a guide sequence designed to hybridize with one or more target nucleic acid molecules,
wherein the guide RNA and the Cpf1 effector polypeptide do not naturally occur together.

52. An engineered, non-naturally occurring CRISPR-Cas system according to claim 51, wherein the polynucleotide sequence encoding the Cpf1 polypeptide and the polynucleotide sequence encoding a guide RNA are located on the same or different nucleic acid constructs of the system, wherein when transcribed, the one or more guide RNAs forms one or more complexes with the Cpf1 effector polypeptide, and wherein the one or more guide RNAs hybridizes to the one or more target nucleic acid molecules, resulting in cleavage of the target nucleic acid molecule.

53. The CRISPR-Cas system of claim 51, wherein the system further comprises an Mmc3 ORF3 polypeptide or a nucleotide sequence encoding an Mmc3 ORF3 polypeptide.
